(19) United States
(12) Patent Application Publication
(10) Pub. No.: US 2024/0132517 A1
(43) Pub. Date: Apr. 25, 2024

(54) MACROCYCLIC COMPOUND, PHARMACEUTICAL COMPOSITION, AND USE THEREOF

(71) Applicant: GOHARMONY THERAPEUTICS (SHENZHEN) CO., LTD., Shenzhen, Guangdong province (CN)

(72) Inventors: Liguang DAI, Beijing (CN); Wei HU, Beijing (CN); Changxin DONG, Beijing (CN); Yanqing YANG, Beijing (CN); Wei WU, Beijing (CN); Wenlian XU, Beijing (CN)

(73) Assignee: GOHARMONY THERAPEUTICS (SHENZHEN) CO., LTD., Shenzhen, Guangdong province (CN)

(21) Appl. No.: 18/276,713

(22) PCT Filed: Feb. 9, 2022
(86) PCT No.: PCT/CN2022/075712

§ 371 Date: Aug. 10, 2023

(30) Foreign Application Priority Data
Feb. 10, 2021 (CN) 202110184793.7

(51) Int. Cl. C07D 498/16; A61K 45/06; A61P 35/00; C07D 498/22; C07D 515/16

(52) U.S. Cl. CPC C07D 498/16; A61K 45/06; A61P 35/00; C07D 498/22; C07D 515/16

(57) ABSTRACT

A compound represented by formula (I), or a pharmaceutically acceptable salt, solvate, active metabolite, polymorph, isotope marker, isomer or prodrug thereof. Also provided are a pharmaceutical composition comprising the same, and the use of the compound and the pharmaceutical composition in the preparation of a medicament for treating tyrosine kinase-mediated diseases. The provided compound and the pharmaceutical composition thereof have significant tyrosine kinase inhibitory activity, can overcome tumor drug resistance, can break through the blood-brain barrier, and further have excellent pharmacokinetic properties and excellent oral bioavailability, and can be administered in a small dose, thereby reducing treatment cost for patients and possible side effects; therefore, the provided compound and the pharmaceutical composition thereof have great application potentials.

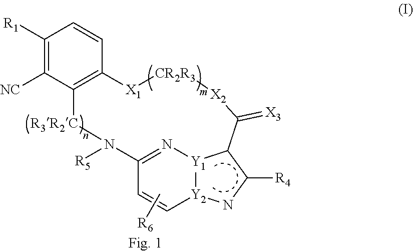

### TECHNICAL FIELD

The present disclosure relates to certain macrocyclic compounds that inhibit SRC, MET and/or CSF1R and other multikinases; pharmaceutical compositions comprising such compounds; and methods for treating cancer by use of such compounds.

### BACKGROUND

Protein kinases are key regulators of cell growth, proliferation and survival. Genetic and epigenetic changes accumulate in cancer cells, resulting in aberrant activation of signal transduction pathways that drive the malignant process (Science. 2002, 298, 1912-1934). Pharmacological inhibition of these signaling pathways presents promising intervention opportunities for targeted cancer therapies (Nature. 2004, 432, 294-297).

The mesenchymal-epithelial transition factor (Met) gene is located on the long arm of human chromosome 7, and contains 21 exons. c-Met is a receptor tyrosine kinase (RTK) with autonomous phosphorylation activity produced by the Met gene code. c-Met and its only known ligand HGF (hepatocyte growth factor) play an important role in cell proliferation, survival, invasion, tissue development and organ regeneration, etc. Abnormal forms of Met gene include mutation, amplification, rearrangement and overexpression. Met rearrangement is very rare in lung cancer. In lung cancer, the probability of c-Met protein overexpression is 33.6%, the probability of c-Met gene amplification is 9.8-20.0%, and the probability of c-Met mutation is 0.8-4.0%. Currently Met or HGF-targeted drugs are mainly divided into two categories: small molecule kinase inhibitors and monoclonal antibodies. Small molecule kinase inhibitors can be further divided into multiple kinase inhibitors (Crizotinib, Cabozantinib, MGCD265, AMG208, Altiratinib and Golvatinib, etc.) and selective Met inhibitors (competitive ATP inhibitors: Capmatinib and Tepotinib [MSC2156119J]; noncompetitive adenosine triphosphate inhibitor: tivantinib). Monoclonal antibodies can be further divided into anti-MET antibodies (onartuzumab and emibetuzumab [LY2875358]) and anti-HGF antibodies (ficlatuzumab [AV-299] and rilotumumab [AMG102]).

SFK is a member of the SRC family, a cytoplasmic tyrosine kinase, and plays an important role in cell signal transduction induced by extracellular stimuli (including growth factors and integrins) (Oncogene, 2004, 23, 7906-7909). Currently, researchers have found and reported increased expression of non-receptor tyrosine kinase SRC and/or increased SRC kinase activity in a variety of human cancers, including breast cancer, colon cancer, lung cancer, and head and neck cancer. Increased expression/activity of SRC and its downstream activation of STAT3 have been reported to be associated with various epithelial cancers, and correlated with the expression of various growth factors such as vascular endothelial growth factor and HGF. SRC is a key downstream transducer of Met-driven tumor growth. SRC activation is essential for both ligand-dependent and non-ligand-independent activation of Met. In Met-driven gastric cancer cell lines, SRC inhibition increases the sensitivity of cells to c-Met inhibition, providing a theoretical basis for the combination therapy of c-Met inhibitors and SRC inhibitors (Clin Cancer Res. 2010, 16, 3933-3943). Although HGF/Met signaling is associated with the occurrence and development of colorectal cancer (CRC), the therapeutic effect of Met inhibitors alone is not obvious. Combined inhibition of Met and SRC enhances cell proliferation and inhibition of apoptosis in mutant and wild-type RAS cells (Exp Ther Med. 2017. 4692).

CSF1R (colony-stimulating factor 1 receptor, CSF1R) is a colony-stimulating factor 1 receptor. Colony-stimulating factor (CSF1) is a cytokine that controls the production, differentiation and function of macrophages. Uncontrolled inflammation in the tumor microenvironment is a hallmark of cancer and is associated with M2-polarized macrophages. A tumor associated macrophage (Tumor associated macrophage, TAM) is more similar to M2 polarized macrophages and play an important role in promoting cancer proliferation, invasion and metastasis (J Hematol Oncol. 2017, 10, 58). The tumor-promoting function of TAM is based on its ability to secrete pro-angiogenic and growth factors and its ability to strongly inhibit T cell effect function by releasing immunosuppressive cytokines and affecting T cell metabolism (Cancer Cell. 2014, 25, 846-859). While anti-PD-1 monoclonal antibodies (mAbs) that target immune checkpoints have shown benefit in treating some cancers, these drugs are not consistently effective. In patients with advanced solid tumors, survival of TAM is mediated by signaling through CSF1R, and inhibition of CSF1R signaling decreases TAM and increases CD8/CD4⁺ T cell ratios. Therefore, CSF1R signaling that targets TAM regulation is a promising therapeutic strategy for solid tumors, either as monotherapy or in combination therapy. Coexpression of CSF1R with CSF1 was most commonly detected in aggressive tumors. Activation of CSF1R promotes Src-dependent disruption of mammary epidermal architecture, suggesting that targeted inhibition of CSF-1R and SRC may be a valuable strategy for the treatment of aggressive tumors. Tenosynovial giant cell tumor (TGCT) or pigmented villonodular synovitis (PVNS) is a clonal neoplastic proliferation produced by CSF1-overexpressing cells that recruit CSF1R-loaded polyclonal macrophages and constitute the tumor bulk. The use of small molecule inhibitors of CSF1R can improve the affected junctions (Curr Opin Oncol. 2011, 23, 361-366).

In summary, multi-kinase inhibition such as MET/SRC/CSF1R has great potential for the treatment of cancer. But so far there are no clinically available compounds that inhibit Met/Src and/or CSF1R. The MET/SRC/CSF1R and other multi-kinase inhibitors of the present disclosure have a good effect in the brain, and will significantly make up for unmet clinical needs.

### SUMMARY

An object of the present disclosure is to provide a novel compound, or a pharmaceutically acceptable salt, solvate, active metabolite, polymorph, isotope marker, isomer or prodrug thereof, which possesses excellent tyrosine kinase inhibitory activity.

Another object of the present disclosure is to provide a pharmaceutical composition.

Another object of the present disclosure is to provide use of the novel compound, or a pharmaceutically acceptable salt, solvate, active metabolite, polymorph, isotope marker, isomer or prodrug thereof.

The present disclosure provides a compound represented by formula (I), or a pharmaceutically acceptable salt, solvate, active metabolite, polymorph, isotope marker, isomer or prodrug thereof,

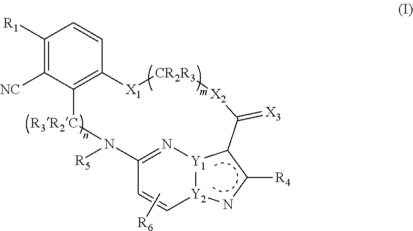

wherein,
X₁ is selected from the group consisting of —O—, —S— and —NR₁₁—;
X₂ is selected from the group consisting of —CH₂—, —O—, —S— and —NH—;
X₃ is selected from the group consisting of O, S and NR₁₀;
Y₁ and Y₂ are different and are selected from the group consisting of C and N;

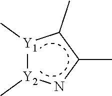

the circular dashed line in indicates that there is a conjugated double bond in the ring;
R₁, R₄, R₅, R₆, R₁₀, and R¹¹ are each independently selected from the following groups which are substituted or unsubstituted: hydrogen, halogen, C_{1˜8} alkyl, deuterated C_{1˜8} alkyl, C_{1˜8} alkoxy, C_{1˜8} alkylamino, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl or cyano;
R₂ and R₃ are each independently selected from the following groups which are substituted or unsubstituted: hydrogen, halogen, C_{1˜8} alkyl, C_{1˜8} alkoxy, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl, cyano; or, together with the C atom and the X₂ group to which they are connected, form a 3˜10 membered cycloalkyl group, a 3˜10 membered heterocyclic group containing at least one heteroatom, or a 5˜10 membered heteroaryl group containing at least one heteroatom;
or, when X₁ is —NR₁₁—, the N atom and the C atom in CR₂R₃ together with R₁₁ and R₂ form a 3˜10 membered azacycloalkyl group;
R_{2′} and R_{3′} are each independently selected from the following groups which are substituted or unsubstituted: hydrogen, halogen, C_{1˜8} alkyl, C_{1˜8} alkoxy, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl, cyano; or, together with the connected C and the adjacent N atom, form a 3˜10 membered heterocyclic group containing at least one heteroatom or a 5˜10 membered heteroaryl group containing at least one heteroatom;
m, n represent an integer from 1 to 10; wherein m represents an integer from 3 to 10 when Y₁=C, Y₂=N, X₃=O, X₂=NH and R₆=H;
the substituents of the aforementioned groups may be selected from the group consisting of halogen, C_{1˜8} alkyl, C_{1˜8} haloalkyl, C_{1˜8} alkoxy, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl and cyano.

Further, in the above formula (I), R₁, R₄, R₅, R₆, R₁₀, R₂, R₃, R_{2′}, R_{3′} and their optional substituents can represent groups including, but not limited to: hydrogen, deuterium, fluorine, chlorine, bromine, methyl, ethyl, propyl, isopropyl, n-butyl, sec-butyl, tert-butyl, methoxy, ethoxy, propoxy, isopropoxy, —CN, —CF₃, —NH₂, —NH(C_{1˜4} alkyl), —N(C_{1˜4} alkyl)₂, —CO₂C_{1˜4} alkyl, —CO₂ H, —NHC(O)C_{1˜4} alkyl, —SO₂ C_{1˜4} alkyl, —C(O)NH₂, —C(O)NH(C_{1˜4} alkyl), —C(O)N(C_{1˜4} alkyl)₂, cyclopropyl, cyclobutyl, cyclopentyl, cyclohexyl, pyrrolidinyl, pyrazolyl, piperidinyl, pyridyl, piperazinyl, triazinyl, furyl, thiofuryl, morpholinyl, thiomorpholinyl, phenyl, naphthyl, biphenyl, terphenyl, etc.

In one embodiment, the compound has a structure of the following formula I-1 or I-2:

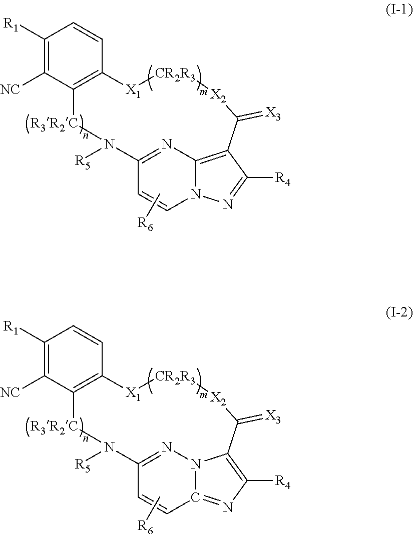

wherein the definition of each group is as described above;
and, when X₂ is NH, X₃=O, and m=1 to 2, R₆ is not H.

In one embodiment, the compound has a structure of the following formula I-11, preferably formula I-12 or I-13;

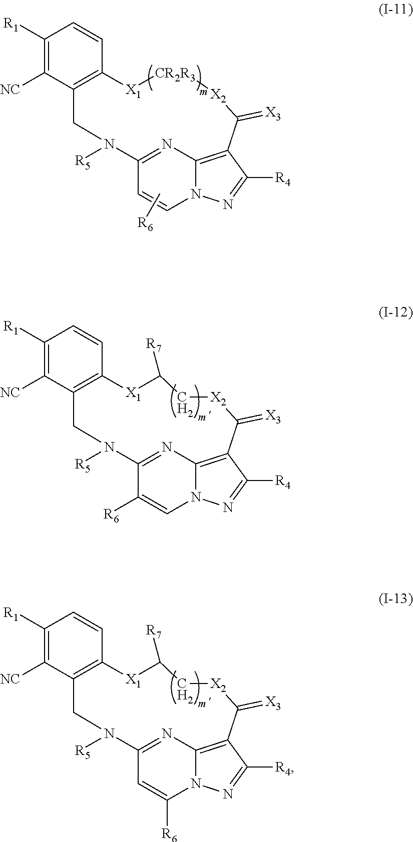

wherein the definition of each group is as described above,
in formulas I-12 and I-13, R₇ is each independently selected from the following groups which are substituted or unsubstituted: hydrogen, halogen, C_{1˜8} alkyl, deuterated C_{1˜8} alkyl, C_{1˜8} alkoxy, C_{1˜8} alkylamino, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl or cyano; m′ represents an integer from 1 to 10.

In one embodiment, the compound has a structure of the following formula I-21, preferably formula I-22 or I-23;

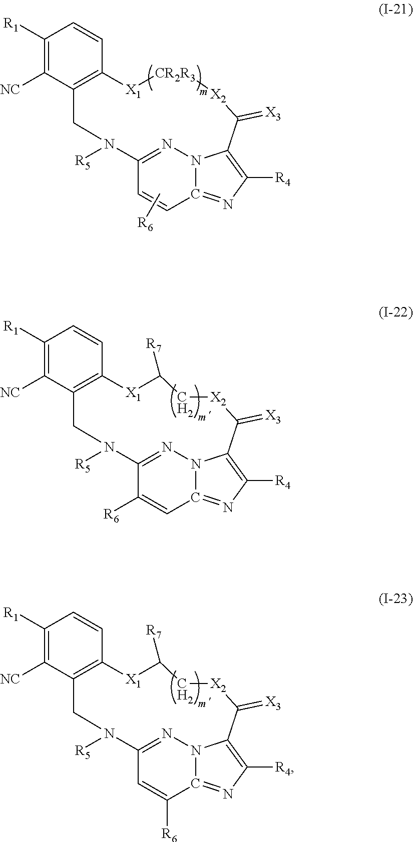

wherein the definition of each group is as described above,
in formulas I-22 and I-23, R₇ is each independently selected from the following groups which are substituted or unsubstituted: hydrogen, halogen, C_{1˜8} alkyl, deuterated C_{1˜8} alkyl, C_{1˜8} alkoxy, C_{1˜8} alkylamino, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl or cyano; m′ represents an integer from 1 to 10.

In another aspect, the present disclosure also provides a compound represented by formula (II), or a pharmaceutically acceptable salt, solvate, active metabolite, polymorph, isotope marker, isomer or prodrug thereof,

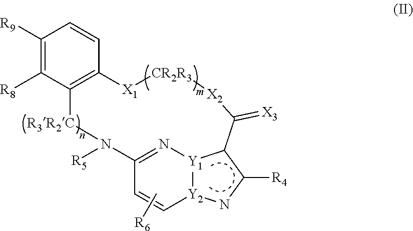

wherein,
X₁ is selected from the group consisting of —O—, —S— and —NR₁₁—;
X₂ is selected from the group consisting of —CH₂—, —O—, —S— and —NH—;
X₃ is selected from the group consisting of O, S and NR₁₀;
Y₁ and Y₂ are different and are selected from the group consisting of C and N;
the circular dashed line in

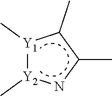

indicates that there is a conjugated double bond in the ring;
R₄, R₅, R₆, R₁₀, and R₁₁ are each independently selected from the following groups which are substituted or unsubstituted: hydrogen, halogen, C_{1˜8} alkyl, deuterated C_{1˜8} alkyl, C_{1˜8} alkoxy, C_{1˜8} alkylamino, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl or cyano;
R₂ and R₃ are each independently selected from the following groups which are substituted or unsubstituted: hydrogen, halogen, C_{1˜8} alkyl, C_{1˜8} alkoxy, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl, cyano; or, together with the C atom and the X₂ group to which they are connected, form a 3˜10 membered cycloalkyl group, a 3˜10 membered heterocyclic group containing at least one heteroatom, or a 5˜10 membered heteroaryl group containing at least one heteroatom;
or, when X₁ is —NR₁₁—, the N atom and the C atom in CR₂R₃ together with R₁₁ and R₂ form a 3˜10 membered azacycloalkyl group;
R_{2′} and R_{3′} are each independently selected from the following groups which are substituted or unsubstituted: hydrogen, halogen, C_{1˜8} alkyl, C_{1˜8} alkoxy, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl, cyano; or, together with the connected C and the adjacent N atom, form a 3˜10 membered heterocyclic group containing at least one heteroatom or a 5˜10 membered heteroaryl group containing at least one heteroatom;
R₈ and R₉ are each independently halogen;
m and n represent an integer from 1 to 10;
the substituents of the aforementioned groups may be selected from halogen, C_{1˜8} alkyl, C_{1˜8} haloalkyl, C_{1˜8} alkoxy, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl or cyano.

The compound has a structure of the following formula II-1 or II-2:

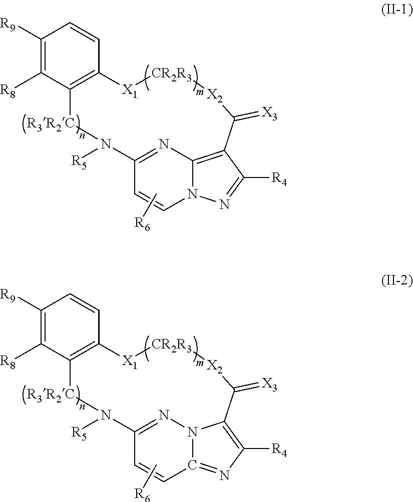

wherein the definition of each group is as described above.

In one embodiment, the compound has a structure of the following formula II-11, preferably formula II-12 or II-13;

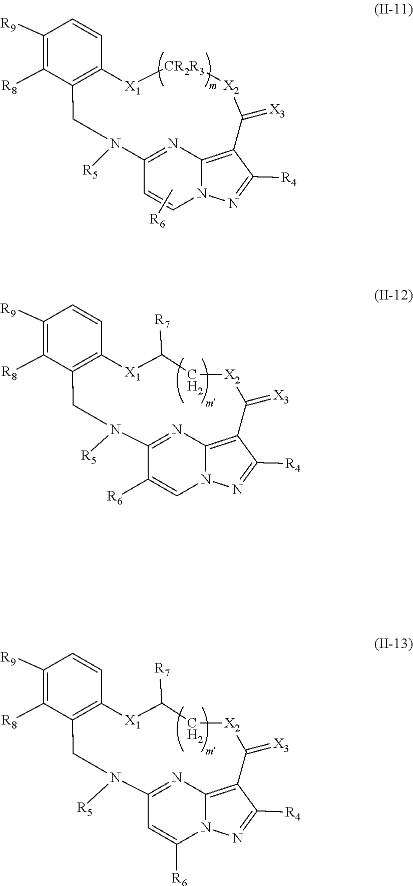

wherein the definition of each group is as described above,
in formulas II-12 and II-13, R₇ is each independently selected from the following groups which are substituted or unsubstituted: hydrogen, halogen, C_{1˜8} alkyl, deuterated C_{1˜8} alkyl, C_{1˜8} alkoxy, C_{1˜8} alkylamino, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl or cyano; m′ represents an integer from 1 to 10.

In one embodiment, the compound has a structure of the following formula II-21, preferably formula II-22 or II-23;

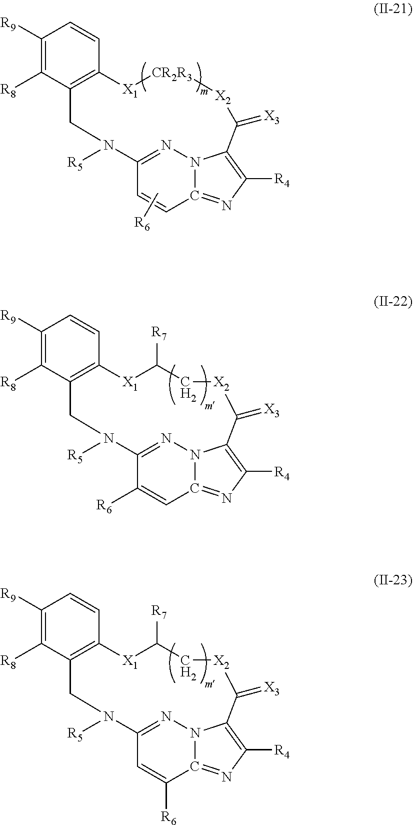

wherein the definition of each group is as described above,
in formulas II-22 and II-23, R₇ is each independently selected from the following substituted or unsubstituted groups: hydrogen, halogen, C_{1˜8} alkyl, deuterated C_{1˜8} alkyl, C_{1˜8} alkoxy, C_{1˜8} alkylamino, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl or cyano; m′ represents an integer from 1 to 10.

In one embodiment, R₁ is F; or, R₉ is F.

In one embodiment, R₅ is selected from C_{1˜8} alkyl, C_{1˜8} haloalkyl, deuterated C_{1˜8} alkyl, C_{3˜8} cycloalkyl C_{1˜8} alkyl or cyano C_{1˜8} alkyl; preferably ethyl, deuteroethyl, cyclopropylmethyl or cyanomethyl.

In one embodiment, R₄ is hydrogen or amino.

In one embodiment, R₆ is selected from hydroxyl, amino, C_{1˜8} alkoxy or C_{1˜8} alkylamino; preferably, R₆ is hydroxyl; or, R₆ is amino; or, R₆ is C_{1˜8} alkoxy; or, R₆ is C_{1˜8} alkylamino.

In one embodiment, X₁ is —O—.

In one embodiment, X₂ is —O—; or, X₂ is —NH—.

In one embodiment, X₃ is —O—; or, X₃ is NR₁₀, and R₁₀ is selected from hydroxyl or C_{1˜8} alkoxy.

In one embodiment, m, n and m′ may represent an integer of 1, 2, 3, 4, 5 or 6, especially 1, 2 or 3.

In one embodiment, in the above formulas, when the substituents R₂ and R₃ on the C atom are each independently selected from forming cycloalkyl, heterocyclyl or heteroaryl together with the X₂ group, it means that R₂ or R₃ of one —(CR₂R₃)— group adjacent to the X₂ group forms together with its connected C and the X₂ group.

In one embodiment according to the present disclosure, in the above formulas, when R₂ and R₃ are each independently a single bond connecting the C atom and the adjacent macrocyclic ring atom, the adjacent macrocyclic ring atom may be a ring atom C in another adjacent —(CR₂R₃)— group, or may also be a ring atom in an adjacent X₂ group. In a preferred embodiment, R₂ and R₃ in the —(CR₂R₃)— group adjacent to the X₂ group may each independently be a single bond connecting the C atom and the X₂ group, i.e., R₂ and/or R₃ is a single bond connecting the C atom and the central atom of X₂. For example, when one of R₂ and R₃ is a single bond, the X₂ group forms one double bond with the adjacent —(CR₂R₃)— group; when two of R₂ and R₃ are single bonds, the X₂ group forms one triple bond with the adjacent —(CR₂R₃)— group.

Similarly, between two adjacent —(CR₂R₃)— groups in the macrocycle, a substituted or unsubstituted double bond or triple bond may also be formed between C and C atoms.

When X₁ is selected from —NR₁₁—, the N atom and the C atom in CR₂R₃ together with R₁₁ and R₂ may form a 3˜10 membered azacycloalkyl group, for example, azetidinyl.

In one embodiment according to the present disclosure, in the above formulas, the substituents R₂ and R₃ on each C atom are independently selected from the following groups which are substituted or unsubstituted: hydrogen, halogen, C_{1˜5} alkyl, C_{1˜5} alkoxy, C_{1˜5} haloalkyl, C_{3˜6} cycloalkyl, or R₂, R₃ are each independently a single group connecting the C atom and the adjacent macrocyclic ring atom.

In one embodiment according to the present disclosure, in the above formulas, R_{2′} and R_{3′} are each independently selected from the following groups which are substituted or unsubstituted: hydrogen, halogen, C_{1˜5} alkyl, C_{1˜5} alkoxy, C_{1˜5} haloalkyl, C_{3˜6} cycloalkyl; or, together with the connected C and L₂, form a 4˜8 membered heterocyclic group containing at least one heteroatom.

In one embodiment according to the present disclosure, in the above formulas, the macrocyclic atom C in the —CR₂R₃—group or the —CR_{2′}R_{3′}—group or the C in the substituents can produce one or more chiral centers depending on the different groups, and the present disclosure includes all optical isomers and racemates.

In one embodiment according to the present disclosure, in the above formulas, R₄ is selected from the following groups which are substituted or unsubstituted: hydrogen, halogen, C_{1˜5} alkyl, C_{1˜5} alkoxy, C_{1˜5} haloalkyl, C_{3˜6} cycloalkyl, hydroxyl, mercapto, carboxyl, amino or cyano.

In one embodiment according to the present disclosure, the optional substituents in the above embodiments are selected from the group consisting of fluorine, bromine, —CN, —OH, —CF₃, —NH₂, —NHH(C_{1˜4} alkyl), —NH(C_{1˜4} alkyl)₂, —CO₂C_{1˜4} alkyl, —CO₂H, —NHHC(O)C_{1˜4} alkyl, —SO₂C_{1˜4} alkyl, —C(O)NH₂, —C(O)NH(C_{1˜4} alkyl), —C(O)N(C_{1˜4} alkyl)₂, C_{1˜5} alkyl, C_{3˜6} cycloalkyl, C_{3˜6} heterocyclyl, C_{6˜10} aryl and C_{5˜10} heteroaryl.

In one embodiment according to the present disclosure, the compound is selected from the following compounds:

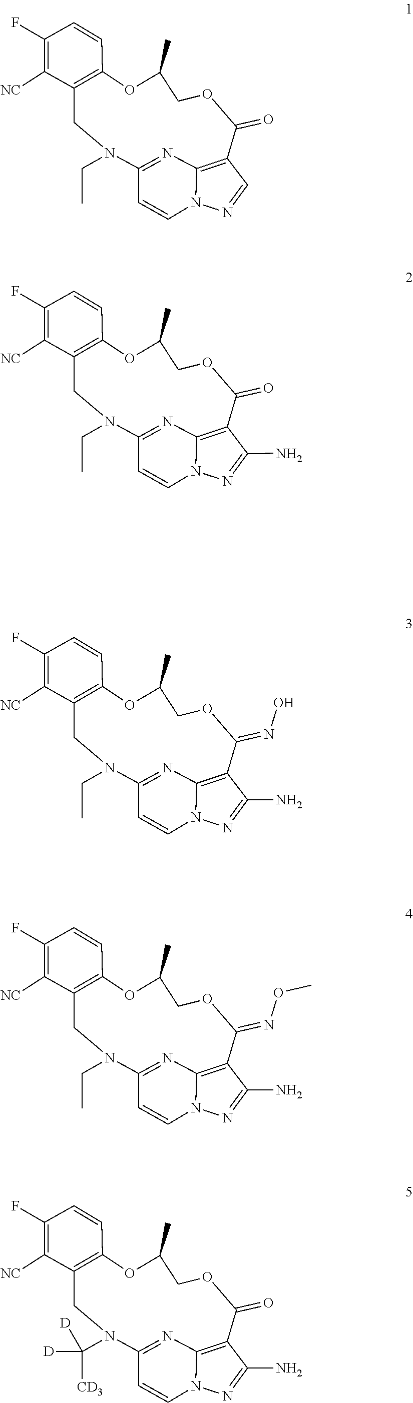 [SMILES: CCN1Cc2c(ccc(F)c2C#N)O[C@@H](C)COC(=O)c2cnn3ccc1nc23 ; CCN1Cc2c(ccc(F)c2C#N)O[C@@H](C)COC(=O)c2c(N)nn3ccc1nc23 ; CCN1Cc2c(ccc(F)c2C#N)O[C@@H](C)CO/C(=N\O)c2c(N)nn3ccc1nc23 ; CCN1Cc2c(ccc(F)c2C#N)O[C@@H](C)CO/C(=N\OC)c2c(N)nn3ccc1nc23 ; [2H]C([2H])([2H])C([2H])([2H])N1Cc2c(ccc(F)c2C#N)O[C@@H](C)COC(=O)c2c(N)nn3ccc1nc23]

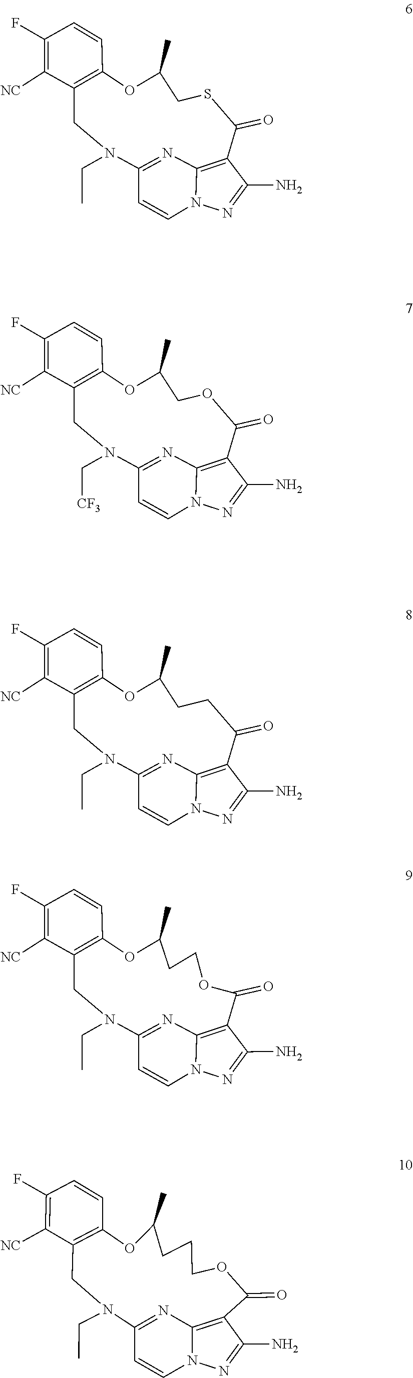 [SMILES: CCN1Cc2c(ccc(F)c2C#N)O[C@@H](C)CSC(=O)c2c(N)nn3ccc1nc23 ; C[C@H]1COC(=O)c2c(N)nn3ccc(nc23)N(CC(F)(F)F)Cc2c(ccc(F)c2C#N)O1 ; CCN1Cc2c(ccc(F)c2C#N)O[C@@H](C)CCC(=O)c2c(N)nn3ccc1nc23 ; CCN1Cc2c(ccc(F)c2C#N)O[C@@H](C)CCOC(=O)c2c(N)nn3ccc1nc23 ; CCN1Cc2c(ccc(F)c2C#N)O[C@@H](C)CCCOC(=O)c2c(N)nn3ccc1nc23]

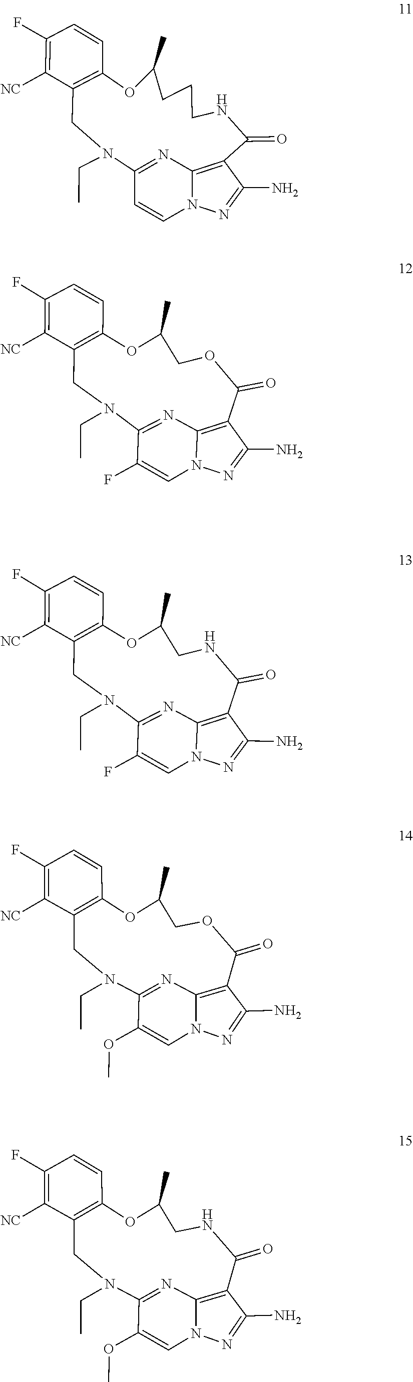 [SMILES: CCN1Cc2c(ccc(F)c2C#N)O[C@@H](C)CCCNC(=O)c2c(N)nn3ccc1nc23 ; CCN1Cc2c(ccc(F)c2C#N)O[C@@H](C)COC(=O)c2c(N)nn3cc(F)c1nc23 ; CCN1Cc2c(ccc(F)c2C#N)O[C@@H](C)CNC(=O)c2c(N)nn3cc(F)c1nc23 ; CCN1Cc2c(ccc(F)c2C#N)O[C@@H](C)COC(=O)c2c(N)nn3cc(OC)c1nc23 ; CCN1Cc2c(ccc(F)c2C#N)O[C@@H](C)CNC(=O)c2c(N)nn3cc(OC)c1nc23]

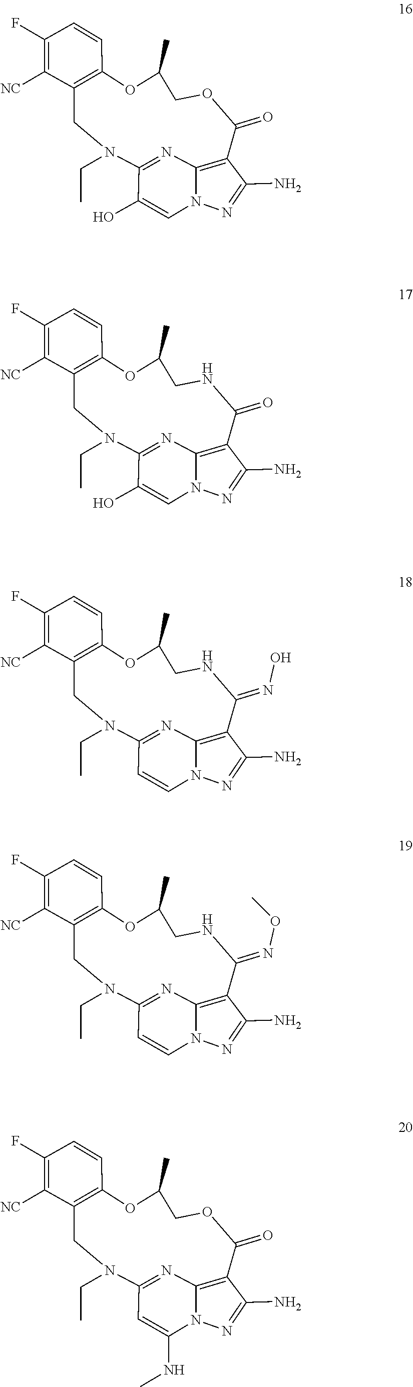 [SMILES: CCN1Cc2c(ccc(F)c2C#N)O[C@@H](C)COC(=O)c2c(N)nn3cc(O)c1nc23 ; CCN1Cc2c(ccc(F)c2C#N)O[C@@H](C)CNC(=O)c2c(N)nn3cc(O)c1nc23 ; CCN1Cc2c(ccc(F)c2C#N)O[C@@H](C)CN/C(=N\O)c2c(N)nn3ccc1nc23 ; CCN1Cc2c(ccc(F)c2C#N)O[C@@H](C)CN/C(=N\OC)c2c(N)nn3ccc1nc23 ; CCN1Cc2c(ccc(F)c2C#N)O[C@@H](C)COC(=O)c2c(N)nn3c(NC)cc1nc23]

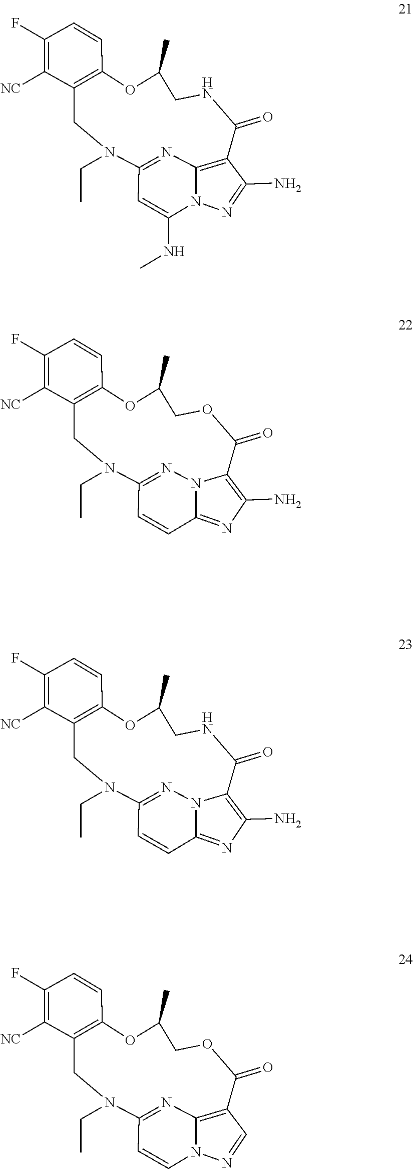 [SMILES: CCN1Cc2c(ccc(F)c2C#N)O[C@@H](C)CNC(=O)c2c(N)nn3c(NC)cc1nc23 ; CCN1Cc2c(ccc(F)c2C#N)O[C@@H](C)COC(=O)c2c(N)nc3ccc1nn23 ; CCN1Cc2c(ccc(F)c2C#N)O[C@@H](C)CNC(=O)c2c(N)nc3ccc1nn23 ; CCN1Cc2c(ccc(F)c2C#N)O[C@@H](C)COC(=O)c2cnn3ccc1nc23]

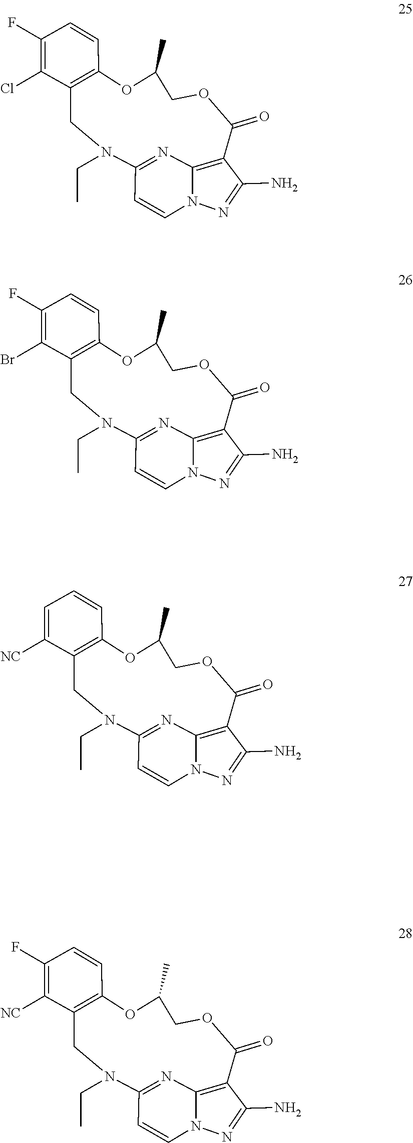 [SMILES: CCN1Cc2c(ccc(F)c2Cl)O[C@@H](C)COC(=O)c2c(N)nn3ccc1nc23 ; CCN1Cc2c(ccc(F)c2Br)O[C@@H](C)COC(=O)c2c(N)nn3ccc1nc23 ; CCN1Cc2c(C#N)cccc2O[C@@H](C)COC(=O)c2c(N)nn3ccc1nc23 ; CCN1Cc2c(ccc(F)c2C#N)O[C@H](C)COC(=O)c2c(N)nn3ccc1nc23]

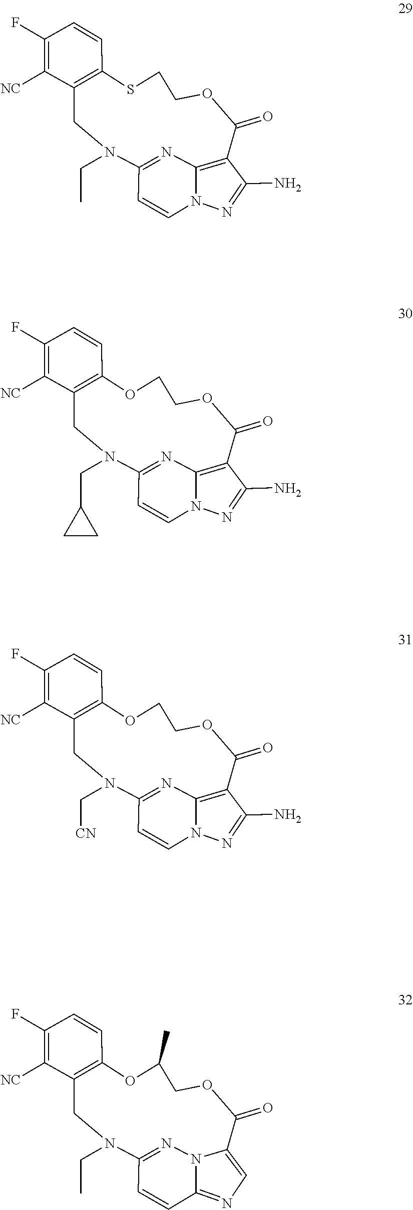 [SMILES: CCN1Cc2c(ccc(F)c2C#N)SCCOC(=O)c2c(N)nn3ccc1nc23 ; N#Cc1c(F)ccc2c1CN(CC1CC1)c1ccn3nc(N)c(c3n1)C(=O)OCCO2 ; N#CCN1Cc2c(ccc(F)c2C#N)OCCOC(=O)c2c(N)nn3ccc1nc23 ; CCN1Cc2c(ccc(F)c2C#N)O[C@@H](C)COC(=O)c2cnc3ccc1nn23]

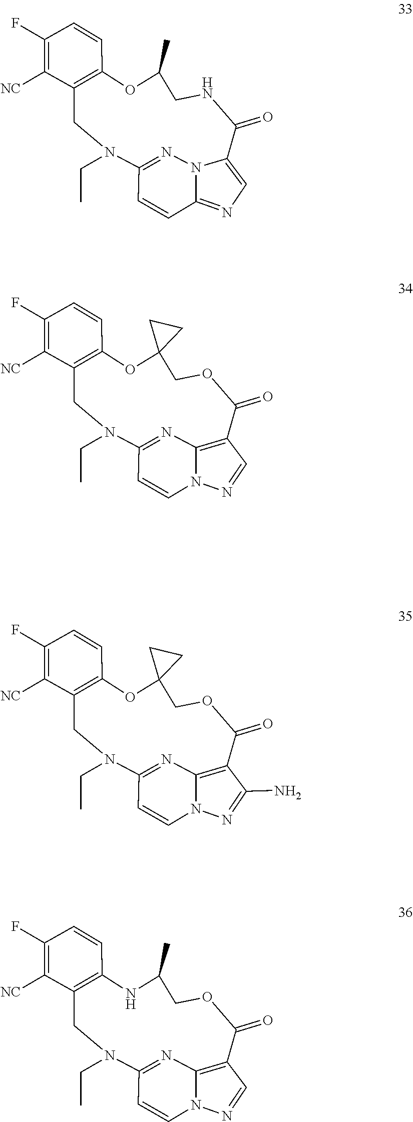 [SMILES: CCN1Cc2c(ccc(F)c2C#N)O[C@@H](C)CNC(=O)c2cnc3ccc1nn23 ; CCN1Cc2c(ccc(F)c2C#N)OC2(CC2)COC(=O)c2cnn3ccc1nc23 ; CCN1Cc2c(ccc(F)c2C#N)OC2(CC2)COC(=O)c2c(N)nn3ccc1nc23 ; CCN1Cc2c(ccc(F)c2C#N)N[C@@H](C)COC(=O)c2cnn3ccc1nc23]

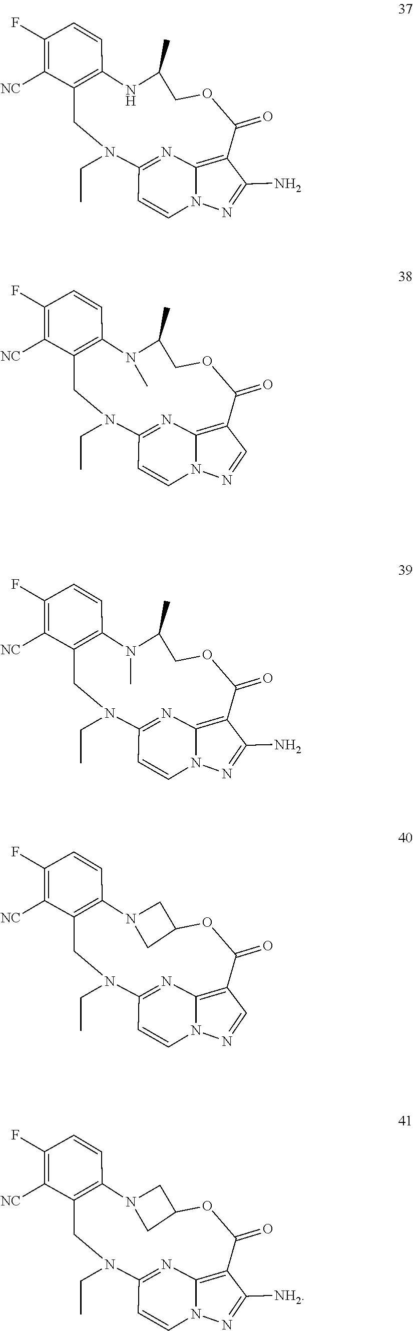 [SMILES: CCN1Cc2c(ccc(F)c2C#N)N[C@@H](C)COC(=O)c2c(N)nn3ccc1nc23 ; CCN1Cc2c(ccc(F)c2C#N)N(C)[C@@H](C)COC(=O)c2cnn3ccc1nc23 ; CCN1Cc2c(ccc(F)c2C#N)N(C)[C@@H](C)COC(=O)c2c(N)nn3ccc1nc23 ; CCN1Cc2c(ccc(F)c2C#N)N2CC(C2)OC(=O)c2cnn3ccc1nc23 ; CCN1Cc2c(ccc(F)c2C#N)N2CC(C2)OC(=O)c2c(N)nn3ccc1nc23]

The present disclosure also provides a pharmaceutical composition comprising the compound, or a pharmaceutically acceptable salt, solvate, active metabolite, polymorph, isotope marker, isomer or prodrug thereof as described in any one of the above technical solutions, and a pharmaceutically acceptable carrier.

The pharmaceutical composition includes, but not limited to, oral dosage forms, parenteral dosage forms, external dosage forms, rectal dosage forms, and the like. For example, the pharmaceutical composition may be tablets, capsules, pills, powders, sustained release formulations, solutions and suspensions for oral administration; sterile solutions, suspensions or emulsions for parenteral injection; ointments, cream, gel, and the like, for external administration; or suppositories for rectal administration.

The pharmaceutical composition may also include other active ingredients or drugs in comination with the compound, or a pharmaceutically acceptable salt, solvate, active metabolite, polymorph, isotope marker, isomer or prodrug thereof.

The present disclosure also provides use of the above-mentioned compound, or a pharmaceutically acceptable salt, solvate, active metabolite, polymorph, isotope marker, isomer or prodrug thereof, and use of the above-mentioned pharmaceutical composition, in the preparation of a medicament for tyrosine kinase-mediated diseases; and a method for treating tyrosine kinase-mediated diseases, which comprises administering to a patient an effective amount of the above-mentioned compound, or a pharmaceutically acceptable salt, solvate, active metabolite, polymorph, isotope marker, isomer or prodrug thereof, or the above-mentioned pharmaceutical composition.

Further, the tyrosine kinase is selected from one or more of the following: SRC, MET, CSF1R, ALK, ROS1, TRKA, TRKB, TRKC, JAK2, SRC, FYN, LYN, YES, FGR, FAK, AXL, ARK5.

Further, the tyrosine kinase-mediated diseases include cancer, pain, neurological disorders, autoimmune diseases and inflammation.

Still further, the tyrosine kinase-mediated cancer may include lung cancer, colorectal cancer, breast cancer, ovarian cancer, thyroid cancer, prostate cancer, hepatocellular carcinoma, renal cell carcinoma, gastric and esophageal cancer, cholangiocarcinoma, glioma, glioblastoma, head and neck cancer, inflammatory myofibroblastic tumor, angiosarcoma, epithelioid hemangioendothelioma, anaplastic large cell lymphoma, and the like.

Still further, the tyrosine kinase-mediated pain man be pain of any origin or etiology, including cancer pain, chemotherapy pain, neuropathic pain, injury pain or other origins.

Still further, the tyrosine kinase-mediated autoimmune diseases include rheumatoid arthritis, Sjogren syndrome, type I diabetes, lupus, and the like.

Still further, the tyrosine kinase-mediated neurological disorders include Alzheimer's Disease, Parkinson's Disease, Amyotrophic Lateral Sclerosis, Huntington's disease, and the like.

Still further, the tyrosine kinase-mediated inflammatory diseases include atherosclerosis, allergy, inflammation caused by infection or injury, and the like.

The present disclosure also provides a combination for treating cancer in a patient, which comprises a therapeutically effective amount of a formulation for inhibiting SRC and MET and/or CSF1R and an additional anticancer agent in the same or different specifications, administered simultaneously or separately, wherein the formulation for inhibiting SRC and MET and/or CSF1R comprises the compound or a pharmaceutically acceptable salt, solvate, active metabolite, polymorph, isotope marker, isomer or prodrug thereof according to any one of claims 1-15, or the pharmaceutical composition according to claim 16; the additional anticancer agent is an EGFR antibody or an EGFR small molecule inhibitor, preferably selected from cetuximab, nexituzumab, panitumumab or Amivantamab double antibody, afatinib, brigatinib, canertinib, dacomitinib, erlotinib, gefitinib, HKI 357, lapatinib, Osimertinib, Nakotinib, Nazartinib, Neratinib, Omotinib, Pelitinib, PF-06747775, Roxitinib, Vandetanib, Amitinib,

Vometinib, Mobocertinib, DZD9008, BEBT-109, lazertinib, CLN-081, WTS-004, JFAN-1001, C-005, XZP-5809-TT1, JRF103, FWD1509, JNJ-372, or a pharmaceutically acceptable salt thereof.

The diaryl macrocyclic compound and the pharmaceutical composition thereof provided by the present disclosure have significant tyrosine kinase inhibitory activity, can overcome tumor drug resistance, can break through the blood-brain barrier, have excellent pharmacokinetic properties and excellent oral bioavailability, and can be administered in a small dose, thereby reducing treatment costs for patients and possible toxic and side effects; therefore, they have great application potentials. Moreover, the inventors of the present disclosure found that when there is a cyano group (—CN) at the ortho position on the benzene ring of the macrocyclic compound and the amino hydrogen in the ring is replaced by an alkyl group or other groups, it can have a significant effect on the activity of the compound, for example, increase the kinase inhibitory activity for SRC, MET, CSF1R and improve the selectivity for other kinases. The inventors of the present disclosure have also found that when there are two halogen groups at the ortho and meta positions on the benzene ring of the macrocyclic compound, especially when the meta position is F, it can also have a significant impact on the activity of the compound, for example, increase the kinase inhibitory activity for SRC, MET, CSF1R, etc. The inventors of the present disclosure found that when some groups of the macrocyclic compound are comprehensively changed, the permeability of the compound is significantly improved, and the blood-brain barrier permeability is increased.

### BRIEF DESCRIPTION OF THE DRAWINGS

FIG. 1 shows a histogram of average concentrations in rat brain tissue and blood plasma after gavage administration of the compound of Example 1; and

FIG. 2 shows a histogram of average concentrations in rat brain tissue and plasma after gavage administration of TPX-0022.

### DETAILED DESCRIPTION

### Definitions

In this application, each group may have the following definitions:

Hydrogen may be represented as —H, and can also be replaced with isotopes such as deuterium and tritium.

Halogen may include fluorine, chlorine, bromine, iodine.

C_{1˜8} alkyl may include methyl, ethyl, n-propyl, isopropyl, 2-methyl-1-propyl, 2-methyl-2-propyl, 2-methyl-1-butyl, 3-methyl-l-butyl, 2-methyl-3-butyl, 2,2-dimethyl-1-propyl, 2-methyl-1-pentyl, 3-methyl-1-pentyl, 4-methyl-l-pentyl, 2-methyl-2-pentyl, 3-methyl-2-pentyl, 4-methyl-2-pentyl, 2,2-dimethyl-I-butyl, 3,3-dimethyl-1-butyl, 2-ethyl-1-butyl, n-butyl, isobutyl, sec-butyl, tert-butyl, n-pentyl, isopentyl, neopentyl, tert-amyl, hexyl, heptyl, octyl, etc.

Deuterated C_{1˜8} alkyl and tritiated C_{1˜8} alkyl may indicate that one or more, or even all, hydrogen atoms on C_{1˜8} alkyl are replaced with isotopes such as deuterium and tritium.

C_{1˜8} alkoxy may be represented as —OC_{1˜8} alkyl, wherein the C_{1˜8} alkyl includes groups as defined above. For example, C_{1˜8} alkoxy may include methoxy, ethoxy, n-propoxy, isopropoxy, n-butoxy, isobutoxy, sec-butoxy, tert-butoxy, etc.

C_{1˜8} haloalkyl may be represented as a group obtained after any number of hydrogen atom(s) in C_{1˜8} alkyl is(are) replaced with halogen, wherein the C_{1˜8} alkyl and halogen include groups as defined above. For example, C_{1˜8} haloalkyl may include —CF₃, etc.

C_{3˜8} cycloalkyl may be represented as a non-aromatic saturated carbocycle, including monocarbocycle (having one ring) and bicarbocycle (having two rings). For example, C_{3˜8} cycloalkyl may include

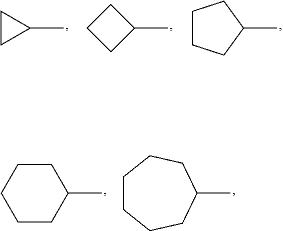

etc.

C_{3˜8} cycloalkyl C_{1˜8} alkyl may be represented as C_{1˜8} alkyl with C_{3˜8} cycloalkyl, wherein the definitions of C_{3˜8} cycloalkyl and C_{1˜8} alkyl are as mentioned above. For example, C_{3˜8} cycloalkyl C_{1˜8} alkyl may include cyclopropylmethyl, cyclobutylmethyl, cyclohexylethyl, etc.

C_{3˜8} heterocyclyl may be represented as a group obtained after any number of ring atom(s) in C_{3˜8} cycloalkyl is(are) replaced with a heteroatom such as O, S, N, P, Si, etc., wherein the C_{3˜8} cycloalkyl includes groups as defined above. For example, C_{3˜8} heterocyclyl may include oxiranyl, thioethyl, azetidinyl, azetidinyl, oxetanyl, thiabutanyl, tetrahydrofuranyl, pyrrolidinyl, oxazolidinyl, tetrahydropyrazolyl, pyrrolinyl, dihydrofuranyl, dihydrothiophenyl, piperidinyl, tetrahydropyranyl, tetrahydrothiopyranyl, morpholinyl, piperazinyl, dihydropyridyl, tetrahydropyridyl, dihydropyranyl, tetrahydropyranyl, dihydrothiopyranyl, azepanyl, oxepanyl, thiepanyl, oxazabicyclo[2.2.1 ]heptyl, azaspiro[3.3]heptyl, etc.

C_{6˜20} aryl may include monocyclic aryl, bicyclic aryl, or aryl with more rings. For example, C_{6˜20} aryl may include phenyl, biphenyl, naphthyl, phenanthrenyl, anthracenyl, azulenyl, etc.

C_{5˜20} heteroaryl may represent an unsaturated group containing any number of heteroatom(s) such as O, S, N, P, Si as ring atoms. For example, C_{5˜20} heteroaryl may include pyrrolyl, furyl, thienyl, imidazolyl, oxazolyl, pyrazolyl, pyridyl, pyrimidinyl, pyrazinyl, quinolinyl, isoquinolyl, tetrazolyl, triazolyl, triazinyl, benzofuryl, benzothienyl, indolyl, isoindolyl, etc.

Hydroxyl may be represented as —OH.

Mercapto may be represented as —SH.

Carboxyl may be represented as —COOH.

An ester group may be represented as —COOR′, and the definition of R′ may be the definition of the substituents described in formula (1). For example, an ester group substituted by C_{1˜8} alkyl may be represented as —COOC_{1˜8} alkyl, wherein the C_{1˜8} alkyl includes groups as defined above.

Acyl may be represented as —COR′, and the definition of R′ can be the definition of the substituents described in formula (1). For example, acyl substituted by C_{1˜8} alkyl may be represented as —COC_{1˜8} alkyl, wherein the C_{1˜8} alkyl inlcudes groups as defined above.

Amino may be represented as —NH₂, —NHHR′ or —NH(R′)₂, and the definition of R′ can be the definition of the substituents described in formula (1). For example, amino substituted by C_{1˜8} alkyl may be represented as —NHHC_{1˜8} alkyl or —NH(C_{1˜8} alkyl)₂, wherein the C_{1˜8} alkyl includes groups as defined above.

Amido may be represented as —CO-amino, wherein the amino is as defined above.

Sulfonyl can be represented as —S(O)₂R′, wherein the definition of R′ may be the definition of the substituents described in formula (1). For example, sulfonyl substituted by C_{1˜8} alkyl can be represented as —S(O)₂C_{1˜8} alkyl, wherein the C_{1˜8} alkyl includes groups as defined above.

Cyano can be represented as —CN.

In the aforementioned definitions, when the number of carbon atoms changes, the above definitions only vary depending on the change of the number of carbon atoms, and does not affect the definition of the group type. For example, “C_{1˜5} alkyl” may include all the groups that meets the number of carbon atoms being 1 to 5 in the definition of “C_{1˜8} alkyl” as mentioned above, such as methyl, ethyl, n-propyl, isopropyl, n-butyl, isobutyl, sec-butyl, tert-butyl, n-amyl, isopentyl, neopentyl, etc.

### DETAILED DESCRIPTION

Unless defined otherwise, all technical and scientific terms used herein have the same meaning as commonly understood by those skilled in the art to which the claimed subject matter belongs. All patents, patent applications, and publications cited herein are hereby incorporated by reference in their entirety unless otherwise indicated. When a trade name appears herein, it is intended to refer to its corresponding product or its active ingredient.

It is to be understood that both the foregoing summary and the following detailed description are exemplary and explanatory only and are not restrictive of the inventive subject matter herein. In this application, it must be noted that, unless the context clearly indictates otherwise, the singular forms used in the specification and claims include the plural forms of the objects referred to. It should also be noted that the use of “or” means “and/or” unless stated otherwise. Furthermore, use of the terms “comprise”, “include” as well as other forms thereof, such as “comprising”, “including”, is not limiting.

Definitions of Standard Chemical Terms can be found in literature works, including Carey and Sundberg, “Advanced Organic Chemistry 4^{th} Ed, Vol A (2000) and B (2001), Plenum Press, New York. Unless otherwise stated, conventional methods within the skill of the art, such as mass spectrometry, NMR, HPLC, protein chemistry, biochemistry, recombinant DNA techniques, and pharmacological methods are adopted. Unless specific definitions are proposed, relevant chemical nomenclature and laboratory procedures and techniques herein in terms of analytical chemistry, organic synthetic chemistry, and medical and medicinal chemistry are known to those skilled in the art. Standard techniques can be used for chemical synthesis, chemical analysis, drug preparation, formulation, drug delivery and treatment of patients. Standard techniques can be used for recombination DNA, oligonucleotide synthesis, and tissue culture and transformation (e.g., electroporation, lipid infection). For example, a kit with instructions provided by the manufacturer can be used, or reactions and purification technique can be carried out according to methods well known in the art, or according to the method described in the present disclosure. Generally speaking, aforementioned technique and steps can be implemented by conventional methods well known in the art and described in various general literatures or more specific literatures, and these literatures are cited and discussed in this invention.

When a substituent is described by a conventional chemical formula written from left to right, the substituent also includes chemically equivalent substituents obtained when the structural formula is written from right to left. For example, CH₂O is equivalent to OCH₂.

The term “substituted” means that any one or more hydrogen atoms on a specific atom are replaced with a substituent, as long as the valence of the specific atom is normal and the substituted compound is stable. When the substituent is oxo (i.e., ═O), it means that two hydrogen atoms are replaced, and oxo does not occur on the aromatic group.

When any variable (such as R) occurs more than once in the composition or structure of a compound, its definition at each occurrence is independent. Thus, for example, if a group is substituted with 0 to 2 Rs, said group may optionally be substituted with up to two Rs, with independent options for each occurrence of R. Also, combinations of substituents and/or variations thereof are permissible only if such combinations result in stable compounds.

C_{m˜n} as used herein means that there are m-n carbon atoms in the moiety. For example, the “_{1˜8} ” group means that the moiety has 1-8 carbon atoms, that is, the group contains 1 carbon atom, 2 carbon atoms, 3 carbon atoms, . . . , 8 carbon atoms atom. Therefore, for example, “C_{1˜8} alkyl” refers to an alkyl containing 1-8 carbon atoms, that is, the alkyl is selected from the group consisting of methyl, ethyl, propyl, isopropyl, n-butyl, isobutyl, sec-butyl, tert-butyl, . . . , octyl, etc. Numerical ranges herein, such as “1-8” refer to each integer in the given range, such as “1-8 carbon atoms” means that the group may have 1 carbon atom, 2 carbon atoms, 3 carbon atoms, 4 carbon atoms, 5 carbon atoms, 6 carbon atoms, 7 carbon atoms or 8 carbon atoms.

The term “member” refers to the number of skeletal atoms that make up the ring. For example, pyridine is a six-membered ring and pyrrole is a five-membered ring.

The term “pharmaceutically acceptable” refers to those compounds, materials, compositions and/or dosage forms which, within the scope of reliable medical judgment, are suitable for use in contact with human and animal tissues without excessive toxicity, irritation, allergic reaction or other problems or complications, commensurate with a reasonable benefit/risk ratio.

The term “pharmaceutical composition” refers to a biologically active compound optionally in admixture with at least one pharmaceutically acceptable chemical ingredient or agent, known as a “carrier,” which facilitates the introduction of compounds into cells or tissues, including but not limited to stabilizers, diluents, suspending agents, thickeners and/or excipients.

The term “pharmaceutically acceptable salt” refers to a salt that retains the biological effectiveness of a free acid and a free base of the specified compound and has no biological or other adverse effects. Unless otherwise specified, as salts in the present disclosure, metal salts, ammonium salts, salts with organic bases, salts with inorganic acids, salts with organic acids, salts with basic or acidic amino acids, and the like, may be mentioned. Non-limiting examples of metal salts include, but are not limited to, alkali metal salts, such as sodium salts, potassium salts, and the like; alkaline earth metal salts, such as calcium salts, magnesium salts, barium salts, and the like; aluminum salts, and the like. Non-limiting examples of salts with organic bases include, but are not limited to, salts with trimethylamine, triethylamine, pyridine, picoline, 2,6-lutidine, ethanolamine, diethanolamine, triethanolamine, cyclohexylamine, dicyclohexylamine, and the like. Non-limiting examples of salts with inorganic acids include, but are not limited to, salts with hydrochloric acid, hydrobromic acid, nitric acid, sulfuric acid, phosphoric acid, and the like. Non-limiting examples of salts with organic acids include, but are not limited to, salts with formic acid, acetic acid, trifluoroacetic acid, fumaric acid, oxalic acid, malic acid, maleic acid, tartaric acid, citric acid, succinic acid, methanesulfonic acid, benzenesulfonic acid, p-toluenesulfonic acid, and the like. Non-limiting examples of salts with basic amino acids include, but are not limited to, salts with arginine, lysine, ornithine, and the like. Non-limiting examples of salts with acidic amino acids include, but are not limited to, salts with aspartic acid, glutamic acid, and the like.

Pharmaceutically acceptable salts can be synthesized from a parent compound containing an acid group or a base group by conventional chemical methods. In general, such salts are prepared by reacting the free acid or base form of these compounds with a stoichiometric amount of the appropriate base or acid in water or an organic solvent or a mixture of both. Generally, non-aqueous media such as ether, ethyl acetate, ethanol, isopropanol or acetonitrile are preferred.

The term “solvate” refers to a physical aggregation of a compound of the present disclosure with one or more solvent molecules, the physical aggregation including varying degrees of ionic and covalent bonds, such as hydrogen bonds. It has been shown that this solvate can be isolated, for example, when one or more solvent molecules are mixed in the crystal lattice. “Solvate” includes two parts, solvent phase and isolatable solvate. There are many examples of corresponding solvates, including ethanol solvates, methanol solvates, and the like. “Hydrate” is a solvate with water (H₂O) molecules as the solvent. One or more compounds of the present disclosure may optionally be prepared as solvates. The preparation of solvates is well known. For example, M. Caira et al, J. Pharmaceutical Sci., 93(3), 601-611 (2004) describes the preparation of a solvate of the antifungal drug fluconazole, i.e., using ethyl acetate and water. E. C. van Tonder et al, AAPS PharmSciTech., 5(1), article 12 (2004); and A. L. Bingham et al, Chem. Commun., 603-604 (2001) also describe similar preparations of solvates and hydrates method. A typical, non-limiting preparation process is to dissolve the compound of the present disclosure in the desired amount of ideal solvent (organic solvent or water or their mixed solvent) at a temperature higher than normal temperature, cool down, and place for crystallization. The crystals are then isolated and picked out by standard methods. The presence of the solvent (water) that forms the solvate (hydrate) in the crystals can be confirmed by IR spectroscopy.

The term “active metabolite” refers to an active derivative of a compound that is formed when the compound is metabolized.

The term “polymorph” refers to a compound of the present disclosure that exist in different crystal lattice forms.

The term “isotopically labeled” refers to an isotopically labeled compound of the present disclosure. For example, the isotopes in the compounds of the present disclosure may include various isotopes of elements such as H, C, N, O, P, F, S, for example, ²H, ³H, ¹³C, ¹⁴C, ¹⁵N, ¹⁸O, ¹⁷O, ³¹P, ³²P, ³⁵S, ¹⁸F and ³⁶S.

The term “pharmaceutically acceptable prodrug” or “prodrug” refers to any pharmaceutically acceptable salt, ester, salt of an ester, or other derivatives of a compound of the present disclosure, which, after administered to a subject, is capable of directly or indirectly providing compounds of the present disclosure or pharmaceutically active metabolites or residues thereof. Particularly preferred derivatives or prodrugs are those compounds which, when administered to a patient, increase the bioavailability of the compounds of the present disclosure (e.g., allow the orally administered compound to be more readily absorbed into the bloodstream), or those compounds which promote the delivery of the parent compound to biological organs or action sites such as the brain or lymphatic system. Prodrugs can be prepared by modifying functional groups present in a compound, either by routine manipulation, or in vivo, in such a way that they decompose to the parent compound. Various prodrug forms are well known in the art. Discussions about prodrugs are provided in, see, T. Higuchi and V. Stella, Pro-drugs as Novel Delivery Systems (1987) Vol. 14 of the ACS Symposium Series, Bioreversible Carriers in Drug Design, (1987) Edward B. Roche, ed., American Pharmaceutical Association, Pergamon Press, Design of Prodrugs, Bundgaard, A. Ed., Elseview, 1985 and Method in Enzymology, Widder, K. et al., Ed .; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, pp. 113-191; and Bundgaard, H., Advanced Drug Delivery Review, 1992, 8, 1-38, the above documents are incorporated herein by reference.

The term “stereoisomer” refers to an isomer resulting from a different spatial arrangement of atoms in a molecule. The compounds of the present disclosure contain structures such as asymmetric or chiral centers, double bonds, etc. Therefore, the compounds of the present disclosure may include various isomers such as optical isomers, geometric isomers, tautomers, atropisomers, and the like. These isomers and their single isomers, racemates, etc., are included in the scope of the present disclosure. For example, for optical isomers, optically active (R)- and (S)-isomers as well as D- and L-isomers can be prepared by chiral resolution, chiral synthesis or chiral reagents, or other conventional techniques. For example, diastereoisomers can be converted by reaction with an appropriate optically active substance (such as a chiral alcohol or Mosher's acid chloride), separated and converted (e.g., hydrolyzed) to the corresponding single isomers. As another example, separation can also be performed by a chromatographic column.

The “pharmaceutical composition” herein can be prepared in a manner well known in the art of pharmacy, and they can be given or administered by a variety of routes, depending on whether local or systemic treatment is desired and the area to be treated. Topical (e.g., transdermal, dermal, ocular, and mucous membranes, including intranasal, vaginal, and rectal delivery), pulmonary (e.g., by inhalation or insufflation of powder or aerosol, including by nebulizer; intratracheal, intranasal), oral or parenteral administration. Parenteral administration includes intravenous, intraarterial, subcutaneous, intraperitoneal or intramuscular injection or infusion; or intracranial, e. g., intrathecal or intracerebroventricular, administration. It can be administered parenterally in the form of a bolus, or it can be administered, for example, by means of a continuous infusion pump. The pharmaceutical composition herein includes, but are not limited to, the following forms: tablets, pills, powders, lozenges, sachets, cachets, elixirs, suspensions, emulsions, solutions, syrups, aerosols (solid or sovled in liquid vehicles); ointments, soft and hard gelatin capsules, suppositories, sterile injectable solutions and sterile packaged powders, and the like, containing, for example, up to 10% by weight of the active compound.

The pharmaceutical composition herein may be formulated in unit dosage form, each dose may contain about 0.1-1000 mg, usually about 5˜1000 mg, more usually about 100-500 mg of the active ingredient. The term “unit dosage form” refers to physically discrete units suitable as unitary dosages for human patients and other mammals, each unit containing a predetermined quantity of active material calculated to produce the desired therapeutic effect, in association with a suitable pharmaceutical carrier.

The term “individual” refers to an individual suffering from a disease, disorder or condition, etc., including mammals and non-mammals. Examples of mammals include, but are not limited to, any member of the Mammalia: humans, non-human primates (such as chimpanzees and other apes and monkeys); livestock such as cattle, horses, sheep, goats, pigs; domesticated Animals, such as rabbits, dogs, and cats; laboratory animals, including rodents, such as rats, mice, and guinea pigs.

The term “treatment” and other similar synonyms include alleviating, mitigating or ameliorating the symptoms of a disease or condition, preventing other symptoms, ameliorating or preventing the underlying metabolic cause of the symptoms, inhibiting the disease or condition, e.g. arresting the progression of the disease or condition, ameliorating the disease or condition, improving a disease or condition, alleviating symptoms caused by a disease or condition, or halting symptoms of a disease or condition. In addition, the term may also include the purpose of prophylaxis. The term also includes obtaining a therapeutic and/or prophylactic effect. The therapeutic effect refers to curing or ameliorating the underlying disease being treated. Also, a cure or amelioration of one or more physical symptoms associated with the underlying disease is a therapeutic effect, e. g., an improvement in a patient's condition is observed although the patient may still be affected by the underlying disease. As to a prophylactic effect, the composition or compound may be administered to a patient at risk for a particular disease, or to a patient presenting with one or more physiological symptoms of the disease even if the disease has not yet been diagnosed.

The term “amount necessary to obtain a therapeutic effect” or “therapeutically effective amount” refers to an amount of at least one agent or compound which, upon administration, is sufficient to alleviate to some extent one or more symptoms of the disease or condition being treated. The result may be a reduction and/or alleviation of a sign, symptom or cause, or any other desired change in a biological system. Effective amounts suitable for any individual case can be determined using techniques such as dose escalation assays. The actual amount of compound, pharmaceutical composition or agent administered will generally be determined by a physician based on relevant circumstances, including the condition being treated, the route of administration chosen, the actual compound being administered; the age, weight and response of the individual patient; the patient's symptoms severity, etc.

The ratio or concentration of the compound of the present disclosure in the pharmaceutical compositions may vary, depending on various factors including dosage, chemical properties (eg, hydrophobicity), route of administration, and the like. For example, a compound of the present disclosure may be provided for parenteral administration in an aqueous physiologically buffered solution containing about 0.1-10% w/v of the compound. Some typical dosage ranges are about 1 μg/kg to about 1 g/kg body weight/day. In certain embodiments, the dosage ranges from about 0.01 mg/kg to about 100 mg/kg body weight/day. The dosage will likely depend on such variables as the type and extent of the disease or condition, the general health of the particular patient, the relative biological potency of the compound selected, the formulation of the excipient and its route of administration.

The term “administration” refers to a method capable of delivering a compound or composition to the desired site of biological action. These methods include, but are not limited to, oral routes, transduodenal routes, parenteral injection (including intravenous, subcutaneous, intraperitoneal, intramuscular, intraarterial injection or infusion), topical and rectal administration. Administration techniques useful for the compounds and methods described herein are well known to those skilled in the art, for example, as discussed in Goodman and Gilman, The Pharmacological Basis of Therapeutics, currented .; Pergamon; and Remington's, Pharmaceutical Sciences (current edition), Mack Publishing Co., Easton, Pa.

The term “IC₅₀” refers to 50% inhibition of a maximal effect obtained in an assay measuring such an effect.

In order to make the purpose, technical solutions and advantages of the present disclosure clearer, the technical solutions of the exemplary embodiments of the present disclosure will be further described below.

The present disclosure can prepare the compounds described in the present disclosure by the following methods. The following methods and examples are intended to illustrate these methods. These procedures and examples should not be construed as limiting the present disclosure in any way. The compounds described herein can also be synthesized using standard synthetic techniques known to those skilled in the art, or using a combination of methods known in the art and methods described herein.

The chemical reactions of the Examples of the present disclosure are completed in a suitable solvent, and the solvent must be suitable for the chemical changes of the present disclosure and the reagents and materials required therefor. In order to obtain the compounds of the present disclosure, it is sometimes necessary for those skilled in the art to modify or select synthetic steps or reaction schemes on the basis of existing embodiments.

An important consideration in planning any synthetic route in this field is the selection of suitable protecting groups for reactive functional groups (such as amino groups in the present disclosure). For the trained practitioner, Greene and Wuts (Protective Groups In Organic Synthesis, Wiley and Sons, 1991) is an authority on this subject. All references cited herein are incorporated herein in their entirety.

The reactions described herein can be monitored according to any suitable method known in the art. For example, product formation can be monitored by broad-spectrum methods such as nuclear magnetic resonance spectroscopy (such as ¹H or ¹³C), infrared spectroscopy, spectrophotometry (such as UV-visible light), mass spectrometry, etc., or by chromatography such as high-performance liquid chromatography (HPLC) or by thin layer chromatography.

### PREPARATION OF INTERMEDIATES

### Intermediate 1: tert-butyl 2-amino-5-(p-toluenesulfonyl)pyrazolo[1,5-a]pyrimidine-3-carboxylate (Compound I1)

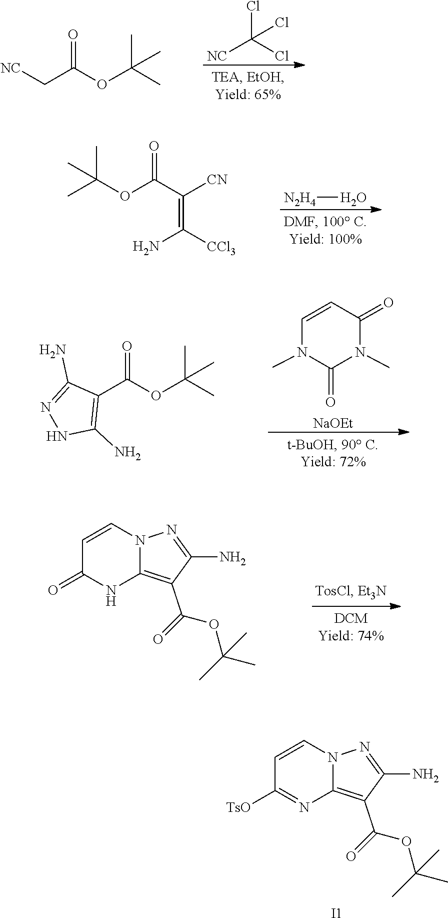

### Step A: (Z)-tert-butyl 3-amino-4,4,4-trichloro-2-cyano-butenoate

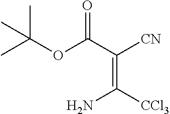

At 0° C., to a solution of tert-butyl cyanoacetate (500 g, 3.5 mol), trichloroacetonitrile (895 g, 6.2 mol) in ethanol (1.5 L). a catalytic amount of triethylamine (30 mL, 0.2 mol) was added dropwise. After the addition was completed, the reaction was carried out at 0° C. for 2 hours, slowly raised to room temperature and reacted for 5 hours. After the reaction was completed, the solvent was concentrated and removed, the residue was stirred and beaten with a mixed solvent (PE/EA=5/1), filtered to obtain the target product (light yellow solid, 580 g, yield 57%). The residue was concentrated, and dissolved in dichloromethane, applied to the column by wet method, and purified bycolumn chromatography to obtain the target product (white solid, 230 g, yield 23%).

¹H NMR (400 MHZ, CDC13) δ 10.20 (brs, ¹H), 6.8 (brs, 1H), 1.55 (s, 9H). m/z=285[M+1]⁺.

### Step B: tert-butyl 3,5-diamino-1H-pyrazole-4-carboxylate

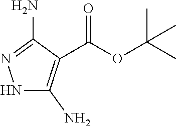

At room temperature, to a solution (1.5 L) of (Z)-tert-butyl 3-amino-4,4,4-trichloro-2-cyano-butenoate (570 g, 2.0 mol) in N,N-dimethylformamide, hydrazine hydrate (375 g, 6.0 mol) was slowly added dropwise. The system exothermic was obvious. The reaction mixture was heated to 100° C., and reacted under stirring for 3 hours. After cooled to room temperature, the system was added with ice water, extracted with ethyl acetate (×4). The organic phases were combined, washed with water (×1), washed with saturated brine (×1), dried over anhydrous sodium sulfate, filtered and concentrated. The residue was dissolved in dichloromethane, applied to the column by wet method, and purified bycolumn chromatography to obtain the target product (white solid, 391 g, yield 99%).

¹H NMR (400 MHZ, DMSO-d₆) δ 5.29 (brs, 2H), 3.39 (brs, 2H), 1.47 (s, 9H). m/z=199[M+1]⁺.

### Step C: tert-butyl 2-amino-5-oxo-4,5-dihydropyrazolo[1,5-a]pyrimidine-3-carboxylate

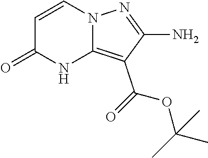 [SMILES: CC(C)(C)OC(=O)c1c(N)nn2ccc(=O)[nH]c12]

At room temperature, to a solution of tert-butyl 3,5-diamino-1H-pyrazole-4-carboxylate (198 g, 1.0 mol) and 1,3-dimethylpyrimidine-2,4(¹H,3H)-dione (140 g, 1.0 mol) in tert-butanol (1.2 L), sodium ethoxide (340 g, 5.0 mol) was slowly added in portions. After the addition was completed, the reaction was raised to 90° C., and reacted for 12 hours. After the reaction was completed, the temperature was lowered to room temperature, and the system was adjusted to a pH value of 6 with 1N hydrochloric acid, extracted with ethyl acetate (×4). The organic phases were combined, washed with water (×1), washed with saturated brine (×1), dried over anhydrous sodium sulfate, filtered and concentrated. The residue was stirred and beaten with a mixed solvent (PE/EA=1/1), and filtered to obtain the target product (yellow solid, 175 g, 70%).

¹H NMR (400 MHz, CDCl₃) δ 7.83 (d, J=8.0 Hz, 1H), 5.95 (d, J=8.0 Hz, 1H), 4.94 (brs, 2H), 1.62 (s, 9H). m /z=251[M+1]⁺.

### Step D: tert-butyl 2-amino-5-(p-toluenesulfonyl)pyrazolo[1,5-a]pyrimidine-3-carboxylate

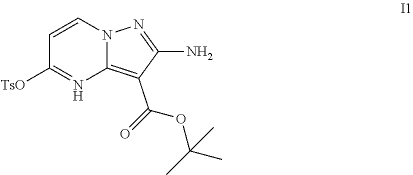 [SMILES: Cc1ccc(S(=O)(=O)Oc2ccn3nc(N)c(C(=O)OC(C)(C)C)c3n2)cc1]

At room temperature, to a solution of tert-butyl 2-amino-5-oxo-4,5-dihydropyrazolo[1,5-a]pyrimidine-3-carboxylate (125 g, 0.5 mol) in N,N-dimethylformamide (500 mL), triethylamine (203 g, 2.0 mol) and p-toluenesulfonyl chloride (115 g, 0.6 mol) were added. After heated to 30° C., the reaction was carried out for 5 hours. After cooled to room temperature, the residue was added with ice water, extracted with ethyl acetate (×3). The organic phases were combined, washed with water (×1), washed with saturated brine (×1), dried over anhydrous sodium sulfate, filtered and concentrated. The residue was dissolved in dichloromethane, applied to the column by wet method, and purified bycolumn chromatography to obtain the target product (pink solid, 152 g, yield 75%).

¹H NMR (400 MHZ, CDCl₃) δ 8.32 (d, J=7.2 Hz, 1H), 8.17 (d, J=8.4 Hz, 2H), 7.35 (d, J=8.0 Hz, 2H), 6.50 (d, J=7.2 Hz, 1H), 5.38 (s, 2H), 2.45 (s, 3H), 1.65 (s, 9H). m/z=405[M+1]⁺.

### Intermediate 2: 3-bromo-2-((ethylamino)methyl)-4-fluorophenol (Compound I2)

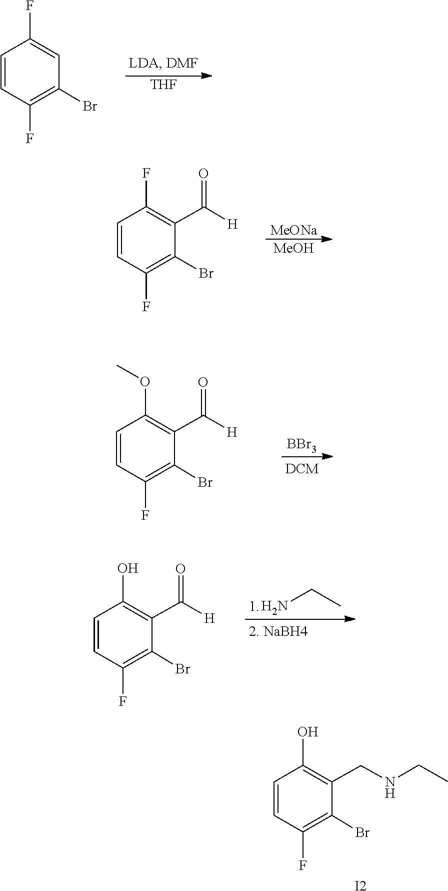

### Step A: 2-bromo-3,6-difluorobenzaldehyde

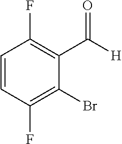 [SMILES: O=Cc1c(F)ccc(F)c1Br]

At −78° C., to a solution of 2,5-difluorobromobenzene (150 g, 0.8 mol) in tetrahydrofuran (1.0 L), lithium diisopropylamide (2M, 500 mL, 1.0 mol) was added, and stirred for 1 hour, then N,N-dimethylformamide (182 g, 2.5 mmol) was added at −78° C. and stirred for 2 hours. After the temperature was slowly raised to 0° C., the reaction mixture was quenched by adding saturated aqueous ammonium chloride solution at 0° C., then diluted by addition of water and extracted with ethyl acetate (x2). The organic phases were combined, washed with water (×1), and washed with saturated brine (×1), dried over anhydrous sodium sulfate, filtered and concentrated. The residue was dissolved in dichloromethane and added to silica gel for dry mixing, and purified by dry column chromatography to obtain the target product (yellow solid, 84 g, yield 49%).

¹H NMR (400 MHZ, CDCl₃) δ 10.31 (s, 1H), 7.36-7.31 (m, 1H), 7.18-7.12 (m, 1H). m/z=221[M+1]⁺.

### Step B: 2-bromo-3-fluoro-6-methoxybenzaldehyde

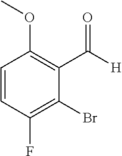

At room temperature, to a solution of 2-bromo-3,6-difluorobenzaldehyde (84 g, 379 mmol) in tetrahydrofuran (200 mL) and methanol (480 mL), sodium methoxide (24.6 g, 455 mmol) was added, followed by heating to 60° C. and stirring for 12 hours. After the reaction was completed as monitored by TLC, it was cooled to room temperature, concentrated under reduced pressure in vacuo to remove most of tetrahydrofuran and methanol. The reaction mixture was quenched by adding water, extracted with ethyl acetate (x2). The organic phases were combined, washed with water (×1), washed with saturated brine (×1), dried over anhydrous sodium sulfate, filtered and concentrated. The residue was dissolved in dichloromethane and added to silica gel for dry mixing, and purified by dry column chromatography to obtain the target product (white solid, 62 g, yield 70%).

¹H NMR (400 MHZ, CDCl₃) δ 10.30 (s, 1H), 7.20 (dd, J=7.6, 9.2 Hz, 1H), 6.86 (dd, J=4.0, 9.2 Hz, 1H), 3.84 (s, 3H). m/z=233[M+1]⁺.

### Step C: 2-bromo-3-fluoro-6-hydroxybenzaldehyde

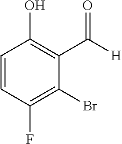 [SMILES: O=Cc1c(O)ccc(F)c1Br]

At -40 ° C., to a solution of 2-bromo-3-fluoro-6-methoxybenzaldehyde (30 g, 129 mmol) in dichloromethane (200 mL), boron tribromide (65 g, 258 mmol) was added dropwise, and then the mixture was stirred at 0° C. for 3 hours. At 0 ° C., the reaction mixture was quenched by adding methanol (40 mL) and saturated sodium bicarbonate solution (100 mL), followed by extraction with ethyl acetate (×2). The organic layer was washed with saturated brine (×1), dried over anhydrous sodium sulfate, filtered and concentrated, and the residue was purified by column chromatography to obtain the target product (23 g, yield 80%) as a yellow solid. ¹H NMR (400 MHZ, CDCl₃) δ 11.78 (s, 1H), 10.35 (s, 1H), 7.32 (dd, J=7.6, 9.2 Hz, 1H), 6.96 (dd, J=4.0, 9.2 Hz, 1H). m/z=219[M+1]⁺.

### Step D: 3-bromo-2-((ethylamino)methyl)-4-fluorophenol

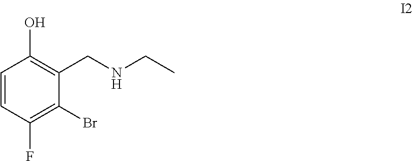

At 0° C., to a solution of 2-bromo-3-fluoro-6-hydroxybenzaldehyde (20 g, 91.3 mmol) in methanol (100 mL) was added a solution of ethylamine in tetrahydrofuran (2M, 91.3 mL, 182.6 mmol); stirring was conducted at 25° C. for 30 minutes, and then sodium borohydride (6.9 g, 182.6 mmol) was added, and the reaction mixture was stirred at 25° C. for 12 hours. The solvent was removed by concentration in vacuo, and the resulting mixture was diluted by addition of water, extracted with ethyl acetate (×2). The organic layer was washed with brine (×1), dried over anhydrous sodium sulfate, filtered and concentrated to give the target product (15.9 g, yield 70%) as a white solid.

¹H NMR (400 MHZ, CDCl₃) δ 6.93 (t, J=8.4 Hz, 1H), 6.71 (dd, J=4.4, 8.4 Hz, 1H), 4.23 (s, 2H), 2.76 (q, J=7.2 Hz, 2H), 1.19 (t, J=7.2 Hz, 3H).

### Intermediate 3: tert-butyl 2-amino-5-chloro-6-fluoropyrazolo[1,5-a]pyrimidine-3-carboxylate (Compound 13)

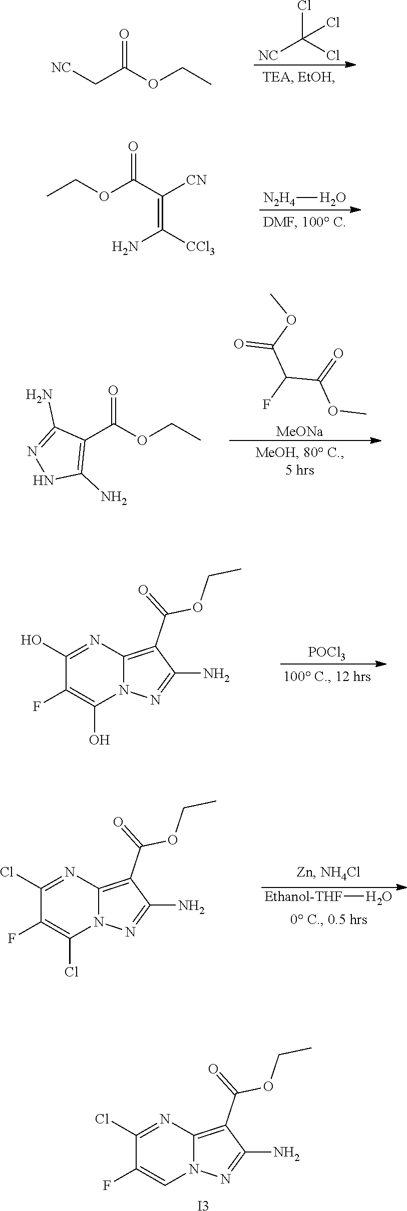

### Step A: (Z)-ethyl 3-amino-4,4,4-trichloro-2-cyano-butenoate

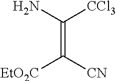

At 0° C., to a solution of ethyl cyanoacetate (41.2 g, 364 mmol) and trichloroacetonitrile (100 g, 693 mmol) in ethanol (120 mL), triethylamine (2.0 g, 20 mmol) was added dropwise. After the addition was completed, the reaction was performed at 0° C. for 2 hours, followed by slowly rising to room temperature and reacting for 30 minutes. After the reaction was completed, the solvent was concentrated and removed, the residue was dissolved in dichloromethane, purified by silica gel column chromatography and eluted with dichloromethane to obtain the target product (93 g, yield 98%).

¹H NMR (400 MHZ, CDCl₃) δ 10.20 (brs, 1H), 6.93 (brs, 1H), 4.30 (q, 2H), 1.33 (t, 3H). m/z=257[M+1]⁺.

### Step B: ethyl 3,5-diamino-1H-pyrazole-4-carboxylate

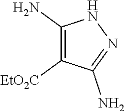 [SMILES: CCOC(=O)c1c(N)n[nH]c1N]

At 0° C., to a solution (300 mL) of (Z)-ethyl 3-amino-4,4,4-trichloro-2-cyano-butenoate (80 g, 311 mmol) in N,N-dimethylformamide, hydrazine hydrate (80%, 58.4 g, 933 mmol) was slowly added dropwise, and the reaction mixture was heated to 100° C. and reacted under stirring for 1.5 hours. The solvent was removed by concentration, and the residue was beaten with dichloromethane, then allowed to stand overnight. The solid was collected by suction filtration, rinsed with dichloromethane, and dried to obtain the target product (35.5 g, 67% yield).

¹H NMR (400 MHZ, DMSO- d₆) δ 10.4 (brs, 1H), 5.35 (brs, 4H), 4.13 (q, 2H), 1.24 (t, 3H). m/z=171[M+1 ]+.

### Step C: ethyl 2-amino-6-fluoro-5,7-dihydroxypyrazolo[1,5-a]pyrimidine-3-carboxylate

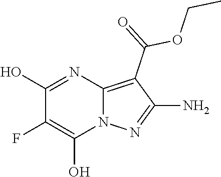 [SMILES: CCOC(=O)c1c(N)nn2c(O)c(F)c(O)nc12]

At room temperature, to a solution of ethyl 3,5-diamino-1H-pyrazole-4-carboxylate (20 g, 118 mmol) in methanol (200 mL), sodium methoxide (31.9 g, 590 mmol) and dimethyl 2-fluoromalonate (28.4 g, 189 mmol) were added, and the reaction mixture was heated to 80° C. and reacted for 5 hours. After the reaction was completed as monitored by TLC, it was cooled to room temperature, concentrated under reduced pressure. The residue was added with water, and adjusted to PH=3 with 2M aqueous hydrochloric acid solution to precipitate a large amount of white solid, which was filtered to obtain the white solid of the title compound (15.1 g, yield 50%).

m/z=257[M+1]⁺.

### Step D: ethyl 2-amino-5,7-dichloro-6-fluoropyrazolo[1,5-a]pyrimidine-3-carboxylate

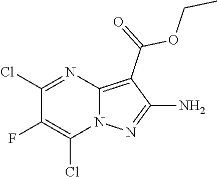 [SMILES: CCOC(=O)c1c(N)nn2c(Cl)c(F)c(Cl)nc12]

At room temperature, ethyl 2-amino-6-fluoro-5,7-dihydroxypyrazolo[1,5-a]pyrimidine-3-carboxylate (15.1 g, 59 mmol) was added to phosphorous oxychloride (200 mL). The reaction mixture was heated to 100° C. and reacted for 12 hours, cooled to room temperature, and concentrated under reduced pressure. The residue was added ice-water mixture, and adjusted to weak alkalinity with saturated aqueous sodium bicarbonate solution, extracted with ethyl acetate (×2). Ethyl acetate phases were combined, washed with saturated sodium chloride solution, dried over anhydrous sodium sulfate, concentrated under reduced pressure, purified by silica gel column and eluted with ethyl acetate/petroleum ether (1/2) to obtain the title compound (6.9 g, yield 40%).

m/z=293[M+1]⁺.

### Step E: ethyl 2-amino-5-chloro-6-fluoropyrazolo[1,5-a]pyrimidine-3-carboxylate

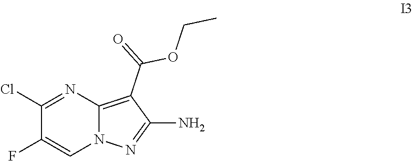 [SMILES: CCOC(=O)c1c(N)nn2cc(F)c(Cl)nc12]

At 0° C., to a mixed solvent of ethyl 2-amino-5,7-dichloro-6-fluoropyrazolo[1,5-a]pyrimidine-3-carboxylate (6.9 g, 23.5 mmol) in ethanol (80 mL), tetrahydrofuran (30 mL) and water (60 mL), activated zinc powder (7.7 g, 117.5 mmol) and ammonium chloride (6.3 g, 117.5 mmol) were added; the reaction mixture was stirred at 0° C. for 0.5 hours, and filtered. The filtrate was diluted by addition of water, extracted with ethyl acetate (×3). The ethyl acetate phases were combined, washed with saturated sodium chloride solution, dried over anhydrous sodium sulfate, concentrated under reduced pressure, and purified by silica gel column to obtain the title compound (3.6 g, yield 60%).

m/z=259[M+1]⁺.

### Intermediate 4: ethyl 2-amino-5-chloro-6-methoxypyrazolo[1,5-a]pyrimidine-3-carboxylate (Compound I4)

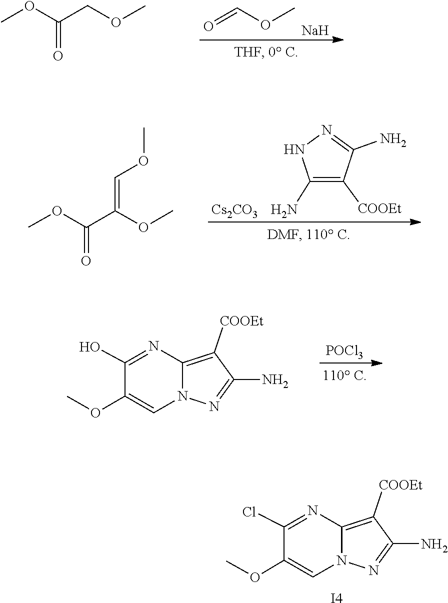

### Step A: (Z)-methyl 2,3-dimethoxyacrylate

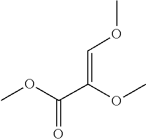

At 0° C., to a solution of methyl 2-methoxyacetate (208g, 2.0 mol) and methyl formate (144 g, 2.4 mol) in tetrahydrofuran (2.0 L), sodium hydride (60% purity, 112 g, 2.8 mol) was added. During the addition process, the reaction system was always kept at a temperature below 0° C., and stirred at 0° C. for 12 hours. It would be observed that a large amount of white solid was generated in the reaction system. The reaction system was added with methyl tert-butyl ether, filtered. The filter cake was vacuum-dried to obtain the target crude product (350 g), which was directly used in the next reaction without purification.

### Step B: ethyl 2-amino-5-hydroxy-6-methoxypyrazolo[1,5-a]pyrimidine-3-carboxylate

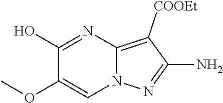 [SMILES: CCOC(=O)c1c(N)nn2cc(OC)c(O)nc12]

At room temperature, a solution of (Z)-methyl 2,3-dimethoxyacrylate (263 g, 1.8 mol) and ethyl 3,5-diamino-1H-pyrazole-4-carboxylate (170 g, 1.0 mol) in N,N-dimethylformamide (2.0 L), cesium carbonate (586 g, 1.8 mol) was added;

the temperature was raised to 110° C. with stirring for 12 hours. After the reaction was completed as monitored by TLC, the reaction system was cooled to room temperature and quenched by adding water, and the reaction solution was diluted. The reaction solution was adjusted to a pH value of about 6 with aqueous hydrochloric acid solution (5 M) to precipitate a large amount of solid, and filtered.

The filter cake was washed with methanol, and vacuum-dried to obtain the target product (177 g, yield 70%).

m/z=253[M+1]⁺.

### Step C: ethyl 2-amino-5-chloro-6-methoxypyrazolo[1,5-a]pyrimidine-3-carboxylate

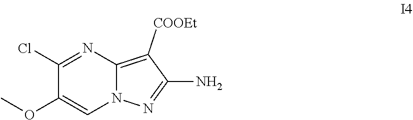 [SMILES: CCOC(=O)c1c(N)nn2cc(OC)c(Cl)nc12]

At room temperature, ethyl 2-amino-5-hydroxy-6-methoxypyrazolo[1,5-a]pyrimidine-3-carboxylate (100 g, 0.4 mol) was added to phosphorus oxychloride (1.0 L). The reaction mixture was warmed up to 110 ° C.and stirred for 12 hours, concentrated under reduced pressure to remove most of the phosphorus oxychloride. The residue was added with ice water, adjusted to weak alkalinity with saturated aqueous sodium carbonate solution, extracted with ethyl acetate (x 3). The ethyl acetate phases were combined, washed with saturated sodium chloride solution, dried over anhydrous sodium sulfate, concentrated under reduced pressure, and purified by silica gel column to obtain the title compound (41 g, yield 39%).

m/z=271[M+1]⁺.

### Intermediate 5: ethyl 2-amino-7-(benzyl(methyl)amino)-5-chloropyrazolo[1,5-a]pyrimidine-3-carboxylate (Compound I5)

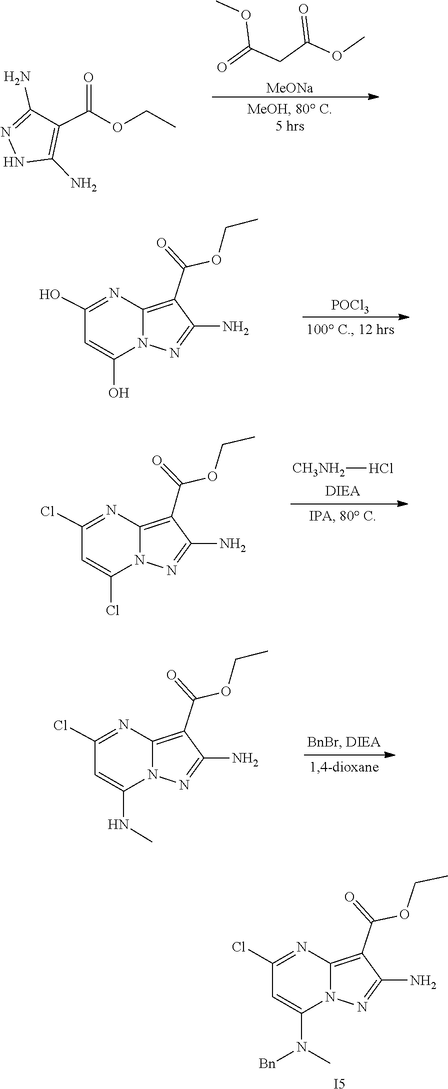

### Step A: ethyl 2-amino-5,7-dihydroxypyrazolo[1,5-a]pyrimidine-3-carboxylate

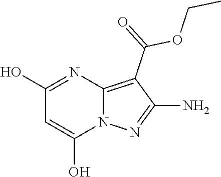 [SMILES: CCOC(=O)c1c(N)nn2c(O)cc(O)nc12]

At room temperature, to a solution of ethyl 3,5-diamino-1H-pyrazole-4-carboxylate (34 g, 0.2 mol) in methanol (200 mL), sodium methoxide (54 g, 1.0 mol) and dimethyl malonate (53 g, 0.4 mol) were added. The reaction mixture was heated to 80 ° C. and reacted for 5 hours. After the reaction was completed as monitored by TLC, it was cooled to room temperature, concentrated under reduced pressure. The residue was added with water, and adjusted to PH=3 with 2M aqueous hydrochloric acid solution to precipitate a large amount of white solid, filtered to obtain the white solid of the title compound (29 g, yield 61%)). m/z=239[M+1]⁺.

### Step B: ethyl 2-amino-5,7-dichloropyrazolo[1,5-a]pyrimidine-3-carboxylate

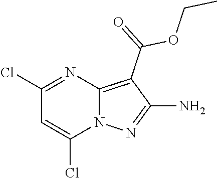 [SMILES: CCOC(=O)c1c(N)nn2c(Cl)cc(Cl)nc12]

At room temperature, ethyl 2-amino-5,7-dihydroxypyrazolo[1,5-a]pyrimidine-3-carboxylate (10.7 g, 44.8 mmol) was added to phosphorus oxychloride (150 mL). The reaction mixture was warmed up to 110° C. and stirred for 12 hours, concentrated under reduced pressure to remove most of the phosphorus oxychloride. The residue was added with ice water, and adjusted to weak alkalinity with saturated aqueous sodium carbonate solution, extracted with ethyl acetate (×3). The ethyl acetate phases were combined, washed with saturated sodium chloride solution, dried over anhydrous sodium sulfate, concentrated under reduced pressure, and purified by silica gel column to obtain the title compound (6.2 g, yield 50%).

m/z=275[M+1]⁺.

### Step C: ethyl 2-amino-5-chloro-7-(methylamino)pyrazolo[1,5-a]pyrimidine-3-carboxylate

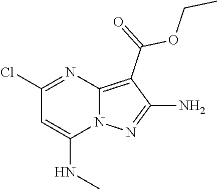 [SMILES: CCOC(=O)c1c(N)nn2c(NC)cc(Cl)nc12]

At room temperature, to a solution of ethyl 2-amino-5,7-dichloropyrazolo[1,5-a]pyrimidine-3-carboxylate (5.3 g, 19.2 mmol) and N,N-diisopropylethylamine (7.4 g, 57.6 mmol) in isopropanol (50 mL), methylamine hydrochloride (7.4 g, 57.6 mmol) was added. The reaction mixture was heated to 80 ° C.and reacted for 5 hours. After the reaction was completed as monitored by TLC, it was cooled to room temperature, concentrated under reduced pressure. The residue was added with water, and extracted with ethyl acetate (×3). The ethyl acetate phases were combined, washed with saturated sodium chloride solution, and dried over anhydrous sodium sulfate, concentrated under reduced pressure and purified by silica gel column to obtain the title compound (4.7 g, yield 90%).

m/z=270[M+1]⁺.

### Step D: ethyl 2-amino-7-(benzyl(methyl)amino)-5-chloropyrazolo[1,5-a]pyrimidine-3-carboxylate

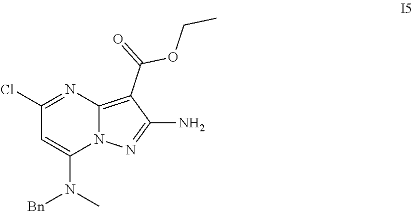 [SMILES: CCOC(=O)c1c(N)nn2c(N(C)Cc3ccccc3)cc(Cl)nc12]

At room temperature, to a solution of 2-amino-5-chloro-7-(methylamino)pyrazolo[1,5-a]pyrimidine-3-carboxylate (4.2 g, 15.7 mmol) and

N,N-diisopropylethylamine (6.1 g, 47.1 mmol) in N,N-dimethylformamide (30 mL), benzyl bromide (5.4 g, 31.4 mmol) was added. The reaction was conducted at room temperature for 8 hours. After the reaction was completed as monitored by TLC, it was cooled to room temperature, concentrated under reduced pressure. The residue was added with water, and extracted with ethyl acetate (×3). The ethyl acetate phases were combined, washed with saturated sodium chloride solution, dried over anhydrous sodium sulfate, concentrated under reduced pressure, purified by silica gel column to obtain the title compound (5.3 g, yield 93%).

m/z=360[M+1]⁺.

### Intermediate 6: methyl 2-amino-6-chloroimidazo[1,2-b]pyridazine-3-carboxylate (Compound 16)

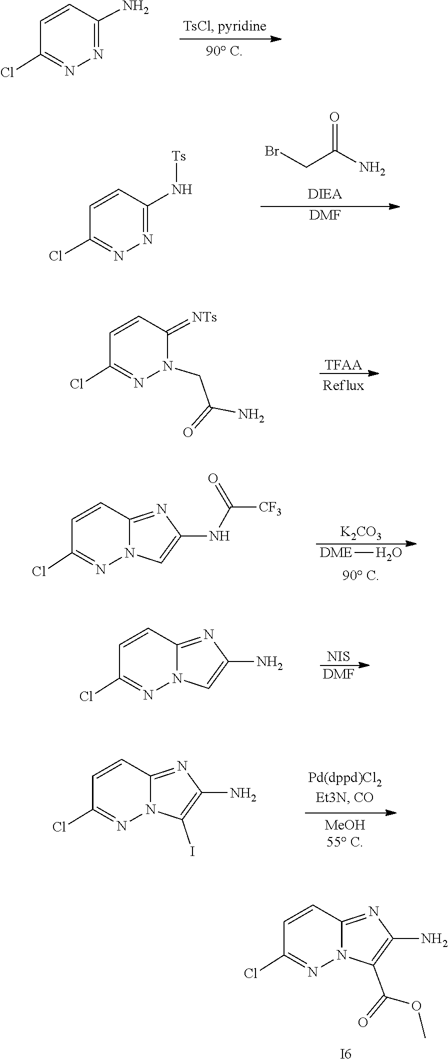

Methyl 2-amino-6-chloroimidazo[1,2-b]pyridazine-3-carboxylate (Compound 16) was synthesized according to the method of Example 45 intermediate in patent WO2017079519A1.

### Intermediate 7: (R)-propyl 2-hydroxypivalate (Compound I7)

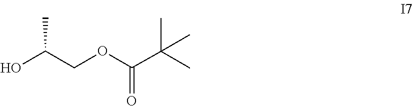

At 0° C., to a solution of (R)-propane-1,2-diol (76 g, 1.0 mol) and triethylamine (122g, 1.2 mol) in dichloromethane (1.0 L), pivaloyl chloride (121 g, 1.0 mol) was slowly added dropwise. The reaction mixture was raised to room temperature and reacted under stirring overnight. After the reaction was completed as monitored by TLC, it was cooled to room temperature, concentrated under reduced pressure. The residue was added with water, and extracted with ethyl acetate (×3). Ethyl acetate phases were combined, washed with saturated sodium chloride solution, dried over anhydrous sodium sulfate, concentrated under reduced pressure, and purified by silica gel column to obtain the title compound (128 g, yield 80%).

### Intermediate 8: (S)-2-(3-bromo-2-((ethylamino)methyl)-4-fluorophenoxy) propan-1-amine (Compound 18)

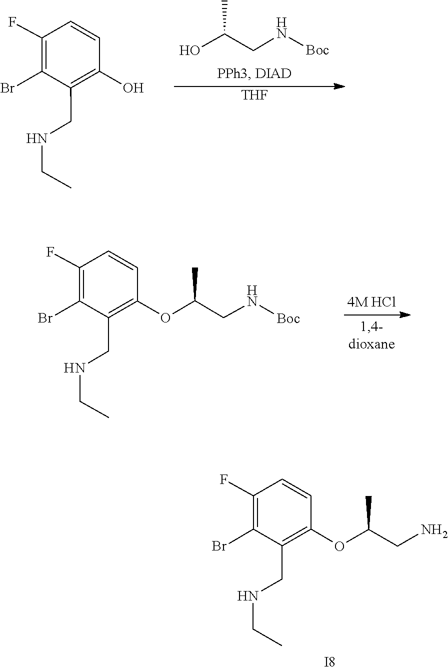

### Step A: (S)-tert-butyl (2-(3-bromo-2-((ethylamino)methyl)-4-fluorophenoxy) propyl)carbamate

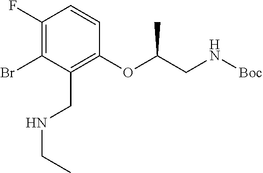 [SMILES: CCNCc1c(O[C@@H](C)CNC(=O)OC(C)(C)C)ccc(F)c1Br]

At 0° C., to a solution of 3-bromo-2-((ethylamino)methyl)-4-fluorophenol (Intermediate 12) (5 g, 20 mmol), triphenylphosphine (7.9 g, 30 mmol) and (R)-tert-butyl (2-hydroxypropyl)carbamate (5.3 g, 30 mmol) in tetrahydrofuran (60 mL), diisopropyl azodicarboxylate (8.1 g, 40 mmol) was slowly added; the reaction mixture was raised to room temperature and reacted under stirring for 6 hours. The progress of the reaction was monitored by TLC, and the reaction was basically completed. The reaction was cooled to room temperature, concentrated under reduced pressure. The residue was added with water, extracted with ethyl acetate (×3). The ethyl acetate phases were combined, washed with saturated sodium chloride solution (×1), dried over anhydrous sodium sulfate, concentrated under reduced pressure and purified by silica gel column to obtain the title compound (6.1 g, yield 75%).

m/z=405[M+1]⁺.

### Step B: (S)-2-(3-bromo-2-((ethylamino)methyl)-4-fluorophenoxy)propan-1-amine

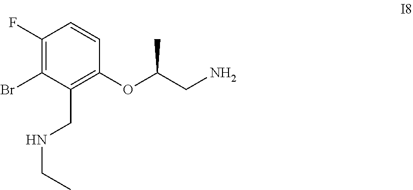 [SMILES: CCNCc1c(O[C@@H](C)CN)ccc(F)c1Br]

At 0° C., to a solution of (S)-tert-butyl (2-(3-bromo-2-((ethylamino)methyl)-4-fluorophenoxy)propyl)carbamate (6.0 g, 14.8 mmol) in dichloromethane (50 mL), 4M HCl (19 mL) in 1,4-dioxane was slowly added dropwise. The resulting mixture was stirred at 30° C.for 2 hours to precipitate a large amount of white solid. The progress of the reaction was monitored by TLC and the reaction was complete. The reaction mixture was concentrated under reduced pressure to remove most of the solvent and hydrochloric acid gas. The residue was diluted by addition of water, adjusted to alkalinity with saturated aqueous sodium carbonate solution, extracted with ethyl acetate (×3). The ethyl acetate phases were combined, washed with saturated sodium chloride solution (×1), dried over anhydrous sodium sulfate, concentrated under reduced pressure, and purified by silica gel column to obtain the title compound (4.5 g, yield 90%). m/z=305[M+1]⁺.

### Intermediate 9: (R)-tert-butvl (3-hydroxybutyl)carbamate (Compound 19)

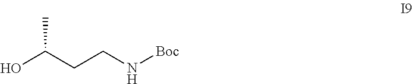

(R)-tert-butyl (3-hydroxybutyl)carbamate (Compound 19) was synthesized according to the intermediate synthesis method in Example 4 of patent WO2020001415A1.

### Intermediate 10: (S)-3-(3-bromo-2-((ethylamino)methyl)-4-fluorophenoxy) butane-1-amine (Compound 110)

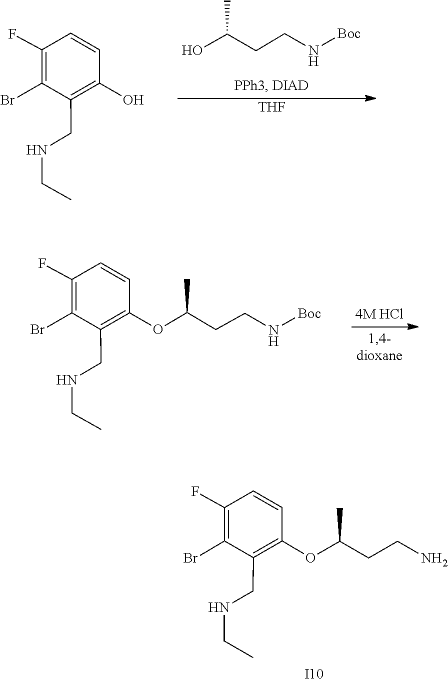

The synthesis was carried out according to the synthesis method of Intermediate 8 (Compound 18), except that replacing (R)-tert-butyl (2-hydroxypropyl)carbamate with (R)-tert-butyl (3-hydroxybutyl)carbamate.

### Intermediate 11: (R)-2-(4-hydroxypentyl)isoindole-1,3-dione (Compound I11)

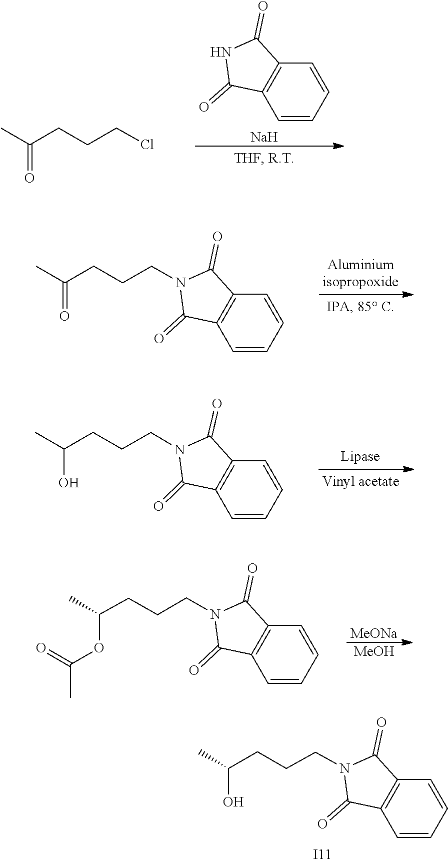

### Step A: 2-(4-oxopentyl)isoindole-1,3-dione

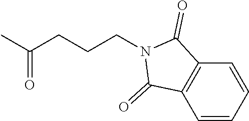 [SMILES: CC(=O)CCCN1C(=O)c2ccccc2C1=O]

At 0° C., sodium hydride (8.0 g, 60%, 0.2 mol) was slowly added in batches to a solution of phthalimide (29.4 g, 0.2 mol) in dry tetrahydrofuran (300 mL). After the addition was completed, stirring was continued at 0° C. for 0.5 hours, followed by adding 5-chloro-2-pentanone (24.2 g, 0.2 mol), slowly warming to room temperature and stirring for 6 hours. The reaction mixture was concentrated under reduced pressure to remove most of the solvent, diluted by addition of water, extracted with ethyl acetate (×3). The ethyl acetate phases were combined, washed with saturated sodium chloride solution (×1), dried over anhydrous sodium sulfate, concentrated under reduced pressure, and purified by silica gel column to obtain the title compound (40 g, yield 86%).

¹H NMR (400 MHZ, CDCl₃) δ 7.85 (m, 2H), 7.72 (m, 2H), 3.72 (t, J=6.6 Hz, 2H), 2.51 (t, J=7.0 Hz, 2H), 2.15 (s, 3H), 1.97 (m, 2H). m/z=232[M+1]⁺.

### Step B: 2-(4-hydroxypentyl)isoindole-1,3-dione

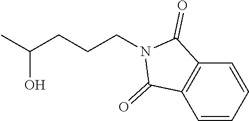 [SMILES: CC(O)CCCN1C(=O)c2ccccc2C1=O]

At room temperature, to a solution of 2-(4-oxopentyl)isoindoline-1,3-dione (40 g, 173 mmol) in isopropanol (300 mL), isopropanol aluminum (88.4 g, 433 mmol) was slowly added portionwise, refluxing for 4 hours. Most of the solvent was removed under reduced pressure, and 1M hydrochloric acid was added to neutralize, followed by extraction with ethyl acetate (×3). The ethyl acetate phases were combined, washed with saturated sodium chloride solution (×1), dried over anhydrous sodium sulfate, concentrated under reduced pressure and purified by silica gel column to obtain the title compound (20 g, yield 50%).

m/z=234[M+1]⁺.

### Step C: (R)-5-(1,3-dioxoisoindol-2-yl)pentan-2-yl acetate

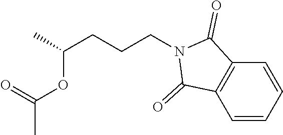 [SMILES: CC(=O)O[C@H](C)CCCN1C(=O)c2ccccc2C1=O]

At room temperature, 2-(4-hydroxypentyl)isoindole-1,3-dione (20 g, 85.7 mmol), vinyl acetate (44.3 g, 514 mmol) and lipase (8.2 g, 34.3 mmol) were added into isopropyl ether (500 mL), stirred overnight at room temperature, filtered through diatomaceous earth, concentrated to remove isopropyl ether, diluted by addition of water, extracted with ethyl acetate (×3). The ethyl acetate phases were combined, and washed with saturated sodium chloride solution (×1), dried over anhydrous sodium sulfate, concentrated under reduced pressure, and purified by silica gel column to obtain the title compound (11 g, yield 47%).

m/z=276[M+1]⁺.

### Step D: (R)-2-(4-hydroxypentyl)isoindole-1,3-dione

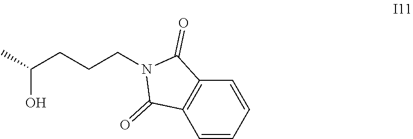 [SMILES: C[C@@H](O)CCCN1C(=O)c2ccccc2C1=O]

At 0° C., to a solution of (R)-5-(1,3-dioxoisoindol-2-yl)pentane-2-yl acetate (5 g, 18.2 mmol) in methanol (50 mL), sodium methoxide (1.2 g, 21.8 mmol) was added, and the reaction was raised to room temperature and reacted for 3 hours. The reaction progress was monitored by TLC, and the reaction was completed. Most of the solvent were removed by concentration under reduced pressure. The residue was diluted by addition of water, extracted with ethyl acetate (×3). The ethyl acetate phases were combined, washed with saturated sodium chloride solution (×1), dried over anhydrous sodium sulfate, concentrated under reduced pressure, and purified by silica gel column to obtain the title compound (3.8 g, yield 89%).

m/z=234[M+1]⁺.

### Intermediate 12: (S)-4-(3-bromo-2-((ethylamino)methyl)-4-fluorophenoxy) pentan-1-amine (Compound I12)

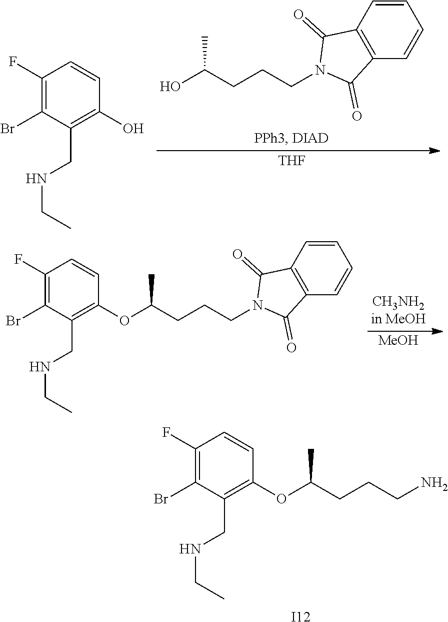

### Step A: (S)-2-(4-(3-bromo-2-((ethylamino)methyl)-4-fluorophenoxy)pentyl) isoindole-1,3-dione

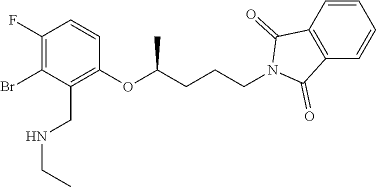 [SMILES: CCNCc1c(O[C@@H](C)CCCN2C(=O)c3ccccc3C2=O)ccc(F)c1Br]

The synthesis was carried out according to the synthesis method of step A of Intermediate 8 (Compound 18), except replacing (R)-tert-butyl (2-hydroxypropyl) carbamate with (R)-2-(4-hydroxypentyl)isoindole-1,3-dione.

m/z=463[M+1]⁺.

### Step B: (S)-4-(3-bromo-2-((ethylamino)methyl)-4-fluorophenoxy)pentan-1-amine

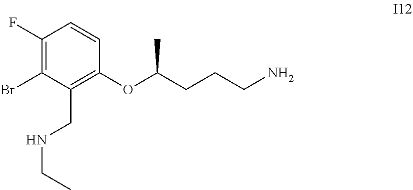 [SMILES: CCNCc1c(O[C@@H](C)CCCN)ccc(F)c1Br]

At room temperature, to a solution of (S)-2-(4-(3-bromo-2-((ethylamino)methyl)-4-fluorophenoxy)pentyl)isoindole-1,3-dione (5 g, 10.8 mmol) in methanol (50 mL), methanol solution (30 mL, 33wt %) was added, refluxing for 4 hours. The solvent was removed under reduced pressure, and the residue was separated by silica gel column chromatography to obtain the target product (2.9 g, yield 81%).

m/z=333[M+1]⁺.

### Intermediate 13: (S)-3-((1-aminopropan-2-yl)oxy)-2-((ethylamino)methyl)-6-fluorobenzonitrile (Compound I13)

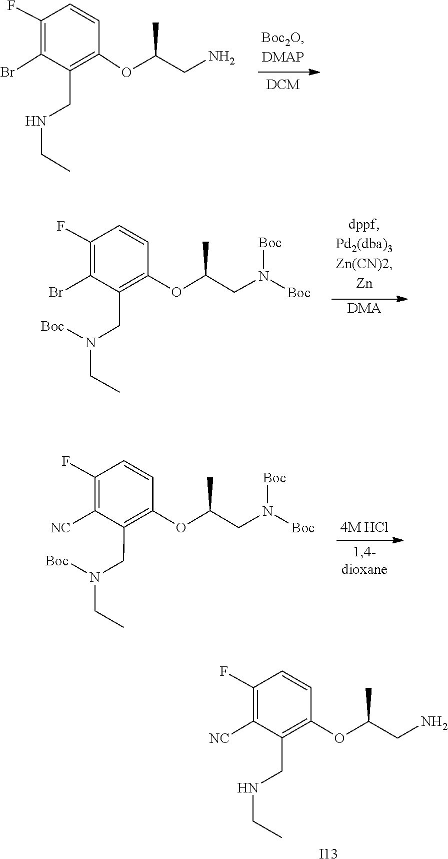

### Step A: (S)-tert-butyl (2-(3-bromo-2-(((tert-butoxycarbonyl)(ethyl)amino) methyl)-4-fluorophenoxy)propyl)(tert-butoxycarbonyl)carbamate

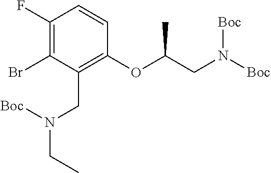 [SMILES: CCN(Cc1c(O[C@@H](C)CN(C(=O)OC(C)(C)C)C(=O)OC(C)(C)C)ccc(F)c1Br)C(=O)OC(C)(C)C]

At room temperature, to a solution of (S)-2-(3-bromo-2-((ethylamino)methyl)-4-fluorophenoxy)propan-1-amine (5 g, 16.4 mmol) in dichloromethane (40 mL), di-tert-butyl dicarbonate (21.5 g, 98.4 mmol) and 4-dimethylaminopyridine (1.0 g, 8.2 mmol) were added; the reaction was carried out at room temperature for 6 hours. The reaction progress was monitored by TLC, and the reaction was complete. Most of the solvent was removed by concentration under reduced pressure. The residue was diluted by addition of water, extracted with ethyl acetate (×3). The ethyl acetate phases were combined, washed with saturated sodium chloride solution (×1), dried over anhydrous sodium sulfate, and concentrated under reduced pressure, and purified by silica gel column to obtain the title compound (9.2 g, yield 93%).

m/z=605[M+1]⁺.

### Step B: (S)-tert-butyl (tert-butoxycarbonyl)(2-(2-(((tert-butoxycarbonyl) (ethyl)amino) methyl)-3-cyano-4-fluorophenoxy)propyl)carbamate

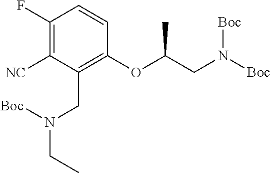 [SMILES: CCN(Cc1c(O[C@@H](C)CN(C(=O)OC(C)(C)C)C(=O)OC(C)(C)C)ccc(F)c1C#N)C(=O)OC(C)(C)C]

At room temperature, to a degassed mixture of (S)-tert-butyl (2-(3-bromo-2-(((tert-butoxycarbonyl)(ethyl)amino)methyl)-4-fluorophenoxy)propyl)(tert- butoxycarbonyl)carbamate (9.0 g, 14.9 mmol), zinc cyanide (5.2 g, 44.7 mmol), zinc powder (195 mg, 3.0 mmol) and 1,1′-bis(diphenylphosphino)dicene iron (3.3 g, 6.0 mmol) in N,N-dimethylacetamide (100 mL), tris(dibenzylideneacetone) dipalladium (2.7 g, 3.0 mmol) was added. The mixture was heated to 130° C. and reacted with stirring for 3 hours. The progress of the reaction was monitored by TLC and the reaction was completed. The reaction mixture was filtered through diatomaceous earth, washed with ethyl acetate (×3), washed with water (×1), washed with saturated sodium chloride solution (×1), dried over anhydrous sodium sulfate, concentrated under reduced pressure, and purified by silica gel column to obtain the title compound (6.5 g, yield 79%).

m/z=552[M+1]⁺.

### Step C: (S)-3-((1-aminopropan-2-yl)oxy)-2-((ethylamino)methyl)-6-fluorobenzonitrile

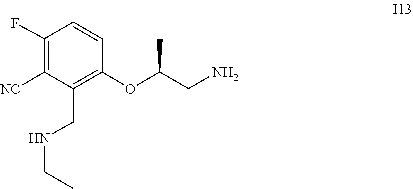 [SMILES: CCNCc1c(O[C@@H](C)CN)ccc(F)c1C#N]

At 0° C., to a solution of (S)-tert-butyl (tert-butoxycarbonyl)(2-(2-(((tert-butoxycarbonyl) (ethyl)amino)methyl) -3-cyano-4-fluorophenoxy)propyl) carbamate (6.3 g, 11.4 mmol) in dichloromethane (50 mL), 4M HCl in 1,4-dioxane (14 mL) was slowly added dropwise. The resulting mixture was stirred at 30° C. for 2 hours to precitipate a large amount of white solid. The progress of the reaction was monitored by TLC and the reaction was complete. Most of the solvent and hydrochloric acid gas was removed by concentration under reduced pressure. The the residue was diluted by addition of water, adjusted to alkalinity with saturated aqueous sodium carbonate, extracted with ethyl acetate (×3). The ethyl acetate phases were combined, and washed with saturated sodium chloride solution (×1), dried over anhydrous sodium sulfate, concentrated under reduced pressure, and purified by silica gel column to obtain the title compound (2.7 g, yield 94%).

m/z=252[M+1]⁺.

### Intermediate 14: (S)-3-((4-aminotert-butyl-2-yl)oxy)-2-((ethylamino)methyl)-6-fluorobenzonitrile (Compound I14)

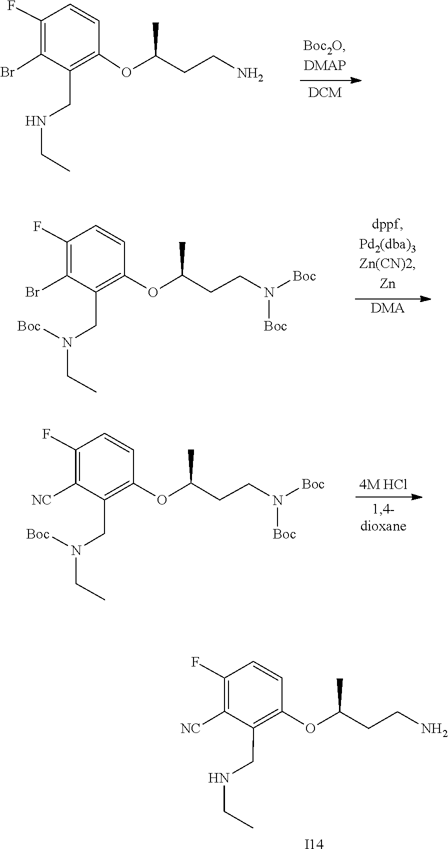

The synthesis was carried out according to the synthesis method of Intermediate 13 (Compound I13), except that replacing (S)-2-(3-bromo-2-((ethylamino)methyl)-4-fluorophenoxy)propane-1-amine with (S)-3-(3- bromo-2-((ethylamino) methyl)-4-fluorophenoxy)butane-1-amine.

Intermediate 15: (S)-3-((5-aminopentan-2-yl)oxy)-2-((ethylamino)methyl)-6-fluorobenzonitrile (Compound I15)

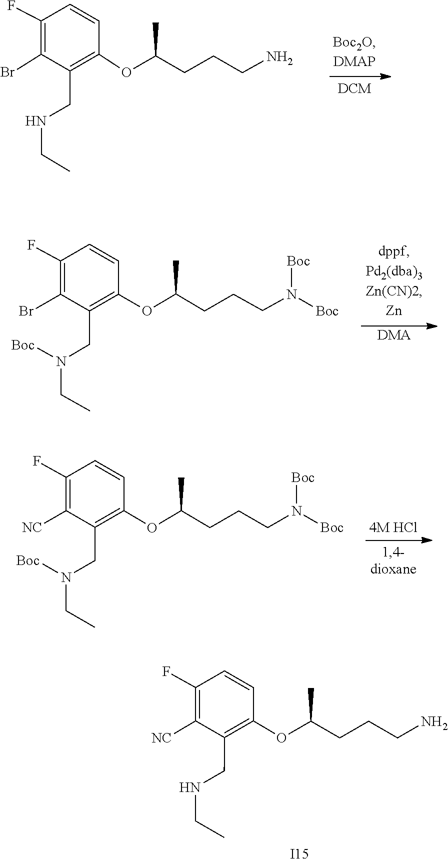

The synthesis was carried out according to the synthesis method of Intermediate 13 (Compound I13), except that replacing (S)-2-(3-bromo-2-((ethylamino)methyl)-4-fluorophenoxy)propane-1-amine with (S)-4-(3- bromo-2-((ethylamino)methyl)-4-fluorophenoxy)pentane-1-amine.

### Example 1 (S, 1³E, 1⁴E)-2-ethyl-45-fluoro-6-methyl-9-oxo-5,8-dioxa-2-aza-1(5,3)-pyrazolo[1,5-a]pyrimidine-4(1,2)-benzocyclononane-4⁶-carbonitrile

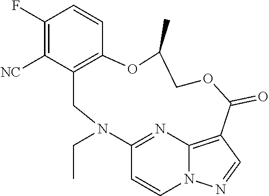 [SMILES: CCN1Cc2c(ccc(F)c2C#N)O[C@@H](C)COC(=O)c2cnn3ccc1nc23]

### Step A: ethyl 5-((2-bromo-3-fluoro-6-hydroxybenzyl)(ethyl)amino) pyrazolo[1,5-a]pyrimidine-3-carboxylate

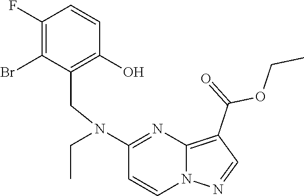 [SMILES: CCOC(=O)c1cnn2ccc(N(CC)Cc3c(O)ccc(F)c3Br)nc12]

At room temperature, a reactor was charged with ethyl 5-chloropyrazolo[1,5-a]pyrimidine-3-carboxylate (2.3 g, 10 mmol), 3-bromo-2-((ethylamino)methyl)-4-fluorophenol (2.5 g, 10 mmol), N,N-diisopropylethylamine (2.6 g, 20 mmol) and n-butanol (30 mL). The resulting mixture was heated at 95° C. for 12 hours. The progress of the reaction was monitored by TLC, and the reaction was basically completed. The reaction was cooled to room temperature, concentrated under reduced pressure. The residue was added with water, extracted with ethyl acetate (×3). The ethyl acetate phases were combined, washed with saturated sodium chloride solution (×1), dried over anhydrous sodium sulfate, concentrated under reduced pressure and purified by silica gel column to obtain the title compound (3.8 g, yield 87%).

m/z=437[M+1]⁺.

### Step B: (S)-ethyl 5-((2-bromo-3-fluoro-6-((1-pivaloyloxy)propan-2-yl)oxy) benzyl)(ethyl)amino)pyrazolo[1,5-a]pyrimidine-3-carboxylate

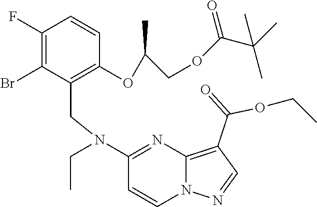 [SMILES: CCOC(=O)c1cnn2ccc(N(CC)Cc3c(O[C@@H](C)COC(=O)C(C)(C)C)ccc(F)c3Br)nc12]

At 0° C., to a solution of ethyl 5-((2-bromo-3-fluoro-6-hydroxybenzyl)(ethyl) amino)pyrazolo[1,5-a]pyrimidine-3-carboxylate (2.0 g, 4.6 mmol), triphenylphosphine (1.8 g, 6.9 mmol) and (R)-propyl 2-hydroxypivalate (0.9 g, 5.5 mmol) in tetrahydrofuran (20 mL), diisopropyl azodicarboxylate (1.4 g, 6.9 mmol) were slowly added; the reaction mixture was raised to room temperature and reacted with stirring overnight. The reaction progress was monitored by TLC, and the reaction was basically completed. The reaction was cooled to room temperature, concentrated under reduced pressure. The residue was added with water, extracted with ethyl acetate (×3). The ethyl acetate phases were combined, washed with saturated sodium chloride solution (×1), dried over anhydrous sodium sulfate, concentrated under reduced pressure and purified by silica gel column to obtain the title compound (1.8 g, yield 68%).

m/z=579[M+1]⁺.

### Step C: (S)-5-((2-bromo-3-fluoro-6-((1-hydroxypropan-2-yl)oxy)benzyl) (ethyl)amino)pyrazolo[1,5-a]pyrimidine-3-carboxylic acid

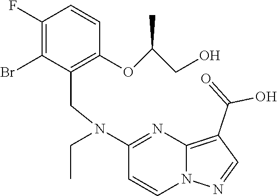 [SMILES: CCN(Cc1c(O[C@@H](C)CO)ccc(F)c1Br)c1ccn2ncc(C(=O)O)c2n1]

At 0° C., to a solution of (S)-ethyl 5-((2-bromo-3-fluoro-6-((1-pivaloyloxy) propan-2-yl)oxy)benzyl)(ethyl)amino)pyrazolo[1,5-a]pyrimidine-3-carboxylate (1.8 g, 3.1 mmol) in ethanol (80 mL) and water (20 mL), lithium hydroxide monohydrate (2.6 g, 62.0 mmol) was slowly added. The resulting mixture was refluxed overnight at 95° C. The disappearance of raw materials was monitored by LCMS, and the reaction was completed. The resulting mixture was acidified with 4 N HCl, extracted with ethyl acetate (×2). The ethyl acetate phases were combined, washed with saturated sodium chloride solution (×1), dried over anhydrous sodium sulfate, concentrated under reduced pressure, and purified by silica gel column to obtain the title compound (1.3 g, yield 90%).

m/z=467[M+1]+.

### Step D: (S, 1³E, 1⁴E)-4⁶-bromo-2-ethyl-4⁵-fluoro-6-methyl-5,8-dioxa-2-aza-1(5,3)-pyrazolo[1,5-a]pyrimidine-4(1,2)-benzocyclononan-9-one

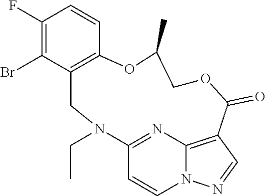 [SMILES: CCN1Cc2c(ccc(F)c2Br)O[C@@H](C)COC(=O)c2cnn3ccc1nc23]

At room temperature, a reactor was charged with (S)-5-((2-bromo-3-fluoro-6-((1-hydroxypropan-2-yl)oxy)benzyl)(ethyl)amino)pyrazolo[1,5-a]pyrimidine-3-carboxylic acid (3.0 g, 6.4 mmol), 1-(3-dimethylaminopropyl)-3-ethylcarbodiimide hydrochloride (1.84 g, 9.6 mmol), 4-dimethylaminopyridine (1.0 g, 8.4 mmol) and dichloromethane (200 mL). The resulting mixture was refluxed overnight at 40° C. The disappearance of raw materials was monitored by LCMS and the reaction was completed. Dichloromethane was removed in vacuo and the residue was purified by silica gel column to obtain the title compound (980 mg, yield 34%).

¹H NMR (400 MHZ, DMSO-d₆) δ 8.65 (d, J=7.8 Hz, 1H), 8.13 (s, 1H), 7.13-7.07 (m, 2H), 6.79 (d, J=7.9 Hz, 1H), 5.68 (dd, J=14.2, 1.5 Hz, 1H), 4.96-4.83 (m, 1H), 4.53 (dd, J=12.0, 3.6 Hz, 1H), 4.16-4.05 (m, 2H), 3.86-3.77 (m, 2H), 1.46 (d, J=6.4 Hz, 3H), 1.18 (t, J=7.0 Hz, 3H).

m/z=449[M+1]+.

### Step D: (S, 1³E, 1⁴E)-2-ethyl-4⁵-fluoro-6-methyl-9-oxo-5,8-dioxa-2-aza-1(5,3)-pyrazolo[1,5-a]pyrimidine-4(1,2)-benzocyclononane-4⁶-carbonitrile

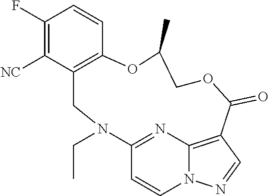 [SMILES: CCN1Cc2c(ccc(F)c2C#N)O[C@@H](C)COC(=O)c2cnn3ccc1nc23]

At room temperature, a reactor was charged with (S, 1³E, 1⁴E)-4⁶-bromo-2-ethyl-4⁵-fluoro-6-methyl-5,8-dioxa-2-aza-1(5,3)-pyrazolo[1,5-a]pyrimidine-4(1,2) -benzocyclononan-9-one (1.0 g, 2.2 mmol), cyanide copper (0.6 g, 6.6 mmol) and N,N-dimethylacetamide (10 mL). The resulting mixture was heated at 140° C.under protection of nitrogen for 8 hours. The disappearance of raw materials was monitored by LCMS and the reaction was completed. The reaction mixture was cooled to room temperature, dichloromethane was added to the reaction mixture for dilution. An appropriate amount of diatomaceous earth, an appropriate amount of activated carbon, and an excess buffer (H₂O/NH₄Cl/NH₄ ClOH=9.4/4.0/3.8) were added to the filtrate for filtering. The reaction mixture was stirred at room temperature for 2 hours. The reaction mixture was filtered through one inch of diatomaceous earth bed and washed with DCM (×3). The layers were separated, and the organic layer was washed with buffer (×2) and water (×2). The organic layer was concentrated to the minimum volume and purified by silica gel column to obtain the title compound (760 mg, yield 83%).

¹H NMR (400 MHZ, CDCl₃) δ 8.31 (dd, J=7.8, 4.5 Hz, 1H), 8.23 (d, J=10.4 Hz, 1H), 7.08-6.97 (m, 2H), 6.55 (dd, J=7.8, 3.7 Hz, 1H), 5.91-5.81 (m, 1H), 5.45-5.36 (m, 0.4H), 4.85-4.76 (m, 0.6H), 4.71 (dd, J=11.8, 3.8 Hz, 0.6H), 4.39 (dd, J=11.5, 5.4 Hz, 0.4H), 4.34-4.23 (m, 1H), 4.21-4.09 (m, 1H), 3.99-3.80 (m, 2H), 1.68-1.58 (m, 3H), 1.31-1.24 (m, 3H). m/z=396[M+1]⁺.

### Example 2 (S, 1³E, 1⁴E)-1²-amino-2-ethyl-4⁵-fluoro-6-methyl-9-oxo-5,8-dioxa-2-aza-1(5,3)-pyrazolo[1,5-a]pyrimidine-4(1,2)-benzocyclononane-4⁶ carbonitrile

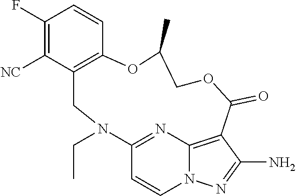 [SMILES: CCN1Cc2c(ccc(F)c2C#N)O[C@@H](C)COC(=O)c2c(N)nn3ccc1nc23]

### Step A: tert-butyl 2-amino-5-((2-bromo-3-fluoro-6-hydroxybenzyl) (ethyl)amino)pyrazolo[ 1,5-a]pyrimidine-3-carboxylate

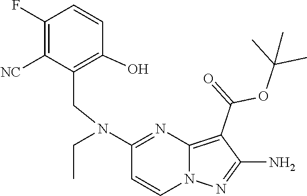 [SMILES: CCN(Cc1c(O)ccc(F)c1C#N)c1ccn2nc(N)c(C(=O)OC(C)(C)C)c2n1]

At room temperature, a reactor was charged with tert-butyl 2-amino-5-(p-toluenesulfonyl)pyrazolo[1,5-a]pyrimidine-3-carboxylate (40.5 g, 100 mmol), 3-bromo-2-((ethylamino)methyl)-4-fluorophenol (29.8 g, 120 mmol), N,N-diisopropylethylamine (38.8 g, 300 mmol) and n-butanol (400 mL). The resulting mixture was heated at 95° C. for 12 hours. The progress of the reaction was monitored by TLC, and the reaction was basically completed. The reaction was cooled to room temperature, concentrated under reduced pressure. The residue was added with water, extracted with ethyl acetate (×3). The ethyl acetate phases were combined, washed with saturated sodium chloride solution (×1), dried over anhydrous sodium sulfate, concentrated under reduced pressure and purified by silica gel column to obtain the title compound (33.6 g, yield 70%).

m/z=480[M+1]⁺.

### Step B: tert-butyl 2-amino-5-((2-cyano-3-fluoro-6-hydroxybenzyl)(ethyl) amino)pyrazolo[1,5-a]pyrimidine-3-carboxylate

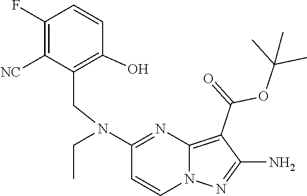 [SMILES: CCN(Cc1c(O)ccc(F)c1C#N)c1ccn2nc(N)c(C(=O)OC(C)(C)C)c2n1]

At room temperature, a reactor was charged with tert-butyl 2-amino-5-((2-bromo-3-fluoro-6-hydroxybenzyl)(ethyl)amino)pyrazolo[1,5-a]pyrimidine-3-formate (24 g, 50 mmol), cuprous cyanide (11.2 g, 125 mmol) and N,N-dimethylacetamide (120 mL). The resulting mixture was heated at 100° C. for 96 hours. The progress of the reaction was monitored by TLC, and the reaction was basically completed. The reaction was cooled to room temperature, dichloromethane was added to the reaction mixture for dilution. An appropriate amount of diatomaceous earth, an appropriate amount of activated carbon, and an excess buffer (H₂O/NH₄Cl/NH₄OH=9.4/4.0/3.8) were chareged into the filtrate for filtering. The reaction mixture was stirred at room temperature for 2 hours. The reaction mixture was filtered through one inch of diatomaceous earth bed and washed with DCM (×3). The layers were separated, and the organic layer was washed with buffer (×2) and water (×2). The organic layer was concentrated to the minimum volume and purified by silica gel column to obtain the title compound (19.2 g, yield 90%).

m/z=427[M+1]⁺.

### Step C: (S)-tert-butyl 2-amino-5-((2-cyano-3-fluoro-6-((1-pivaloyloxy) propan-2-yl)oxy)benzyl)(ethyl)amino)pyrazolo[1,5-a]pyrimidine-3-carboxylate

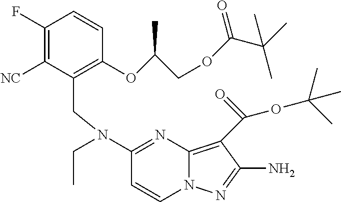 [SMILES: CCN(Cc1c(O[C@@H](C)COC(=O)C(C)(C)C)ccc(F)c1C#N)c1ccn2nc(N)c(C(=O)OC(C)(C)C)c2n1]

At 0° C., to a solution of tert-butyl 2-amino-5-((2-cyano-3-fluoro-6-hydroxybenzyl)(ethyl)amino)pyrazolo[1,5-a]pyrimidine-3-carboxylate (2.1 g, 5.0 mmol), triphenylphosphine (2.0 g, 7.5 mmol) and (R)-propyl 2-hydroxypivalate (1.2 g, 7.5 mmol) in tetrahydrofuran (20 mL), diisopropyl azodicarboxylate (2.0 g, 10.0 mmol) was slowly added; the reaction mixture was raised to room temperature, and reacted with stirring for 6 hours. The reaction progress was monitored by TLC, and the reaction was basically completed. The reaction was cooled to room temperature, concentrated under reduced pressure. The residue was added with water, extracted with ethyl acetate (×3). The ethyl acetate phases were combined, washed with saturated sodium chloride solution (×1), dried over anhydrous sodium sulfate, concentrated under reduced pressure and purified by silica gel column to obtain the title compound (2.3 g, yield 80%).

m/z=569[M+1]⁺.

### Step D: (S)-2-amino-5-((2-cyano-3-fluoro-6-((1-hydroxypropan-2-yl)oxy) benzyl)(ethyl)amino)pyrazolo[1,5-a]pyrimidine-3-carboxylic acid hydrochloride

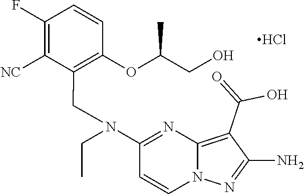 [SMILES: CCN(Cc1c(O[C@@H](C)CO)ccc(F)c1C#N)c1ccn2nc(N)c(C(=O)O)c2n1 ; Cl]

At 0° C., to a solution of (S)-tert-butyl 2-amino-5-((2-cyano-3-fluoro-6-((1-tert-pentanoyloxy)propan-2- yl)oxy)benzyl)(ethyl)amino)pyrazolo[1,5-a] pyrimidine-3-carboxylate (2.0 g, 3.5 mmol) in dichloromethane (20 mL), 4M HCl in 1,4-dioxane (9 mL) was slowly added dropwise. The resulting mixture was stirred at 30° C.for 2 hours to precipitate a large amount of white solid. The progress of the reaction was monitored by TLC and the reaction was complete. The solid was filtered off, washed with MTBE (X₂) and dried under nitrogen in an evacuated filter to obtain the title compound (1.7 g, 100% yield).

m/z=429[M+1]⁺.

### Step E: (S, 1³E, 1⁴E)-1²-amino-2-ethyl-4⁵-fluoro-6-methyl-9-oxo-5,8-dioxa-2-aza-1(5,3)-pyrazolo[1,5-a]pyrimidine-4(1,2)-benzocyclononane-4⁶-carbonitrile

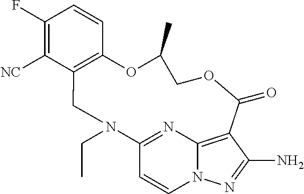 [SMILES: CCN1Cc2c(ccc(F)c2C#N)O[C@@H](C)COC(=O)c2c(N)nn3ccc1nc23]

At room temperature, to a solution of (S)-2-amino-5-((2-cyano-3-fluoro-6-((1-hydroxypropan-2-yl)oxy)benzyl)(ethyl)amino)pyrazolo[1,5-a]pyrimidine-3-carboxylic acid hydrochloride (1.7 g, 3.7 mmol) in tetrahydrofuran (100 mL), 1-ethyl-(3-dimethylaminopropyl)carbodiimide hydrochloride (1.0 g, 5.6 mmol), 4-dimethylaminopyridine (86 mg, 0.7 mmol) and N,N-diisopropylethylamine (2.4 g, 18.5 mmol) were added. The reaction mixture was raised to a temperature of 80° C., and reacted with stirring for 12 hours. The progress of the reaction was monitored by TLC, and the reaction was completed. The reaction was cooled to room temperature, concentrated under reduced pressure. The residue was added with water, extracted with ethyl acetate (×3). The ethyl acetate phases were combined, washed with saturated sodium chloride solution (×1), dried over anhydrous sodium sulfate, concentrated under reduced pressure and purified by silica gel column to obtain the title compound (760 mg, yield 50%).

¹H NMR (400 MHZ, CDCl₃) δ 7.98 (d, J=7.5 Hz, 1H), 7.05-6.88 (m, 2H), 6.30 (d, J=7.5 Hz, 1H), 5.80 (d, J=14.6 Hz, 1H), 4.96 (s, 2H), 4.77 (s, 1H), 4.58 (dd, J=11.5, 3.6 Hz, 1H), 4.25 (dd, J=11.5, 2.8 Hz, 1H), 4.08-4.01 (m, 1H), 3.77 (d, J=14.2 Hz, 2H), 1.59 (d, J=6.3 Hz, 3H), 1.36-1.16 (m, 3H).

m/z=411 [M+1]⁺.

### Example 3 (S, 1³E, 1⁴E, 9Z)-1²-amino-2-ethyl-4⁵-fluoro-9-(hydroxyimino)-6-methyl-5,8-dioxa-2-aza-1(5,3)-pyrazolo[1,5-a]pyrimidine-4(1,2)-benzocyclononane-4⁶-carbonitrile

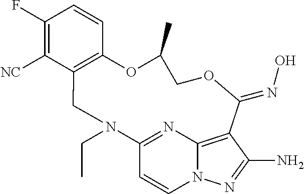 [SMILES: CCN1Cc2c(ccc(F)c2C#N)O[C@@H](C)CO/C(=N\O)c2c(N)nn3ccc1nc23]

At room temperature, the above-mentioned Example 1 (410 mg, 1.0 mmol) was added to dry toluene (15 mL), then phosphorus pentachloride (624 mg, 3.0 mmol) was added. The mixture was heated to 80° C. and stirred for 4 hours, cooled to room temperature. The solvent was spin-dried, and dry acetonitrile (15 mL) was added. Then the mixture was cooled to 0° C., and hydroxylamine hydrochloride (139 mg, 2.0 mmol) and triethylamine (1.0 g, 10.0 mmol) were added, the mixture was rised to room temperature and stirred for 4 hours. The reaction progress was monitored by TLC and the reaction was completed. The reaction was cooled to room temperature, concentrated under reduced pressure. The residue was added with water, extracted with ethyl acetate (×4). The ethyl acetate phases were combined, washed with saturated sodium chloride solution (×1), dried over anhydrous sodium sulfate, concentrated under reduced pressure and purified by silica gel column to obtain the title compound (85 mg, yield 20%).

m/z=426[M+1]⁺.

### Example 4 (S, 1³E, 1⁴E, 9Z)-1²-amino-2-ethyl-4⁵-fluoro-9-(methoxyimino)-6-methyl-5,8-dioxa-2-aza-1(5,3)-pyrazolo[1,5-a]pyrimidine-4(1,2)-benzocyclononane-4⁶-carbonitrile

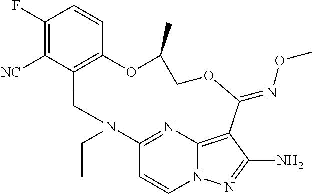 [SMILES: CCN1Cc2c(ccc(F)c2C#N)O[C@@H](C)CO/C(=N\OC)c2c(N)nn3ccc1nc23]

According to the synthesis method of Example 2, hydroxylamine hydrochloride was replaced by O-methylhydroxylamine hydrochloride to obtain (S, 1³E, 1⁴E, 9Z)-1²-amino-2-ethyl-4⁵-fluoro-9-(methoxyimino)-6-methyl-5,8-dioxa-2-aza-1(5,3)-pyrazolo[1,5-a]pyrimidine-4(1,2)-benzcyclononane-4⁶-carbonitrile m/z=440[M+1]⁺.

### Example 5 (S, 1³E, 1⁴E)-1²-amino-2-(ethyl-d₅)-4⁵-fluoro-6-methyl-9-oxo-5,8-dioxa-2-aza-1(5,3)-pyrazolo[1,5-a]pyrimidine-4(1,2)-benzocyclononane-4⁶-carbonitrile

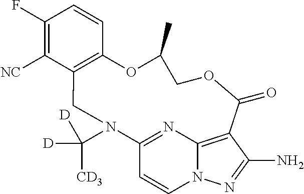 [SMILES: [2H]C([2H])([2H])C([2H])([2H])N1Cc2c(ccc(F)c2C#N)O[C@@H](C)COC(=O)c2c(N)nn3ccc1nc23]

According to the synthesis method of Example 1, 3-bromo-2-((ethylamino)methyl)-4-fluorophenol was replaced by 3-bromo-2-(((ethyl-d₅)amino)methyl)-4-fluorophenol to obtain the title compound.

m/z=416[M+1]⁺.

The following compounds of the Examples were synthesized with reference to the synthesis method of above-mentioned intermediates and Examples:

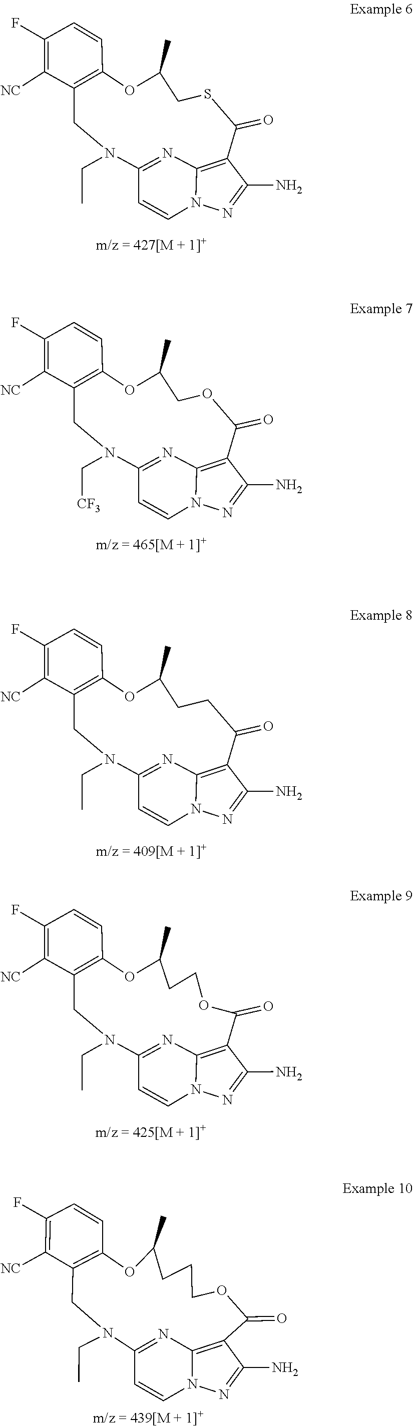 [SMILES: CCN1Cc2c(ccc(F)c2C#N)O[C@@H](C)CSC(=O)c2c(N)nn3ccc1nc23 ; C[C@H]1COC(=O)c2c(N)nn3ccc(nc23)N(CC(F)(F)F)Cc2c(ccc(F)c2C#N)O1 ; CCN1Cc2c(ccc(F)c2C#N)O[C@@H](C)CCC(=O)c2c(N)nn3ccc1nc23 ; CCN1Cc2c(ccc(F)c2C#N)O[C@@H](C)CCOC(=O)c2c(N)nn3ccc1nc23 ; CCN1Cc2c(ccc(F)c2C#N)O[C@@H](C)CCCOC(=O)c2c(N)nn3ccc1nc23]

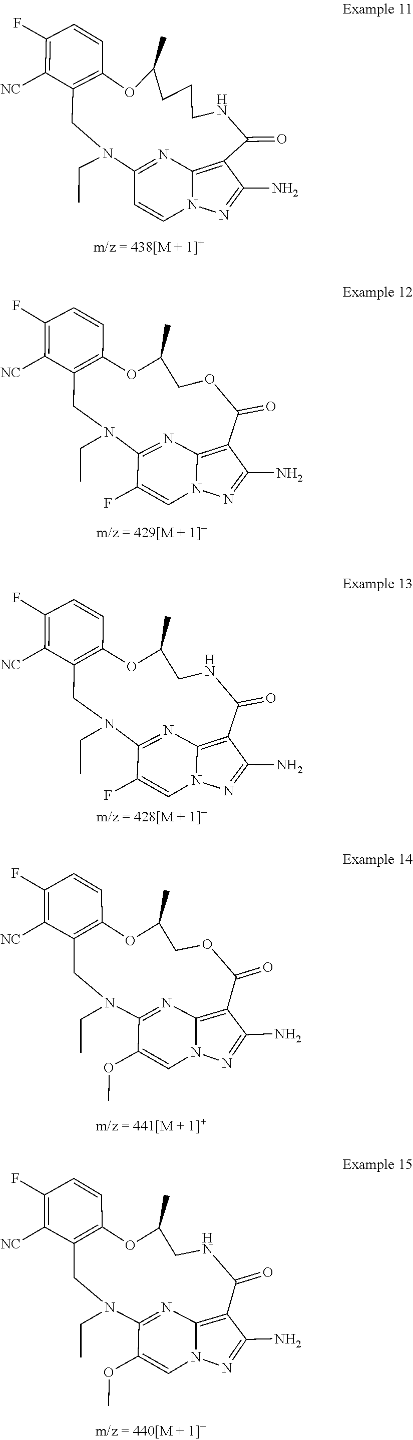 [SMILES: CCN1Cc2c(ccc(F)c2C#N)O[C@@H](C)CCCNC(=O)c2c(N)nn3ccc1nc23 ; CCN1Cc2c(ccc(F)c2C#N)O[C@@H](C)COC(=O)c2c(N)nn3cc(F)c1nc23 ; CCN1Cc2c(ccc(F)c2C#N)O[C@@H](C)CNC(=O)c2c(N)nn3cc(F)c1nc23 ; CCN1Cc2c(ccc(F)c2C#N)O[C@@H](C)COC(=O)c2c(N)nn3cc(OC)c1nc23 ; CCN1Cc2c(ccc(F)c2C#N)O[C@@H](C)CNC(=O)c2c(N)nn3cc(OC)c1nc23]

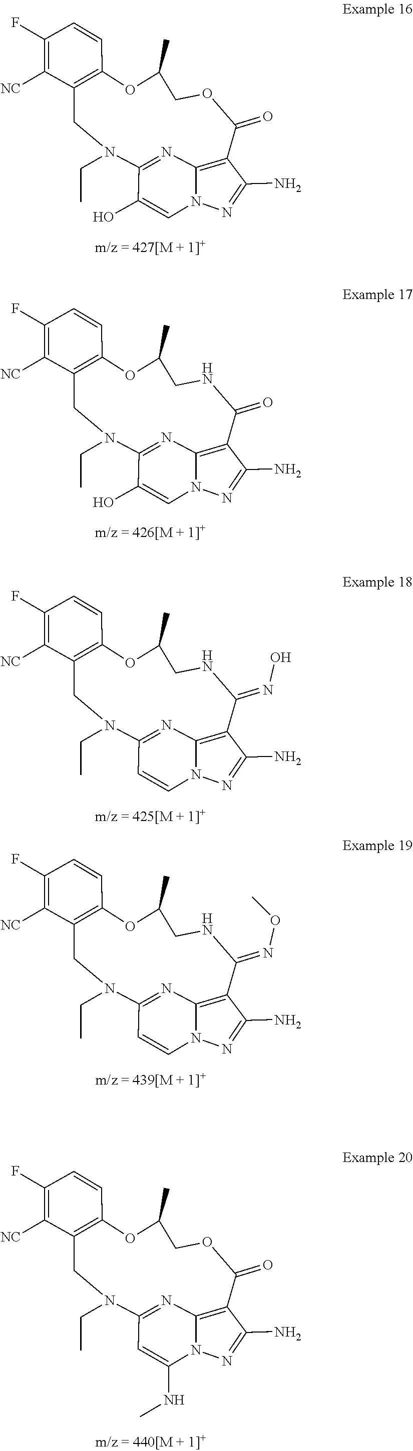 [SMILES: CCN1Cc2c(ccc(F)c2C#N)O[C@@H](C)COC(=O)c2c(N)nn3cc(O)c1nc23 ; CCN1Cc2c(ccc(F)c2C#N)O[C@@H](C)CNC(=O)c2c(N)nn3cc(O)c1nc23 ; CCN1Cc2c(ccc(F)c2C#N)O[C@@H](C)CN/C(=N\O)c2c(N)nn3ccc1nc23 ; CCN1Cc2c(ccc(F)c2C#N)O[C@@H](C)CN/C(=N\OC)c2c(N)nn3ccc1nc23 ; CCN1Cc2c(ccc(F)c2C#N)O[C@@H](C)COC(=O)c2c(N)nn3c(NC)cc1nc23]

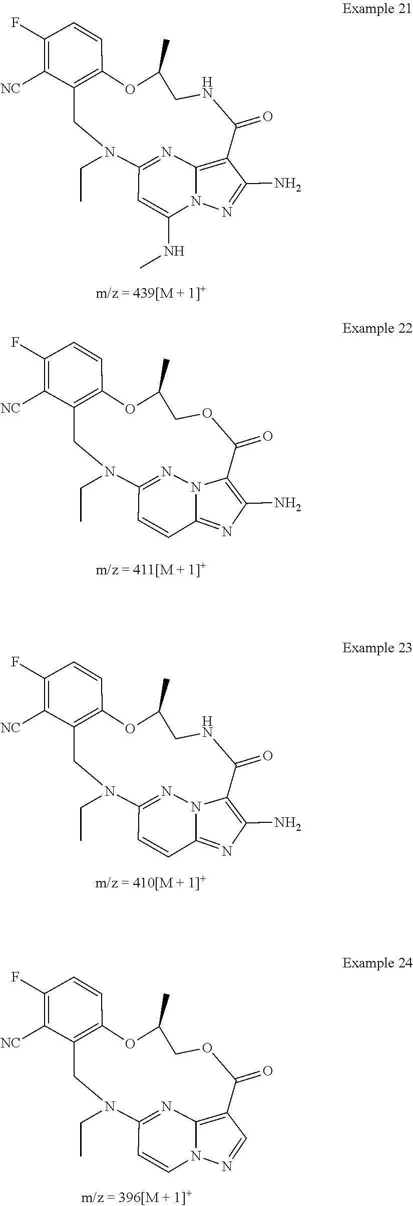 [SMILES: CCN1Cc2c(ccc(F)c2C#N)O[C@@H](C)CNC(=O)c2c(N)nn3c(NC)cc1nc23 ; CCN1Cc2c(ccc(F)c2C#N)O[C@@H](C)COC(=O)c2c(N)nc3ccc1nn23 ; CCN1Cc2c(ccc(F)c2C#N)O[C@@H](C)CNC(=O)c2c(N)nc3ccc1nn23 ; CCN1Cc2c(ccc(F)c2C#N)O[C@@H](C)COC(=O)c2cnn3ccc1nc23]

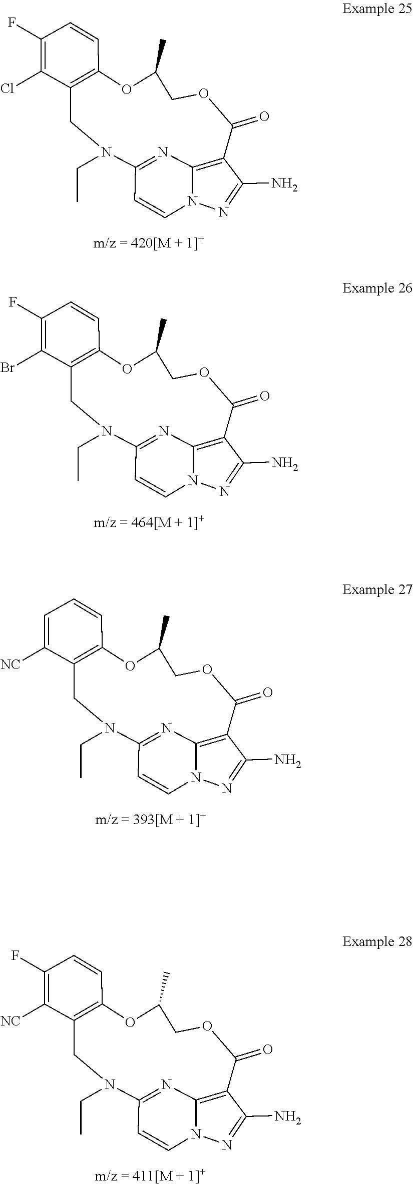 [SMILES: CCN1Cc2c(ccc(F)c2Cl)O[C@@H](C)COC(=O)c2c(N)nn3ccc1nc23 ; CCN1Cc2c(ccc(F)c2Br)O[C@@H](C)COC(=O)c2c(N)nn3ccc1nc23 ; CCN1Cc2c(C#N)cccc2O[C@@H](C)COC(=O)c2c(N)nn3ccc1nc23 ; CCN1Cc2c(ccc(F)c2C#N)O[C@H](C)COC(=O)c2c(N)nn3ccc1nc23]

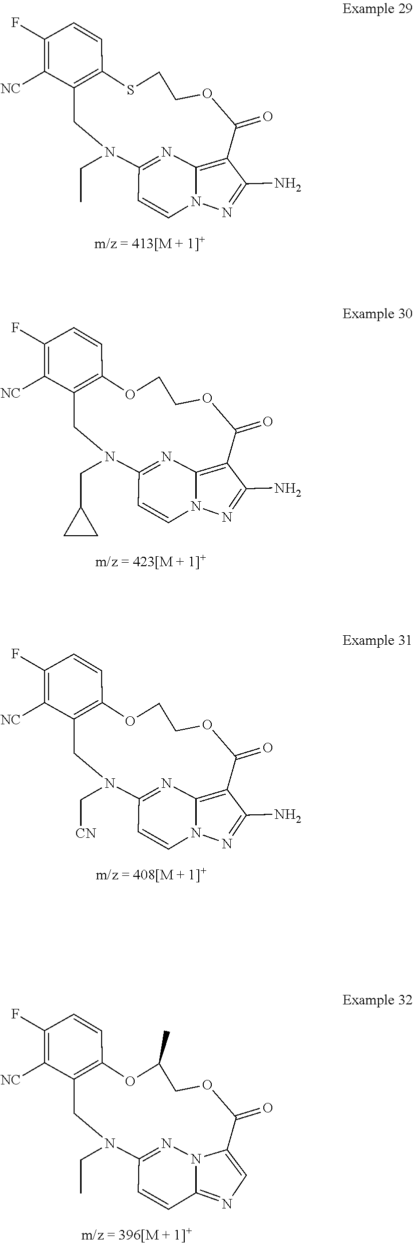 [SMILES: CCN1Cc2c(ccc(F)c2C#N)SCCOC(=O)c2c(N)nn3ccc1nc23 ; N#Cc1c(F)ccc2c1CN(CC1CC1)c1ccn3nc(N)c(c3n1)C(=O)OCCO2 ; N#CCN1Cc2c(ccc(F)c2C#N)OCCOC(=O)c2c(N)nn3ccc1nc23 ; CCN1Cc2c(ccc(F)c2C#N)O[C@@H](C)COC(=O)c2cnc3ccc1nn23]

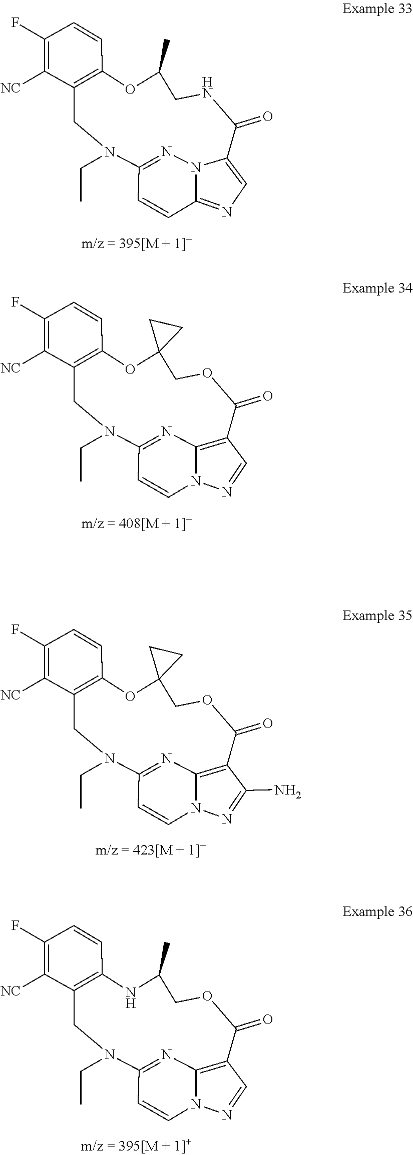 [SMILES: CCN1Cc2c(ccc(F)c2C#N)O[C@@H](C)CNC(=O)c2cnc3ccc1nn23 ; CCN1Cc2c(ccc(F)c2C#N)OC2(CC2)COC(=O)c2cnn3ccc1nc23 ; CCN1Cc2c(ccc(F)c2C#N)OC2(CC2)COC(=O)c2c(N)nn3ccc1nc23 ; CCN1Cc2c(ccc(F)c2C#N)N[C@@H](C)COC(=O)c2cnn3ccc1nc23]

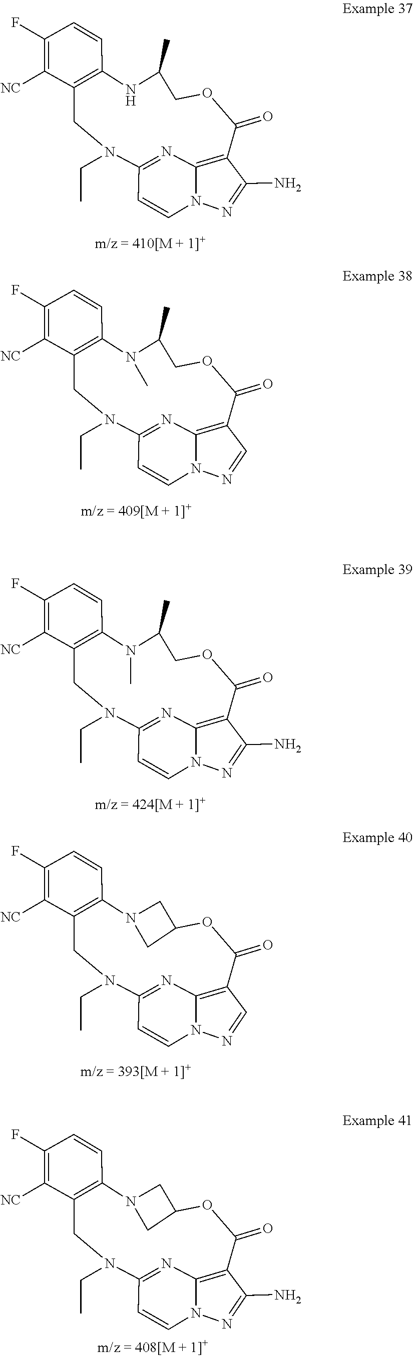 [SMILES: CCN1Cc2c(ccc(F)c2C#N)N[C@@H](C)COC(=O)c2c(N)nn3ccc1nc23 ; CCN1Cc2c(ccc(F)c2C#N)N(C)[C@@H](C)COC(=O)c2cnn3ccc1nc23 ; CCN1Cc2c(ccc(F)c2C#N)N(C)[C@@H](C)COC(=O)c2c(N)nn3ccc1nc23 ; CCN1Cc2c(ccc(F)c2C#N)N2CC(C2)OC(=O)c2cnn3ccc1nc23 ; CCN1Cc2c(ccc(F)c2C#N)N2CC(C2)OC(=O)c2c(N)nn3ccc1nc23]

### EVALUATION OF EFFECTS

### 1. Enzymatic Inhibitory Activity (IC₅₀) Assay of Compounds

Compounds against MET, SRC and CSF1R kinases were screened by use of mobility shift assay. The core of this screening platform was the MSA based on microfluidic chip technology, which applied the basic idea of capillary electrophoresis to a microfluidic environment. The substrate used in the experiment was a polypeptide with fluorescent labeling. Under the catalysis of the enzyme in the reaction system, the substrate was converted into a product, and it carried charge also changed accordingly. MSA utilized the difference in charge between the substrate and the product to separate them, and detect them separately.

The operation method is briefly described as follows:

The powders of the compound were dissolved in 100% DMSO to make a 10 mM stock solution. The initial test concentration of the compound was 10,000 nM, 3-fold serial dilution, 10 concentrations, and repeated hole detection. The serially diluted compounds and kinases were mixed in the Optiplate-384F well plate and incubated at room temperature for 10 minutes, then ATP and Kinase substrate30 (GL Biochem, Cat.117885) mixture were added, mixed homogeneously and then reacted at room temperature for 20 minutes. A stop detection solution was added to stop the enzymatic reaction, and the conversion rate was read with Caliper EZ Reader II.

Data analysis:

$$\% Inhibition = \frac{Conversion \% \_max - Conversion \% \_sample}{Conversion \% \_max - Conversion \% \_min} \times 100$$

wherein: Conversion%_sample is the conversion rate reading of the sample; Conversion%_min: the average value of the negative control wells, representing the conversion rate readings of the wells without enzyme activity; Conversion%_max: the average ratio of the positive control wells, representing the conversion rate readings of the wells without compound inhibition.

Dose-response curve fitting: with the log value of the concentration as the X-axis, and the percentage inhibition rate as the Y-axis, the log(inhibitor) vs. response-Variable slope of the analysis software GraphPad Prism 5 was used to fit the dose-response curve, thereby obtaining IC₅₀ values of each compound against inhibition of enzyme activity.

TABLE 1
c-Met, SRC, and CSFIR Kinase Inhibitory Activities of Compounds

| | c-Met | SRC | CSF1R |
|---|---|---|---|
| Compound | IC₅₀ (nM) | IC₅₀ (nM) | IC₅₀ (nM) |
| TPX-0022ª | 1.8 | 7.5 | 10 |
| Example 1 | 0.93 | 15 | 0.42 |
| Example 2 | 1.8 | 30 | 2.2 |
| Example 5 | N/A | N/A | N/A |
| Example 7 | N/A | N/A | N/A |
| Comparative | 85 | 289 | >10000 |
| Compound 42^{b} | | | |

a: TPX-0022 has the following structure, and its synthesis can refer to Example 5 of WO2019023417A1

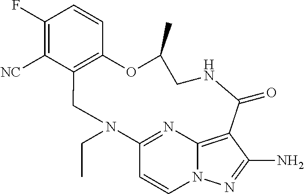 [SMILES: CCN1Cc2c(ccc(F)c2C#N)O[C@@H](C)CNC(=O)c2c(N)nn3ccc1nc23]

b: Comparative Compound 42 has the following structure, and its synthesis refers to Example 1 of WO2019206069A1

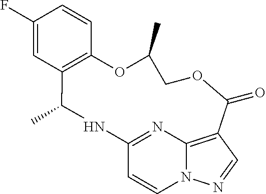 [SMILES: C[C@H]1COC(=O)c2cnn3ccc(nc23)N[C@H](C)c2cc(F)ccc2O1]

### 2. Cell Viability (IC₅₀) Assay of Compounds

### 2.1. Assay of Cell Viability (IC₅₀) Against Human Non-Small Cell Lung Cancer Cells HCC827 Cells

Human non-small cell lung cancer cells, HCC827 cells, were cultured in an incubator (37° C., 5% CO₂) with 1640 medium plus 10% FBS (fetal bovine serum) and 1% P/S (penicillin/streptomycin). In the assay of the compound, the HCC827 cells were plated in a 96-well transparent flat-bottomed black wall plate (Corning, Cat# 3603) at a concentration of 3000 cells/90 uL per well, and the compound was diluted 11 times in a 3-fold gradient starting from 10 mM, and prepared a 10-fold drug solution. Then 10 μL was taken and added to 90 μL of cell culture medium (DMSO final concentration was 0.1%, v/v). After 72 hours of treatment, 100 μL of CellTiter-Glo (Promega, Cat#G7572) was added, cells were lysed by shaking on an orbital shaker for 5 minutes. The cell plate was then left at room temperature for 20 minutes to stabilize the luminescence signal. Luminescence values were read with a SpectraMax Multilabel Microplate Reader (MD, 2104-0010A).

### Data Analysis:

Data were analyzed using GraphPad Prism 5.0 software, and nonlinear S-curve regression was used to fit the data to derive dose-response curves from which IC50 values were calculated.

Cell Viability (%)=(Lum_{drug to be tested}−Lum_{culture medium control})/(Lum_{cell control}−Lum_{culture medium control})×100%.

The test results show that Example 1 has a very obvious inhibitory effect on HCC827 cells.

### 2.2. Assay of Cell Viability (IC₅₀) Against Human Gastric Cancer Cell Line SNU-5 Cells and Ba/F3-TEL-CSF1R

Human gastric cancer cell line SNU-5 culture medium was IMDM basal culture medium supplemented with 10% FBS (fetal bovine serum) and 1% P/S (penicillin/streptomycin), Ba/F3-TEL-CSF1R stably transfected cell line medium was RPMI-1640 medium supplemented with 10% FBS and 1% P/S. Both cell lines were cultured in a carbon dioxide incubator at 37° C. with a CO₂ concentration of 5%.

In the assay of compounds, SNU-5 and Ba/F3-TEL-CSF1R cells were plated in 96-well cell culture plates (Corning, Cat# 3610) at a concentration of 3000 cells/100 μL per well, and cultured overnight. Starting from 200 μM, the compound was subjected to a 3-fold gradient with DMSO, and a total of 9 concentrations were diluted to prepare a 200-fold drug solution. 3 μl of the 200-fold concentration compound was diluted with 197 μl of complete medium to obtain a 3-fold concentration compound. 50 μl of the latter was took and added to the cell well plate, and culture was continued for 72 hours. The cell culture plate was equilibrated to room temperature before assay, and 40 μL of CellTiter-Glo (Promega, Cat# G7571) was added to each well, and the cells were lysed by shaking for two minutes. The cell plate was then left at room temperature for 60 minutes to stabilize the luminescence signal. Luminescence values were read using a PerkinElemer Envision multifunctional plate reader.

Data analysis:
Luminescence values were processed using the following formula:

%Inh=(Max signal−Compound signal)/(Max signal−Min signal)×100
Max signal: value of a DMSO treatment group;
Min signal: value of a blank medium group.

The processed data were analyzed using GraphPad Prism 5.0 software, and nonlinear S-curve regression was used to fit the data to derive dose-response curves from which IC₅₀ values were calculated.

The test results show that the compounds of the Examples have a very obvious inhibitory effect on SNU-5 and Baf3-TEL CSF1R cells; see Table 2 for specific data.

TABLE 2
Inhibitory activity of compounds on cells

| | SNU-5 | Baf3-TEL CSF1R |
|---|---|---|
| Compound | IC₅₀ (nM) | IC₅₀ (nM) |
| TPX-0022 | 6.26 | 12.2 |
| Example 1 | 1.83 | 3.4 |
| Example 2 | 3.36 | 7.0 |

### 3. Synergistic Killing of Tumor Cells with AZD9291

Human lung cancer cells H1975 cells (L858R and T790M double mutations) and HCC827 cells were incubated in an incubator (37° C., 5% CO₂) with 1640 medium plus 10% FBS (fetal bovine serum) and 1% P/S (penicillin/ streptomycin) for culture. In the assay of the compound, H1975 cells or HCC827 were plated in a 96-well plate (Corning) at a concentration of 3000 cells/195 μL per well, and the compound was serially diluted to 11 concentrations starting from 10 mM, and 4 μL of each concentration was added into 96 μL of 1640 cluture medium and diluted to form 25× compound, then 5 μL was took and added to 195 μL of cell culture medium (DMSO final concentration is 0.1%, v/v). After 72 hours of treatment, 35 μL of CellTiter-Blue® (purchased from Promega) was added. The fluorescent signal was measured on Flex Station3 (Molecular Devices) according to the operating procedure of the manual, and the IC₅₀ value of inhibition on cell proliferation of the compound was calculated using GraphPad Prism5.0. The Chou-Talalay combination index method was used to calculate the effect of the combination. The combination index (Col) value 0.9≤CI≤1.1 is an additive effect, 0.8≤CI<0.9 is a low synergistic effect, 0.6≤CI<0.8 is a moderate synergistic effect, and 0.4 <CI<0.6 is highly synergistic, and 0.2≤CI<0.4 is strong synergistic.

The experimental results show that the combination of Example 1 and AZD9291 had a moderate to high synergistic effect on EGFR double mutant cells H1975 (L858R and T790M double mutation) and HCC827 cells (MET overexpression), indicating that the combination of Example 1 and an EGFR inhibitor can overcome the EGFR resistance.

### 4. Pharmacokinetic Experiments

Male SD rats were divided into groups, 3 rats in each group, and the compound of the Examples (1 mg/kg) was given by oral, single gavage and intravenous injection, respectively. The animals were fasted overnight before the experiments, and the fasting time was from 10 hours before administration to 4 hours after administration. Blood was collected at 0.25, 0.5, 1, 2, 4, 8 and 24 hours after administration in the oral group, and at 0.083, 0.25, 0.5, 1, 2, 4, 8 and 24 hours after injection in the intravenous injection group. After anesthesia with isoflurane using a small animal anesthesia machine, 0.3 mL of whole blood was collected through the fundus venous plexus and placed in a heparin anticoagulant tube. The sample was centrifuged at 4° C.and 4000 rpm for 5 minutes, the plasma was transferred to a centrifuge tube, and placed at 80° C. and stored until analysis. Plasma samples were extracted by protein precipitation, and the extract was analyzed by LC/MS/MS.

Example 1 and Example 2 have better pharmacokinetic and pharmacokinetic properties.

### 5. Blood-Brain Distribution Experiments

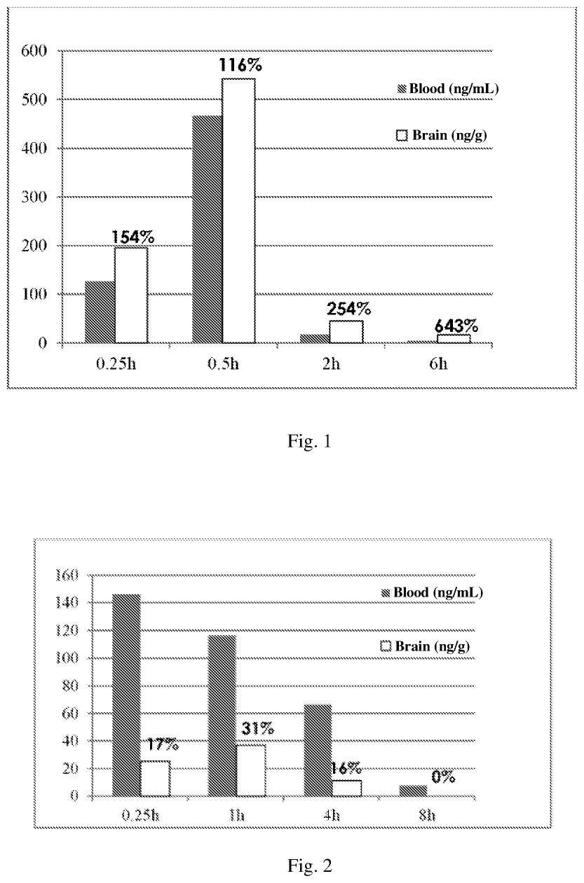

The male SD rats were divided into groups, 12 in each group, and the compound of the Examples (10 mg/kg) was administered orally or by gavage. The animals were fasted overnight before the experiments, and the fasting time was from 10 hours before administration to 4 hours after administration. According to the previous pharmacokinetic data, the rats in the Example 1 group were killed at 0.25, 0.5, 2 and 6 hours after administration; the rats in the TPX-0022 group were killed at 0.25, 1, 4 and 8 hours after administration, and blood and brain tissue were collected. After the samples were processed, they were centrifuged at 4° C., 4000 rpm for 5 min, and the plasma was transferred to a centrifuge tube and stored at −80° C.until analysis. Plasma samples were extracted by protein precipitation, and the extract was analyzed by LC/MS/MS. The results show that the compound of Example 1 can enter the brain tissue, and the drug brain/blood distribution ratio is 1.16-6.4, far exceeding that of TPX-0022. The brain/blood distribution ratios of the compound of Example 1 and TPX-0022 are shown in FIG. 1 and FIG. 2.

Although preferred embodiments of the present disclosure have been disclosed in order to illustrate the present disclosure, those skilled in the art should understand that various modifications, addition and replacement can be made to the present disclosure without departing from the concept and scope of the present disclosure defined in the claims.

1-24. (canceled)

25. A compound represented by formula (I), or a pharmaceutically acceptable salt, solvate, active metabolite, polymorph, isotope marker, isomer, or prodrug thereof, 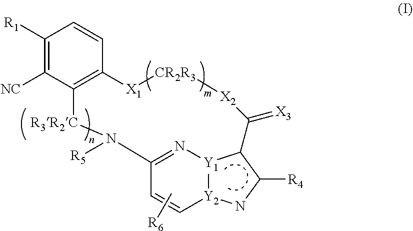
wherein,
X₁ is selected from the group consisting of —O—, —S— and —NR₁₁—;
X₂ is selected from the group consisting of —CH₂—, —O—, —S— and —NH—;
X₃ is selected from the group consisting of O, S and NR₁₀;
Y₁ and Y₂ are different and are selected from the group consisting of C and N; 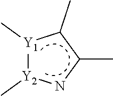
the circular dashed line in indicates that there is a conjugated double bond in the ring;
R₁, R₄, R₅, R₆, R₁₀, and Ru are each independently selected from the following groups which are substituted or unsubstituted: hydrogen, halogen, C_{1˜8} alkyl, deuterated C_{1˜8} alkyl, C_{1˜8} alkoxy, C_{1˜8} alkylamino, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl or cyano;
R₂ and R₃ are each independently selected from the following groups which are substituted or unsubstituted: hydrogen, halogen, C_{1˜8} alkyl, C_{1˜8} alkoxy, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl, cyano; or, together with the C atom and the X₂ group to which they are connected, form a 3˜10 membered cycloalkyl group, a 3˜10 membered heterocyclic group containing at least one heteroatom, or a 5˜10 membered heteroaryl group containing at least one heteroatom;
or, when X₁ is —NR₁₁—, the N atom and the C atom in CR₂R₃ together with R₁₁ and R₁₂ form a 3˜10 membered azacycloalkyl group;
R_{2′} and R_{3′} are each independently selected from the following groups which are substituted or unsubstituted: hydrogen, halogen, C_{1˜8} alkyl, C_{1˜8} alkoxy, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl, cyano; or, together with the connected C and the adjacent N atom, form a 3˜10 membered heterocyclic group containing at least one heteroatom or a 5˜10 membered heteroaryl group containing at least one heteroatom;
m and n represent an integer from 1 to 10; wherein m represents an integer from 3 to 10 when Y₁=C, Y₂=N, X₃=O, X₂=NH and R₆=H;
the substituents of the aforementioned groups may be selected from halogen, C_{1˜8} alkyl, C_{1˜8} haloalkyl, C_{1˜8} alkoxy, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl or cyano.

26. The compound, or a pharmaceutically acceptable salt, solvate, active metabolite, polymorph, isotope marker, isomer, or prodrug thereof according to claim 25, wherein the compound has a structure of the following formula I-1 or I-2: 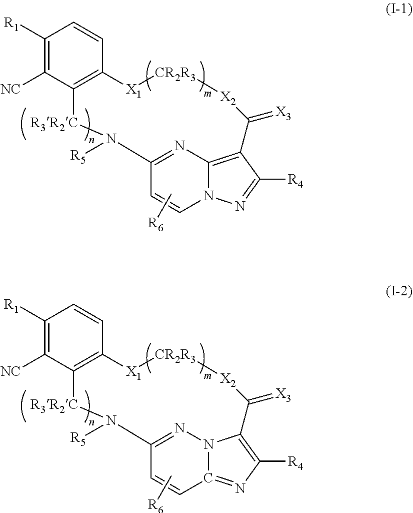
wherein X₁ is selected from the group consisting of —O—, —S— and —NR₁₁—;
X₂ is selected from the group consisting of —CH₂—, —O—, —S— and —NH—;
X₃ is selected from the group consisting of O, S and NR₁₀; 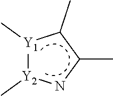
the circular dashed line ir indicates that there is a conjugated double bond in the ring;
R₁, R₄, R₅, R₆, R₁₀, and Ri are each independently selected from the following groups which are substituted or unsubstituted: hydrogen, halogen, C_{1˜8} alkyl, deuterated C_{1˜8} alkyl, C_{1˜8} alkoxy, C_{1˜8} alkylamino, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl or cyano;
R₂ and R₃ are each independently selected from the following groups which are substituted or unsubstituted: hydrogen, halogen, C_{1˜8} alkyl, C_{1˜8} alkoxy, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl, cyano; or, together with the C atom and the X₂ group to which they are connected, form a 3˜10 membered cycloalkyl group, a 3˜10 membered heterocyclic group containing at least one heteroatom, or a 5˜10 membered heteroaryl group containing at least one heteroatom;
or, when X₁ is —NR₁₁—, the N atom and the C atom in CR₂R₃ together with Ril and R₂ form a 3˜10 membered azacycloalkyl group;
R2′ and R₃ are each independently selected from the following groups which are substituted or unsubstituted: hydrogen, halogen, C_{1˜8} alkyl, C_{1˜8} alkoxy, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl, cyano; or, together with the connected C and the adjacent N atom, form a 3˜10 membered heterocyclic group containing at least one heteroatom or a 5˜10 membered heteroaryl group containing at least one heteroatom;
m and n represent an integer from 1 to 10;
the substituents of the aforementioned groups may be selected from halogen, C_{1˜8} alkyl, C_{1˜8} haloalkyl, C_{1˜8} alkoxy, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl or cyano; and
when X₂ is NH, X₃=O, and m=1 to 2, R₆ is not H.

27. The compound, or a pharmaceutically acceptable salt, solvate, active metabolite, polymorph, isotope marker, isomer, or prodrug thereof according to claim 26, wherein the compound has a structure of the following formula I-11;

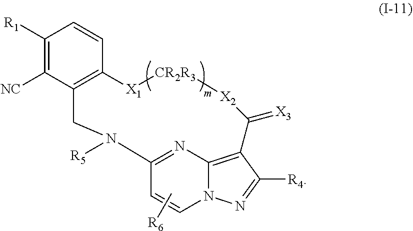

28. The compound, or a pharmaceutically acceptable salt, solvate, active metabolite, polymorph, isotope marker, isomer, or prodrug thereof, according to claim 27, wherein the compound has a structure of the following formula I-12 or I-13 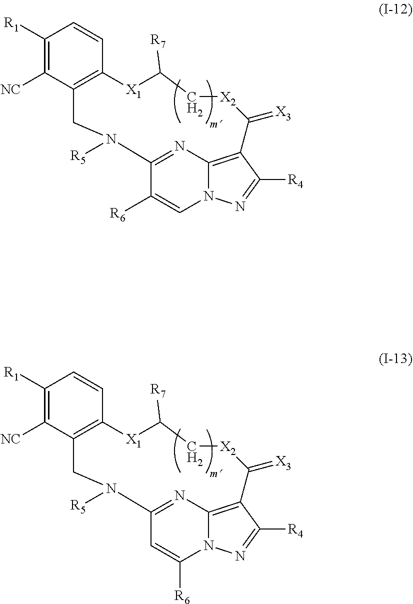
wherein in formulas I-12 and I-13, R₇ is each independently selected from the following groups which are substituted or unsubstituted: hydrogen, halogen, C_{1˜8} alkyl, deuterated C_{1˜8} alkyl, C_{1˜8} alkoxy, C_{1˜8} alkylamino, C_{1˜8} haloalkyl, C_{3˜8} cycloalkyl, C_{3˜8} heterocyclyl, C_{6˜20} aryl, C5˜20 heteroaryl, hydroxyl, mercapto, carboxyl, an ester group, acyl, amino, amido, sulfonyl or cyano; and
m′ represents an integer from 1 to 10.

29. The compound, or a pharmaceutically acceptable salt, solvate, active metabolite, polymorph, isotope marker, isomer, or prodrug thereof according to claim 26, wherein the compound has a structure of the following formula I-21;

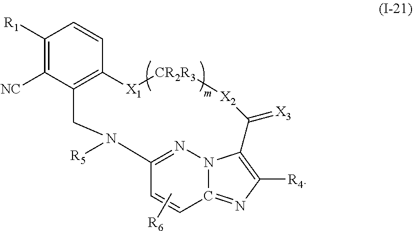

30. The compound, or a pharmaceutically acceptable salt, solvate, active metabolite, polymorph, isotope marker, isomer, or prodrug thereof according to claim 29, wherein the compound has a structure of the formula I-22 or I-23 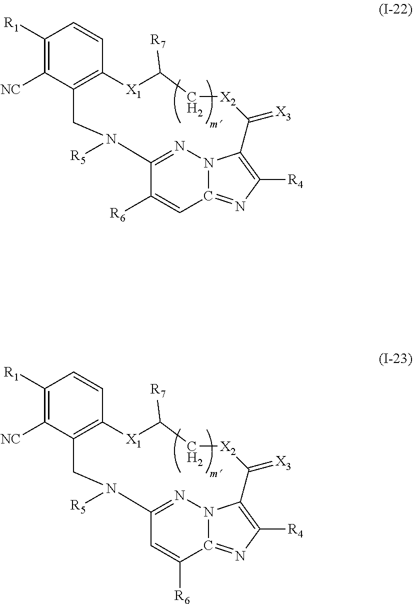
wherein
in formulas I-22 and I-23, R₇ is each independently selected from the following groups which are substituted or unsubstituted: hydrogen, halogen, C_{1˜8} alkyl, deuterated C_{1˜8} alkyl, C_{1˜8} alkoxy, C_{1˜8} alkylamino, C_{1˜8} haloalkyl, C_{3˜8} cycloalkyl, C_{3˜8} heterocyclyl, C_{6˜20} aryl, C_{5˜20} heteroaryl, hydroxyl, mercapto, carboxyl, an ester group, acyl, amino, amido, sulfonyl or cyano;
m′ represents an integer from 1 to 10.

31. The compound, or a pharmaceutically acceptable salt, solvate, active metabolite, polymorph, isotope marker, isomer, or prodrug thereof according to claim 25, wherein R₁ is F.

32. The compound, or a pharmaceutically acceptable salt, solvate, active metabolite, polymorph, isotope marker, isomer, or prodrug thereof according to claim 25, wherein R₅ is selected from the group consisting of C_{1˜8} alkyl, C_{1˜8} haloalkyl, deuterated C_{1˜8} alkyl, C_{3˜8} cycloalkyl C_{1˜8} alkyl or cyano C_{1˜8} alkyl.

33. The compound, or a pharmaceutically acceptable salt, solvate, active metabolite, polymorph, isotope marker, isomer, or prodrug thereof, according to claim 32, wherein R₅ is selected from the group consisting of ethyl, deuteroethyl, cyclopropylmethyl and cyanomethyl.

34. The compound, or a pharmaceutically acceptable salt, solvate, active metabolite, polymorph, isotope marker, isomer, or prodrug thereof according to claim 25, wherein R₄ is hydrogen or amino.

35. The compound, or a pharmaceutically acceptable salt, solvate, active metabolite, polymorph, isotope marker, isomer, or prodrug thereof according to claim 25, wherein R₆ is selected from the group consisting of hydroxyl, amino, C_{1˜8} alkoxy or C_{1˜8} alkylamino.

36. The compound, or a pharmaceutically acceptable salt, solvate, active metabolite, polymorph, isotope marker, isomer, or prodrug thereof according to claim 25, wherein X₁ is —O—.

37. The compound, or a pharmaceutically acceptable salt, solvate, active metabolite, polymorph, isotope marker, isomer, or prodrug thereof according to claim 25, wherein X₂ is —O—;
or, X₂ is —NH—.

38. The compound, or a pharmaceutically acceptable salt, solvate, active metabolite, polymorph, isotope marker, isomer, or prodrug thereof according to claim 25, wherein X₃ is —O—;
or, X₃ is NR₁₀, and Rio is selected from the group consisting of hydroxyl and C_{1˜8} alkoxy.

39. The compound, or a pharmaceutically acceptable salt, solvate, active metabolite, polymorph, isotope marker, isomer, or prodrug thereof according to claim 25, wherein the compound is selected from the group consisting of the following compounds:

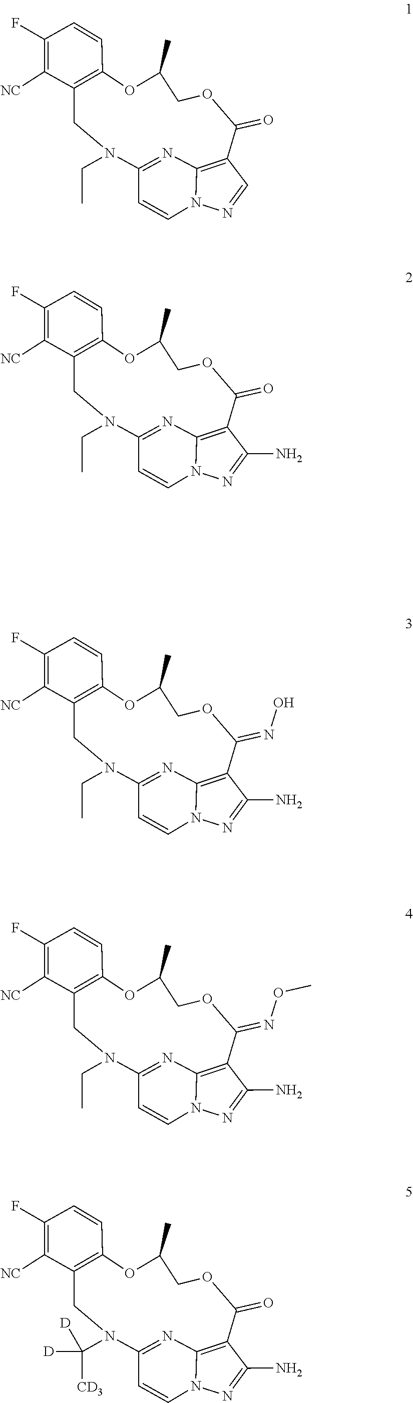 [SMILES: CCN1Cc2c(ccc(F)c2C#N)O[C@@H](C)COC(=O)c2cnn3ccc1nc23 ; CCN1Cc2c(ccc(F)c2C#N)O[C@@H](C)COC(=O)c2c(N)nn3ccc1nc23 ; CCN1Cc2c(ccc(F)c2C#N)O[C@@H](C)CO/C(=N\O)c2c(N)nn3ccc1nc23 ; CCN1Cc2c(ccc(F)c2C#N)O[C@@H](C)CO/C(=N\OC)c2c(N)nn3ccc1nc23 ; [2H]C([2H])([2H])C([2H])([2H])N1Cc2c(ccc(F)c2C#N)O[C@@H](C)COC(=O)c2c(N)nn3ccc1nc23]

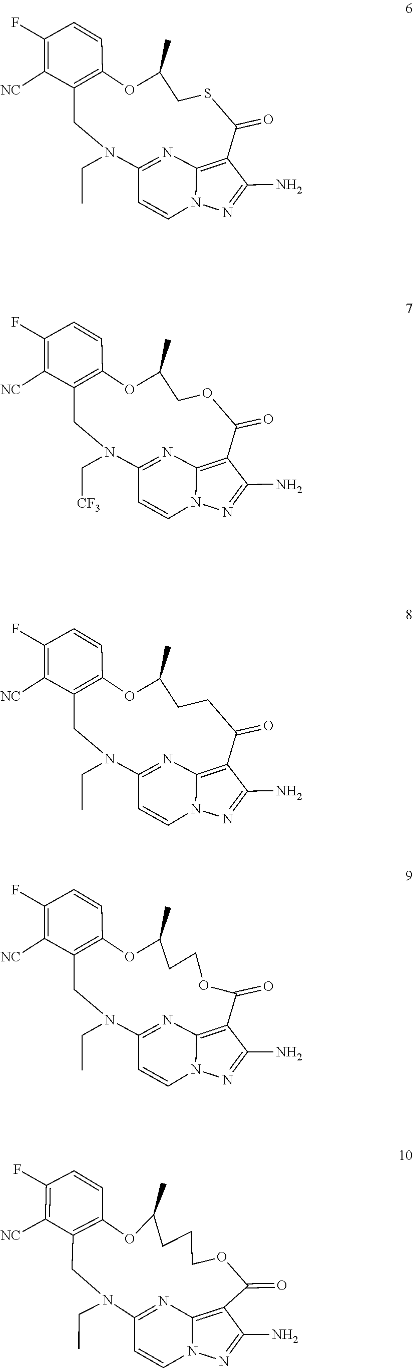 [SMILES: CCN1Cc2c(ccc(F)c2C#N)O[C@@H](C)CSC(=O)c2c(N)nn3ccc1nc23 ; C[C@H]1COC(=O)c2c(N)nn3ccc(nc23)N(CC(F)(F)F)Cc2c(ccc(F)c2C#N)O1 ; CCN1Cc2c(ccc(F)c2C#N)O[C@@H](C)CCC(=O)c2c(N)nn3ccc1nc23 ; CCN1Cc2c(ccc(F)c2C#N)O[C@@H](C)CCOC(=O)c2c(N)nn3ccc1nc23 ; CCN1Cc2c(ccc(F)c2C#N)O[C@@H](C)CCCOC(=O)c2c(N)nn3ccc1nc23]

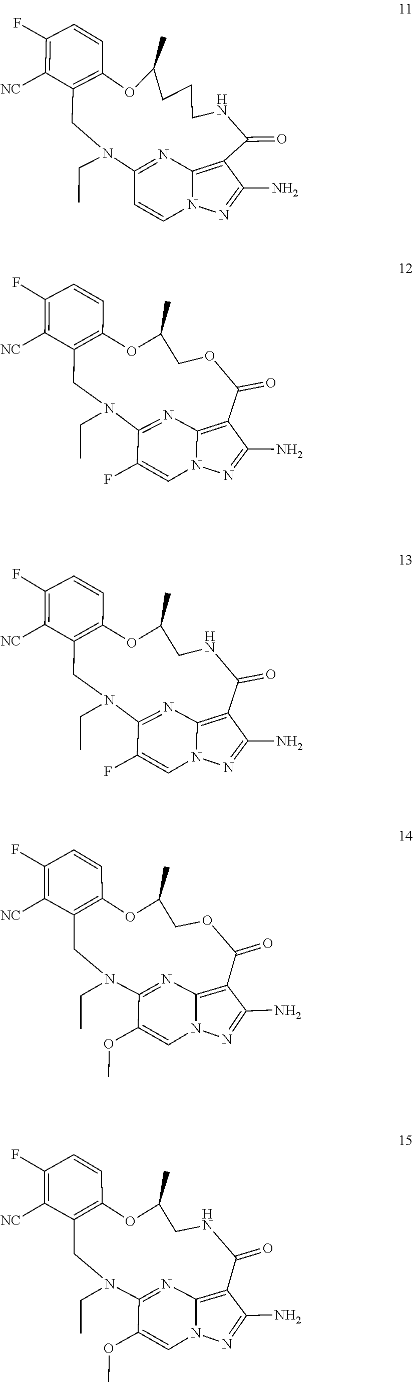 [SMILES: CCN1Cc2c(ccc(F)c2C#N)O[C@@H](C)CCCNC(=O)c2c(N)nn3ccc1nc23 ; CCN1Cc2c(ccc(F)c2C#N)O[C@@H](C)COC(=O)c2c(N)nn3cc(F)c1nc23 ; CCN1Cc2c(ccc(F)c2C#N)O[C@@H](C)CNC(=O)c2c(N)nn3cc(F)c1nc23 ; CCN1Cc2c(ccc(F)c2C#N)O[C@@H](C)COC(=O)c2c(N)nn3cc(OC)c1nc23 ; CCN1Cc2c(ccc(F)c2C#N)O[C@@H](C)CNC(=O)c2c(N)nn3cc(OC)c1nc23]

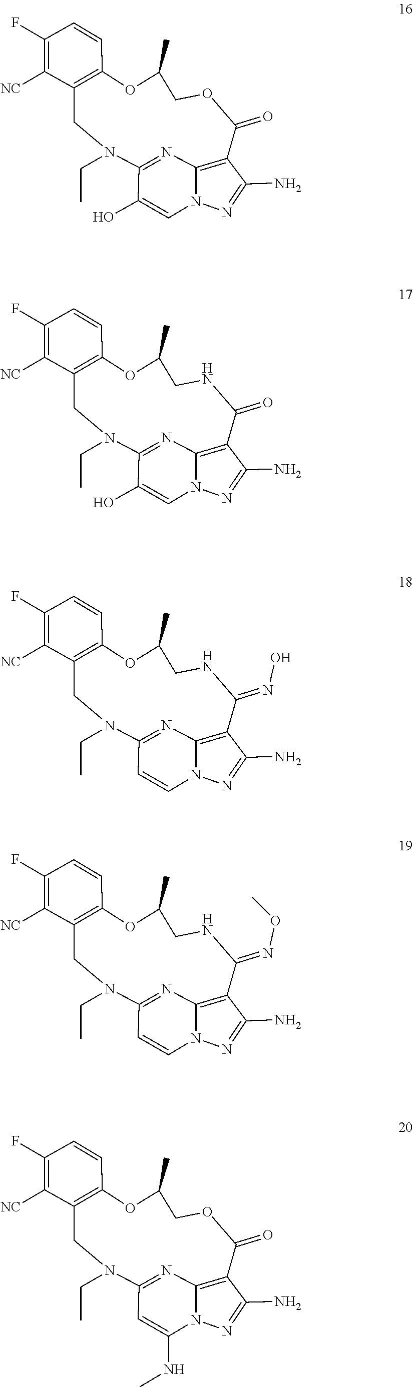 [SMILES: CCN1Cc2c(ccc(F)c2C#N)O[C@@H](C)COC(=O)c2c(N)nn3cc(O)c1nc23 ; CCN1Cc2c(ccc(F)c2C#N)O[C@@H](C)CNC(=O)c2c(N)nn3cc(O)c1nc23 ; CCN1Cc2c(ccc(F)c2C#N)O[C@@H](C)CN/C(=N\O)c2c(N)nn3ccc1nc23 ; CCN1Cc2c(ccc(F)c2C#N)O[C@@H](C)CN/C(=N\OC)c2c(N)nn3ccc1nc23 ; CCN1Cc2c(ccc(F)c2C#N)O[C@@H](C)COC(=O)c2c(N)nn3c(NC)cc1nc23]

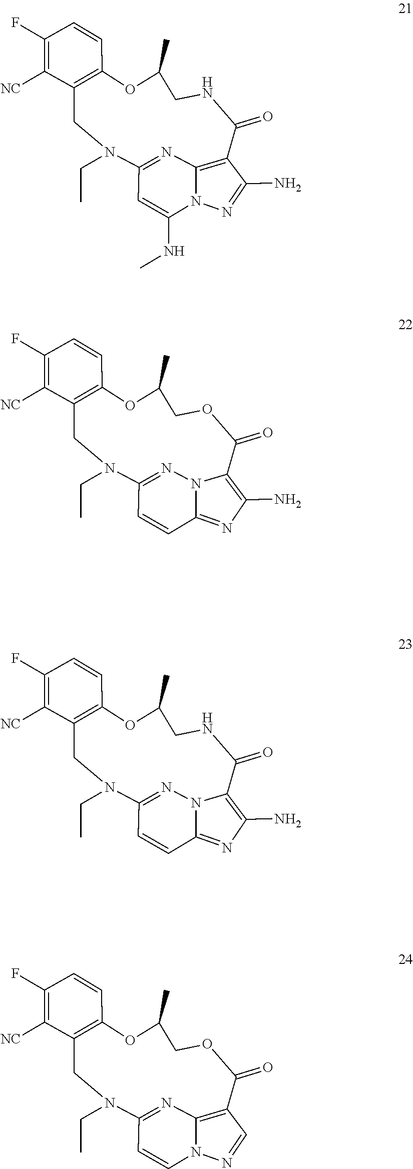 [SMILES: CCN1Cc2c(ccc(F)c2C#N)O[C@@H](C)CNC(=O)c2c(N)nn3c(NC)cc1nc23 ; CCN1Cc2c(ccc(F)c2C#N)O[C@@H](C)COC(=O)c2c(N)nc3ccc1nn23 ; CCN1Cc2c(ccc(F)c2C#N)O[C@@H](C)CNC(=O)c2c(N)nc3ccc1nn23 ; CCN1Cc2c(ccc(F)c2C#N)O[C@@H](C)COC(=O)c2cnn3ccc1nc23]

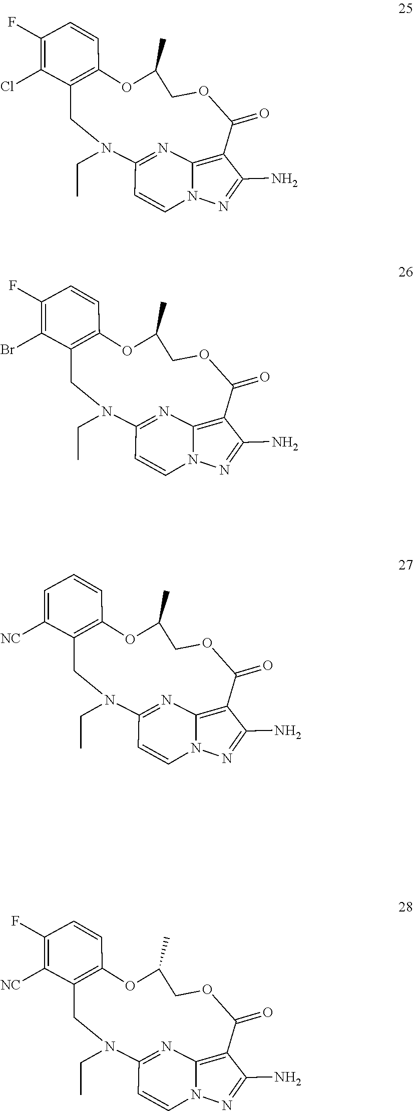 [SMILES: CCN1Cc2c(ccc(F)c2Cl)O[C@@H](C)COC(=O)c2c(N)nn3ccc1nc23 ; CCN1Cc2c(ccc(F)c2Br)O[C@@H](C)COC(=O)c2c(N)nn3ccc1nc23 ; CCN1Cc2c(C#N)cccc2O[C@@H](C)COC(=O)c2c(N)nn3ccc1nc23 ; CCN1Cc2c(ccc(F)c2C#N)O[C@H](C)COC(=O)c2c(N)nn3ccc1nc23]

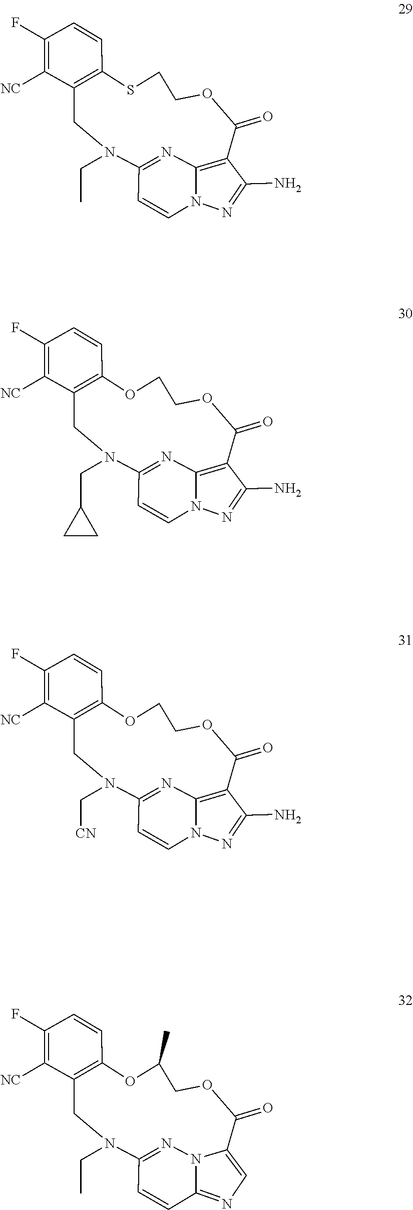 [SMILES: CCN1Cc2c(ccc(F)c2C#N)SCCOC(=O)c2c(N)nn3ccc1nc23 ; N#Cc1c(F)ccc2c1CN(CC1CC1)c1ccn3nc(N)c(c3n1)C(=O)OCCO2 ; N#CCN1Cc2c(ccc(F)c2C#N)OCCOC(=O)c2c(N)nn3ccc1nc23 ; CCN1Cc2c(ccc(F)c2C#N)O[C@@H](C)COC(=O)c2cnc3ccc1nn23]

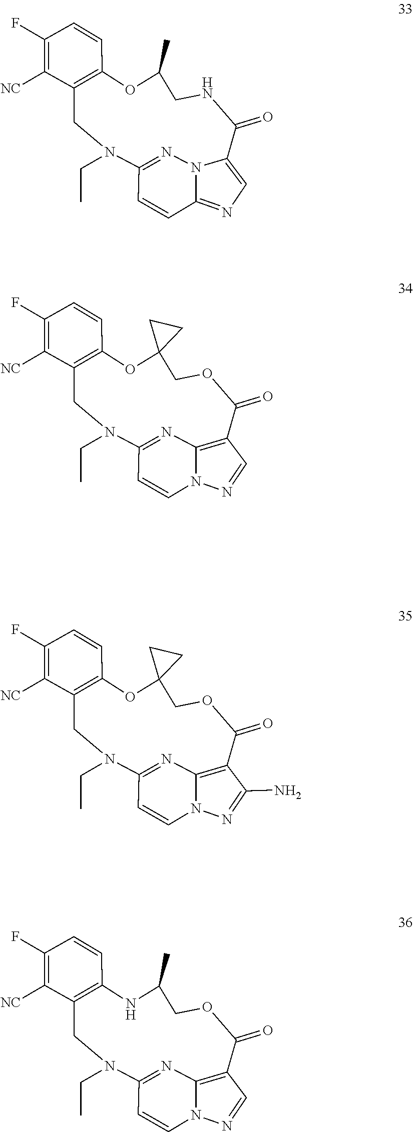 [SMILES: CCN1Cc2c(ccc(F)c2C#N)O[C@@H](C)CNC(=O)c2cnc3ccc1nn23 ; CCN1Cc2c(ccc(F)c2C#N)OC2(CC2)COC(=O)c2cnn3ccc1nc23 ; CCN1Cc2c(ccc(F)c2C#N)OC2(CC2)COC(=O)c2c(N)nn3ccc1nc23 ; CCN1Cc2c(ccc(F)c2C#N)N[C@@H](C)COC(=O)c2cnn3ccc1nc23]

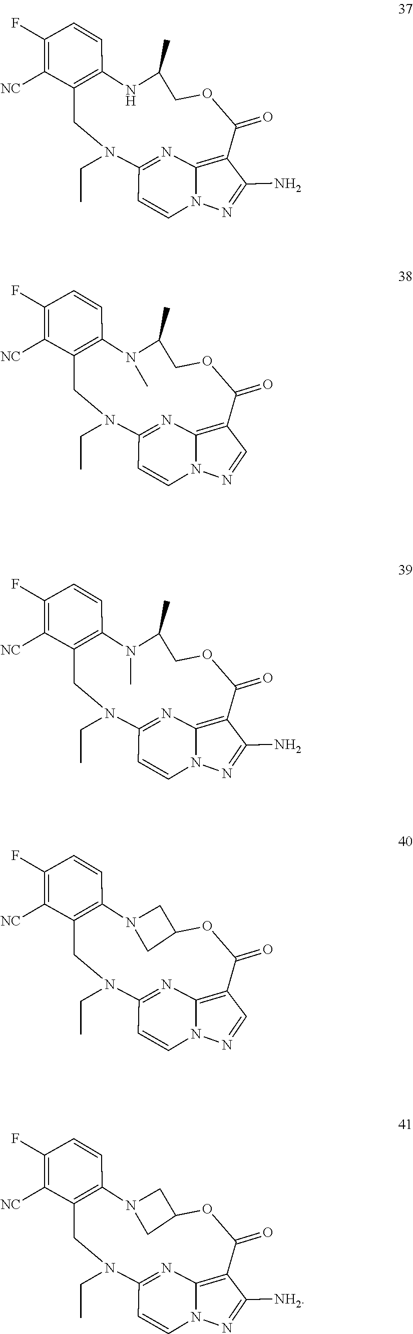 [SMILES: CCN1Cc2c(ccc(F)c2C#N)N[C@@H](C)COC(=O)c2c(N)nn3ccc1nc23 ; CCN1Cc2c(ccc(F)c2C#N)N(C)[C@@H](C)COC(=O)c2cnn3ccc1nc23 ; CCN1Cc2c(ccc(F)c2C#N)N(C)[C@@H](C)COC(=O)c2c(N)nn3ccc1nc23 ; CCN1Cc2c(ccc(F)c2C#N)N2CC(C2)OC(=O)c2cnn3ccc1nc23 ; CCN1Cc2c(ccc(F)c2C#N)N2CC(C2)OC(=O)c2c(N)nn3ccc1nc23]

40. A pharmaceutical composition comprising the compound, or a pharmaceutically acceptable salt, solvate, active metabolite, polymorph, isotope marker, isomer, or prodrug thereof according to claim 25, and a pharmaceutically acceptable carrier.

41. A method for treating tyrosine kinase-mediated diseases, comprising administering to a patient an effective amount of the compound, or a pharmaceutically acceptable salt, solvate, active metabolite, polymorph, isotope marker, isomer, or prodrug thereof according to claim 25.

42. The method according to claim 41, wherein the tyrosine kinase is selected from one or more of the following: SRC, MET, CSF1R, ALK, ROS1, TRKA, TRKB, TRKC, JAK2, SRC, FYN, LYN, YES, FGR, FAK, AXL, ARK5.

43. The method of claim 41, wherein the tyrosine kinase mediated diseases comprise cancer, pain, neurological disorder, autoimmune disease, and inflammation.

44. A combination for treating cancer in a patient, comprising a therapeutically effective amount of a formulation for inhibiting SRC and MET and/or CSF1R and an additional anticancer agent in the same or different specifications, administered simultaneously or separately, wherein the formulation for inhibiting SRC and MET and/or CSF1R comprises the compound, or a pharmaceutically acceptable salt, solvate, active metabolite, polymorph, isotope marker, isomer, or prodrug thereof according to claim 25; and the additional anticancer agent is an EGFR antibody or an EGFR small molecule inhibitor.

45. The combination according to claim 44, wherein the additional anticancer agent is selected from the group consisting of cetuximab, nexituzumab, panitumumab or amivantamab double antibody, afatinib, brigatinib, canertinib, dacomitinib, erlotinib, gefitinib, HKI 357, lapatinib, Osimertinib, Nakotinib, Nazartinib, Neratinib, Omotinib, Pelitinib, PF-06747775, Roxitinib, Vandetanib, Amitinib, Vometinib, Mobocertinib, DZD9008, BEBT-109, lazertinib, CLN-081, WTS-004, JFAN-1001, C-005, XZP-5809-TT1, JRF103, FWD1509, JNJ-372, or a pharmaceutically acceptable salt thereof.
